(19) United States
(12) Patent Application Publication
(10) Pub. No.: US 2026/0355135 A1
(43) Pub. Date: Nov. 12, 2026

(54) COMBINATIONS OF GLYCOLYSIS INHIBITORS/MODULATORS AND PRENYLTRANSFERASE INHIBITORS FOR TREATING BRAIN TUMORS

(71) Applicant: Curatio Therapeutics, LLC, New Haven, CT (US)

(72) Inventors: Carlos Alberto MELO, New Haven, CT (US); Jose CANDAL, New Haven, CT (US); Miguel ORTIZ, New Haven, CT (US); Carlos CANDAL, New Haven, CT (US); Hector CUELLO, New Haven, CT (US); Alfredo CANDAL, New Haven, CT (US); Javier BRUNERO, New Haven, CT (US)

(73) Assignee: Curatio Therapeutics, LLC, New Haven, CT (US)

(21) Appl. No.: 19/688,083

(22) Filed: May 26, 2026

Related U.S. Application Data
(63) Continuation of application No. PCT/US2025/043355, filed on Aug. 25, 2025.
(60) Provisional application No. 63/687,219, filed on Aug. 26, 2024.

(51) Int. Cl. A61K 31/19; A61K 31/045; A61K 31/175; A61K 31/20; A61K 31/4184; A61K 31/495; A61P 35/00; C07K 16/22

(52) U.S. Cl. CPC A61K 31/19; A61K 31/045; A61K 31/175; A61K 31/20; A61K 31/4184; A61K 31/495; A61P 35/00; C07K 16/22

(57) ABSTRACT

The present disclosure refers to therapeutic combinations and pharmaceutical compositions comprising glycolysis inhibitors/modulators and prenyltransferase inhibitors for treating brain tumors. In particular, the disclosure describes therapeutic combinations and pharmaceutical compositions of dichloroacetate (DCA) and perillyl alcohol (POH) useful for treating brain cancers such as glioblastoma multiforme (GBM). Additionally, the disclosure provides methods for treating GBM and other forms of brain cancer using the same.

Continuation of application No. PCT/US2025/043355, filed on Aug. 25, 2025.

### FIELD OF THE INVENTION

The present disclosure relates to therapeutic combinations of glycolysis inhibitors/modulators and prenyltransferase inhibitors and pharmaceutical compositions comprising them for treating brain tumors. The disclosure also provides methods for treating glioblastoma multiforme and other forms of brain cancer using the combinations and pharmaceutical compositions of the disclosure.

### BACKGROUND ART

Glioblastoma, also known as glioblastoma multiforme (GBM) or grade IV astrocytoma, is a fast-growing and aggressive brain tumor. It invades the brain tissue but does not generally spread to distant organs. GBMs can arise in the brain de novo or evolve from lower-grade astrocytoma. In adults, GBM occurs most often in the cerebral hemispheres, especially in the frontal and temporal lobes of the brain. GBM is a devastating brain cancer that can result in death in six months or less, if untreated; hence, it is imperative to seek immediate neuro-oncological and neurosurgical care, as this can impact overall survival.

The mainstay of treatment for GBMs is surgery, followed by radiation and chemotherapy. The primary objective of surgery is to remove as much of the tumor as possible without injuring the surrounding normal brain tissue needed for normal neurological function. However, GBMs are surrounded by a zone of migrating, infiltrating tumor cells that invade surrounding tissues, making it impossible to ever remove the tumor entirely. Surgery provides the ability to reduce the amount of solid tumor tissue within the brain, remove those cells in the center of the tumor that may be resistant to radiation and/or chemotherapy and reduce intracranial pressure. Surgery, by providing a debulking of the tumor, carries the ability to prolong the lives of some patients and improve the quality of remaining life.

In most cases, surgeons perform a craniotomy, opening the skull to reach the tumor site. This is done frequently with computer-assisted image-guidance and at times using intraoperative mapping techniques to determine the locations of the motor, sensory, and speech/language cortex. Intraoperative mapping often involves operating on a patient while they are awake and mapping the anatomy of their language function during the operation. The doctor then decides which portions of the tumor are safe to resect.

After surgery, when the wound is healed, radiation therapy can begin. The goal of radiation therapy is to selectively kill the remaining tumor cells that have infiltrated the surrounding normal brain tissue. In standard external beam radiation therapy, multiple sessions of standard-dose “fractions” of radiation are delivered to the tumor site as well as a margin in order to treat the zone of infiltrating tumor cells. Each treatment induces damage to both healthy and normal tissue.

By the time the next treatment is given, most of the normal cells have repaired the damage, but the tumor tissue has not. This process is repeated for a total of 10 to 60 treatments, usually given once a day, five days a week depending on the type of tumor. The use of radiation therapy provides most patients with improved outcomes and longer survival rates compared to surgery alone or the best supportive care.

Radiosurgery is a treatment method that uses specialized radiation delivery systems to focus radiation at the site of the tumor, while minimizing the radiation dose to the surrounding brain. Radiosurgery may be used in select cases for tumor recurrence, often using additional information derived from MRS or PET scans. It is rarely used in the initial treatment of GBM.

Patients undergoing chemotherapy are administered special drugs designed to kill tumor cells. Chemotherapy with the drug temozolomide is the current standard of treatment for GBM. The drug is generally administered every day during radiation therapy and then for six cycles after radiation during the maintenance phase. Each cycle lasts for 28 days, with temozolomide given the first five days of each cycle, followed by 23 days of rest. Lomustine (chemotherapy) and bevacizumab (targeted therapy) are largely used when the tumor progresses, but exhibit very poor, if any, efficacy.

Thus, GBMs present unique treatment challenges due to localization of tumors in the brain, resistance to chemotherapies, limited capacity of the brain to repair itself, migration of malignant cells to adjacent brain tissue, disrupted tumor blood supply, tumor capillary-related edema and intracranial hypertension, tumor-induced seizures, and neurotoxicity of current therapies. As such, there is a need in the art for additional therapeutic options to treat patients with glioblastoma, which are both efficacious as well as tolerated by patients.

### SUMMARY OF THE INVENTION

In a first aspect, the present invention relates to a co-administered combination comprising a therapeutically effective amount of (a) at least one glycolysis inhibitor/modulator and (b) at least one prenyltransferase inhibitor useful for (i) preventing, inhibiting the progression of or treating brain tumors or (ii) ameliorating the symptoms associated with brain tumors in a subject. In some aspects, the glycolysis inhibitor/modulator is 2-deoxy-D-glucose, dichloroacetate, metformin or a combination thereof. In some aspects, the prenyltransferase inhibitor is 6-gingerol, lonafarnib, perillyl alcohol or a combination thereof. In some aspects, the glycolysis inhibitor/modulator is dichloroacetate and the prenyltransferase inhibitor is perillyl alcohol. In some aspects, the brain tumor is glioblastoma multiforme. In some aspects, the co-administered combination includes or is provided with an additional active agent. In some aspects, the co-administered combination is synergistic. Surprisingly and unexpectedly, the co-administered combination of dichloroacetate and perillyl alcohol is highly effective for treating glioblastoma multiforme when provided at the same time.

In another aspect, the invention refers to a pharmaceutical composition comprising a therapeutically effective amount of (a) at least one glycolysis inhibitor/modulator and (b) at least one prenyltransferase inhibitor, wherein the pharmaceutical composition is useful for (i) preventing, inhibiting the progression of or treating brain tumors or (ii) ameliorating the symptoms associated with brain tumors in a subject. In some aspects, the glycolysis inhibitor/modulator is 2-deoxy-D-glucose, dichloroacetate, metformin or a combination thereof. In some aspects, the prenyltransferase inhibitor is 6-gingerol, lonafarnib, perillyl alcohol or a combination thereof. In some aspects, the glycolysis inhibitor/modulator is dichloroacetate and the prenyltransferase inhibitor is perillyl alcohol. In some aspects, the pharmaceutical composition is synergistic. In some aspects, the brain tumor is glioblastoma multiforme. In some aspects, the pharmaceutical composition is formulated for intranasal administration. In some aspects, the pharmaceutical composition includes or is provided with an additional active agent.

In another aspect, the present invention is directed to a method for (i) preventing, inhibiting the progression of or treating brain tumors or (ii) ameliorating the symptoms associated with brain tumors in a subject in need thereof, which comprises administering to the subject the co-administered combinations or pharmaceutical compositions of the invention.

In another aspect, the present invention refers to a kit including one or more containers comprising a therapeutically effective amount of the co-administered combinations or pharmaceutical compositions of the invention.

The preceding general areas of utility are given by way of example only and are not intended to be limiting on the scope of the present disclosure and appended claims. Additional objects and advantages associated with the compositions, methods, and processes of the present invention will be appreciated by one of ordinary skill in the art considering the instant claims, description, and examples. For example, the various aspects and embodiments of the invention may be utilized in several combinations, all of which are expressly contemplated by the present disclosure. These additional advantages, objects and embodiments are expressly included within the scope of the present invention. The publications and other materials used herein to illuminate the background of the invention, and in particular cases, to provide additional details respecting the practice, are incorporated herein in their entirety by reference.

### BRIEF DESCRIPTION OF THE DRAWINGS

The accompanying drawings, which are incorporated into and form a part of the specification, illustrate several embodiments of the present invention and, together with the description, explain the principles of the invention. The drawings are only for the purpose of illustrating an embodiment of the invention and are not to be construed as limiting the invention. Further objects, features, and advantages of the invention will become apparent from the following detailed description taken in conjunction with the accompanying figures showing illustrative embodiments of the invention, in which:

FIG. 1A. Magnetic Resonance Imaging (MRI) of a patient suffering from glioblastoma. Image of patient's brain immediately following surgical resection (space indicated by white arrow).

FIG. 1B. Magnetic Resonance Imaging (MRI) of a patient suffering from glioblastoma. Image of patient's brain 15 days after surgical resection, with cancer returning at margins of resection (indicated by white arrow).

FIG. 1C. Magnetic Resonance Imaging (MRI) of a patient suffering from glioblastoma. Image of patient's brain 32 days after Image B and 10 days before initiation of treatment with combination of POH and DCA (note that tumor growth was explosive)

FIG. 1D. Magnetic Resonance Imaging (MRI) of a patient suffering from glioblastoma. Image of patient's brain 50 days after initiating treatment (note that tumor growth was unexpectedly halted almost completely). Note: The difference in size of the tumor between Image C and Image D can be attributable to the fact that treatment started 10 days after Image C was taken.

FIG. 2A. MRI image of a patient diagnosed with GBM. Image of patient's brain at moment the treatment started.

FIG. 2B. MRI image of a patient diagnosed with GBM. Image of patient's brain 55 days after starting treatment with a combination of POH and DCA. The results show no progression of the disease (white arrow).

FIG. 3A. Log-phase proliferation of GBM cells with POH treatment. The inhibitory effect of POH on the log-phase growth of different human GBM cells was assessed using a standard high-density cell proliferation assay. LN229 cells were treated for 72 h with a range of POH concentrations (0.1-2 μM) or vehicle control, and proliferation was assessed using the crystal violet staining method.

FIG. 3B. Log-phase proliferation of GBM cells with POH treatment. LN18 cells were treated for 72 h with a range of POH concentrations (0.1-2 μM) or vehicle control, and proliferation was assessed using the crystal violet staining method.

FIG. 3C. Log-phase proliferation of GBM cells with POH treatment. T98G cells were treated for 72 h with a range of POH concentrations (0.1-2 μM) or vehicle control, and proliferation was assessed using the crystal violet staining method. Two independent experiments, n=6-12 per experimental group per independent replicate. Half inhibitory concentrations (IC₅₀) for each cell line were calculated by applying non-linear regressions, based on optimal curve-fitting.

FIG. 4A. Log-phase proliferation of GBM cells with DCA treatment. The inhibitory effect of DCA on the log-phase growth of different human GBM cells was assessed using a standard high-density cell proliferation assay. LN229 cells were treated for 72 h with a range of DCA concentrations (5-100 mM) or vehicle control, and proliferation was assessed using the crystal violet staining method.

FIG. 4B. Log-phase proliferation of GBM cells with DCA treatment. The inhibitory effect of DCA on the log-phase growth of different human GBM cells was assessed using a standard high-density cell proliferation assay. LN18 cells were treated for 72 h with a range of DCA concentrations (5-100 mM) or vehicle control, and proliferation was assessed using the crystal violet staining method.

FIG. 4C. Log-phase proliferation of GBM cells with DCA treatment. The inhibitory effect of DCA on the log-phase growth of different human GBM cells was assessed using a standard high-density cell proliferation assay. T98G cells were treated for 72 h with a range of DCA concentrations (5-100 mM) or vehicle control, and proliferation was assessed using the crystal violet staining method. Two independent experiments, n=6-12 per experimental group per independent replicate. Half inhibitory concentrations (IC₅₀) for each cell line were calculated by applying non-linear regressions, based on optimal curve-fitting.

FIG. 5A. Synergistic effects on log-phase proliferation of GBM cells after a combined POH and DCA treatment. Heat map-based dose-response matrices based on the combination of POH and DCA. Cytostatic activity expressed as percentages of cell growth inhibition (>% inhibitory>grey color intensity).

FIG. 5B. Synergistic effects on log-phase proliferation of GBM cells after a combined POH and DCA treatment. Surface plot for the visualization of synergy scores, representative of the combination of POH with DCA. Data expressed as the average synergy score from the Highest Single Agent (HSA) model. The synergistic effect is evident when the HSA synergy score is greater than 0 (SynergyFinder Software).

FIG. 6A. Representative POH plus DCA combinatorial studies on log-phase proliferation of GBM cells. Visualization of representative drug concentration pairs exemplifying the cooperative effect of POH in addition to selected concentrations of DCA on cell proliferation at 72 h. Specifically, POH 0.5 mM plus DCA 25 mM is depicted.

FIG. 6B. Representative POH plus DCA combinatorial studies on log-phase proliferation of GBM cells. Visualization of representative drug concentration pairs exemplifying the cooperative effect of POH in addition to selected concentrations of DCA on cell proliferation at 72 h. Specifically, POH 0.5 mM plus DCA 50 mM is depicted. Mean with SEM. Two independent experiments, n=6-12 per experimental group per independent replicate. ANOVA (Tukey's multiple comparisons test); ****p<0.0001.

FIG. 7A. Effect of LMP01 on LN229 cell migration. The impact of LMP01 (POH 0.5 mM plus DCA 30 mM) or each monotherapy alone on the migration of LN229 GBM cells was evaluated using a transwell chemotaxis assay. Cells were treated with POH 0.5 mM, DCA 30 mM, LMP01 combined treatment or vehicle control, and their migration was assessed over a 16-h incubation period. Migrating cells were fixed, stained and washed. 4 randomly selected X400-high power fields (HPF) per insert were quantified using QuPath v0.3.0 and normalized to control. Mean with SEM. Two independent experiments, n=12-16 X400-HPF per experimental group per replicate. Kruskal-Wallis test (Dunn's multiple comparisons test); **p<0.01 and ****p<0.0001.

FIG. 7B. Effect of LMP01 on LN229 cell migration. Representative photos from different experimental groups depicting migrated GBM cells.

FIG. 8A. Evaluation of POH on 3D LN229 growth. The effect of POH was assessed on long-term 3D growth of LN229 GBM spheroids. After spheroid generation and randomization, 3D cultures were exposed to POH at different concentrations (0.5, 0.75 or 1 mM) or vehicle control, and their growth was assessed over a period of 14 days, with medium changes (with renewed treatment) occurring twice weekly. 3D GBM spheroid volume over time. Mean with SEM.

FIG. 8B. Evaluation of POH on 3D LN229 growth. The effect of POH was assessed on long-term 3D growth of LN229 GBM spheroids. After spheroid generation and randomization, 3D cultures were exposed to POH at different concentrations (0.5, 0.75 or 1 mM) or vehicle control, and their growth was assessed over a period of 14 days, with medium changes (with renewed treatment) occurring twice weekly. Mean volume analysis between different experimental groups at the end of the experiment (day 14). Two independent experiments, n=4-8 per experiment. Kruskal-Wallis test (Dunn's multiple comparisons test); **p<0.01 or ***p<0.001.

FIG. 9A. Evaluation of DCA on 3D LN229 growth. The effect of DCA was assessed on long-term 3D growth of LN229 GBM spheroids. After spheroid generation and randomization, 3D cultures were exposed to DCA at different concentrations (15, 30 or 60 mM) or vehicle control, and their growth was assessed over a period of 14 days, with medium changes (with renewed treatment) occurring twice weekly. 3D GBM spheroid volume over time. Mean with SEM.

FIG. 9B. Evaluation of DCA on 3D LN229 growth. The effect of DCA was assessed on long-term 3D growth of LN229 GBM spheroids. After spheroid generation and randomization, 3D cultures were exposed to DCA at different concentrations (15, 30 or 60 mM) or vehicle control, and their growth was assessed over a period of 14 days, with medium changes (with renewed treatment) occurring twice weekly. Mean volume analysis between different experimental groups at the end of the experiment (day 14). Two independent experiments, n=4-8 per experiment. Kruskal-Wallis test (Dunn's multiple comparisons test); **p<0.01 or ***p<0.001.

FIG. 10A. Evaluation of LMP01 (POH 0.5 mM and DCA 30 mM) on 3D LN229 growth. The impact of LMP01 (POH 0.5 mM plus DCA 30 mM) or each monotherapy alone was assessed on long-term growth of LN229 GBM spheroids. After spheroid generation and randomization, 3D cultures were exposed to POH 0.5 mM, DCA 30 mM, LMP01 combined treatment or vehicle control, and their growth was assessed over a period of 14 days, with medium changes (with renewed treatment) occurring twice weekly. 3D GBM spheroid volume over time. Mean with SEM.

FIG. 10B. Evaluation of LMP01 (POH 0.5 mM and DCA 30 mM) on 3D LN229 growth. The impact of LMP01 (POH 0.5 mM plus DCA 30 mM) or each monotherapy alone was assessed on long-term growth of LN229 GBM spheroids. After spheroid generation and randomization, 3D cultures were exposed to POH 0.5 mM, DCA 30 mM, LMP01 combined treatment or vehicle control, and their growth was assessed over a period of 14 days, with medium changes (with renewed treatment) occurring twice weekly. Mean volume analysis between different experimental groups at the end of the experiment (day 14). Two independent experiments. n=4-8 per experiment. Kruskal-Wallis test (Dunn's multiple comparisons test); ****p<0.0001.

FIG. 11A. Evaluation of LMP01 (POH 0.5 mM and DCA 30 mM) on 3D LN18 growth. The impact of LMP01 (POH 0.5 mM plus DCA 30 mM) or each monotherapy alone was assessed on long-term growth of LN18 GBM spheroids. After spheroid generation and randomization, 3D cultures were exposed to POH 0.5 mM, DCA 30 mM, LMP01 combined treatment or vehicle control, and their growth was assessed over a period of 10 days, with medium changes (with renewed treatment) occurring twice weekly. 3D GBM spheroid volume over time. Mean with SEM.

FIG. 11B. Evaluation of LMP01 (POH 0.5 mM and DCA 30 mM) on 3D LN18 growth. The impact of LMP01 (POH 0.5 mM plus DCA 30 mM) or each monotherapy alone was assessed on long-term growth of LN18 GBM spheroids. After spheroid generation and randomization, 3D cultures were exposed to POH 0.5 mM, DCA 30 mM, LMP01 combined treatment or vehicle control, and their growth was assessed over a period of 10 days, with medium changes (with renewed treatment) occurring twice weekly. Mean volume analysis between different experimental groups at the end of the experiment (day 10). Two independent experiments, n=4-8 per experiment. Kruskal-Wallis test (Dunn's multiple comparisons test); ****p<0.0001.

FIG. 12A. Evaluation of LMP01 (POH 0.75 mM and DCA 15 mM) on 3D LN229 growth. The impact of LMP01 (POH 0.75 mM plus DCA 15 mM) or each monotherapy alone was assessed on long-term growth of LN229 GBM spheroids. After spheroid generation and randomization, 3D cultures were exposed to POH 0.75 mM, DCA 15 mM, LMP01 combined treatment or vehicle control, and their growth was assessed over a period of 14 days, with medium changes (with renewed treatment) occurring twice weekly. 3D GBM spheroid volume over time. Mean with SEM.

FIG. 12B. Evaluation of LMP01 (POH 0.75 mM and DCA 15 mM) on 3D LN229 growth. The impact of LMP01 (POH 0.75 mM plus DCA 15 mM) or each monotherapy alone was assessed on long-term growth of LN229 GBM spheroids. After spheroid generation and randomization, 3D cultures were exposed to POH 0.75 mM, DCA 15 mM, LMP01 combined treatment or vehicle control, and their growth was assessed over a period of 14 days, with medium changes (with renewed treatment) occurring twice weekly. Mean volume analysis between different experimental groups at the end of the experiment (day 14). Two independent experiments. n=4-8 per experiment. Kruskal-Wallis test (Dunn's multiple comparisons test); ****p<0.0001.

FIG. 13A. Impact of LMP01 (POH 0.75 mM and DCA 15 mM) in comparison to reference TMZ-based chemotherapy on 3D LN229 growth. The effect of LMP01 on the 3D growth of LN229 GBM spheroids was further compared to reference cytotoxic agent TMZ. After spheroid generation and randomization, 3D cultures were exposed to LMP01 (POH 0.75 mM plus DCA 30 mM), TMZ (150 μM) or vehicle control, and their growth was assessed over a period of 14 days, with medium changes (with renewed treatment) occurring twice weekly. 3D GBM spheroid volume over time. Mean with SEM.

FIG. 13B. Impact of LMP01 (POH 0.75 mM and DCA 15 mM) in comparison to reference TMZ-based chemotherapy on 3D LN229 growth. The effect of LMP01 on the 3D growth of LN229 GBM spheroids was further compared to reference cytotoxic agent TMZ. After spheroid generation and randomization, 3D cultures were exposed to LMP01 (POH 0.75 mM plus DCA 30 mM), TMZ (150 μM) or vehicle control, and their growth was assessed over a period of 14 days, with medium changes (with renewed treatment) occurring twice weekly. Mean with SEM. Mean volume analysis between different experimental groups at day 4.

FIG. 13C. Impact of LMP01 (POH 0.75 mM and DCA 15 mM) in comparison to reference TMZ-based chemotherapy on 3D LN229 growth. The effect of LMP01 on the 3D growth of LN229 GBM spheroids was further compared to reference cytotoxic agent TMZ. After spheroid generation and randomization, 3D cultures were exposed to LMP01 (POH 0.75 mM plus DCA 30 mM), TMZ (150 μM) or vehicle control, and their growth was assessed over a period of 14 days, with medium changes (with renewed treatment) occurring twice weekly. Mean with SEM. Mean volume analysis between different experimental groups at day 14. Two independent experiments. n=4-8 per experiment. Kruskal-Wallis test (Dunn's multiple comparisons test); *p<0.0001.

FIG. 14A. Effect of LMP01 in combination with TMZ on LN229 cell migration. The impact of LMP01 in addition to TMZ on the migratory capacity of LN229 GBM cells was evaluated using a transwell chemotaxis assay. Cells were treated with LMP01 (POH 0.5 mM plus DCA 30 mM), TMZ (200 μM), LMP01 plus TMZ or vehicle control, and their migration was assessed over a 16-h incubation period. Migrating cells were fixed, stained and washed. 4 randomly selected X400-high power fields (HPF) per insert were quantified using QuPath v0.3.0 and normalized to control. Two independent experiments, n=12-16 X400-HPF per experimental group per replicate. Kruskal-Wallis test (Dunn's multiple comparisons test); *p<0.05 and ****p<0.0001.

FIG. 14B. Effect of LMP01 in combination with TMZ on LN229 cell migration. Representative photos from different experimental groups depicting migrated GBM cells.

FIG. 15. Histopathological validation of orthotopic LN229 glioblastoma model. (A) Representative H&E-stained coronal section at day 7 post-implantation shows well-demarcated tumor at the striatal injection site, demonstrating characteristic hypercellularity and infiltrative margins. (B) Advanced tumor histopathology from first moribund control animal, featuring: (i) microvascular proliferation, (ii) frequent mitotic figures and, (iii) infiltrative tumor cell clusters. (From left to right: low to high magnification).

FIG. 16. LMP01 significantly prolongs survival in an orthotopic LN229 glioblastoma model. Kaplan-Meier curves show a shift in survival with intranasal (i.n.) LMP01 combination (mean survival: 57 days) and oral+i.n. (mean survival: 54 days) versus vehicle controls (mean survival: 43 days).

FIG. 17. Representative POH plus metformin HCL combinatorial studies on log-phase proliferation of GBM cells. The cooperative effect of a combined administration of POH 0.5 mM plus metformin HCL 50 mM on cell proliferation at 72 h is depicted in comparison with the constituent active agents. ANOVA (Tukey's multiple comparisons test); ****p<0.0001.

FIG. 18. Representative POH plus 2DDG combinatorial studies on log-phase proliferation of GBM cells. The cooperative effect of a combined administration of POH 0.25 mM plus 2DDG 0.5 mM on cell proliferation at 72 h is depicted in comparison with the constituent active agents. ANOVA (Tukey's multiple comparisons test); ****p<0.0001.

FIG. 19. Representative DCA plus lonafarnib combinatorial studies on log-phase proliferation of GBM cells. The cooperative effect of a combined administration of DCA 25 mM plus lonafarnib 10 mM on cell proliferation at 72 h is depicted in comparison with the constituent active agents. ANOVA (Tukey's multiple comparisons test); ****p<0.0001.

FIG. 20. Representative DCA plus 6-gingerol combinatorial studies on log-phase proliferation of GBM cells. The cooperative effect of a combined administration of DCA 50 mM plus 6-gingerol 200 mM on cell proliferation at 72 h is depicted in comparison with the constituent active agents. ANOVA (Tukey's multiple comparisons test); ****p<0.0001.

FIG. 21. Representative 2DDG plus lonafarnib combinatorial studies on log-phase proliferation of GBM cells. The cooperative effect of a combined administration of 2DDG 1.5 mM plus lonafarnib 5 mM on cell proliferation at 72 h is depicted in comparison with the constituent active agents. ANOVA (Tukey's multiple comparisons test); ****p<0.0001.

FIG. 22. Representative 2DDG plus 6-gingerol combinatorial studies on log-phase proliferation of GBM cells. The cooperative effect of a combined administration of 2DDG 1.5 mM plus 6-gingerol 200 mM on cell proliferation at 72 h is depicted in comparison with the constituent active agents. ANOVA (Tukey's multiple comparisons test); ****p<0.0001.

FIG. 23. Representative metformin HCl plus lonafarnib combinatorial studies on log-phase proliferation of GBM cells. The cooperative effect of a combined administration of metformin HCl 50 mM plus lonafarnib10 mM on cell proliferation at 72 h is depicted in comparison with the constituent active agents. ANOVA (Tukey's multiple comparisons test); ****p<0.0001.

FIG. 24. Representative metformin HCl plus 6-gingerol combinatorial studies on log-phase proliferation of GBM cells. The cooperative effect of a combined administration of metformin HCl 50 mM plus 6-gingerol 300 mM on cell proliferation at 72 h is depicted in comparison with the constituent active agents. ANOVA (Tukey's multiple comparisons test); ****p<0.0001.

### DETAILED DESCRIPTION OF THE INVENTION

The following detailed description is provided to assist a person having ordinary skill in the art in practicing the present invention. The skilled person could make modifications and variations to the embodiments described herein without departing from the scope of the present disclosure. All publications, patent applications, patents, figures, and other references mentioned herein are expressly incorporated herein in their entirety by reference.

Presently described are compositions and methods useful in the treatment of several types of brain tumors. In particular, co-administered combinations and pharmaceutical compositions comprising a therapeutically effective amount of (a) at least one glycolysis inhibitor/modulator and (b) at least one prenyltransferase inhibitor, as described herein, are useful for (i) preventing, inhibiting the progression of or treating brain tumors or (ii) ameliorating the symptoms associated with brain tumors. More particularly, dichloroacetate and perillyl alcohol are employed as active agents of the co-administered combinations and pharmaceutical compositions of the invention. The combinations, compositions, and methods of the invention are surprisingly and unexpectedly useful for treating glioblastoma multiforme.

As described below, and as used herein, unless the context indicates otherwise, “co-administration” indicates that the two or more agents, as described herein, are present (e.g., present and/or maintained at therapeutically effective amounts) in the system of the subject at the same time. A person skilled in the art would appreciate that two or more active agents can be administered at substantially the same time or at disparate times, and that the timing of the additional administrations of each of the co-administered active agents will depend upon the particular agents employed and their dosages. For instance, as shown in the Examples below, the co-administered combinations were administered at separate times and different amounts. This is merely an exemplary administration schedule, which would be modified based on the subject, dosages, and the exact agents administered. Thus, co-administration as used herein, and discussed in greater detail below, is merely intended to mean that the co-administered agents are present in the system of the subject simultaneously, e.g., administered to maintain each of the co-administered agents at therapeutically effective amounts in the system of the subject.

The co-administered combinations and pharmaceutical compositions formulations described herein demonstrate a synergistic effect in that the said combinations and compositions achieve at least one of (a) a greater therapeutic effect (i.e., more efficacious) than the additive therapeutic effect obtained by administration of the constituent ingredients alone, (b) a greater therapeutic effect than achieved by administration of a higher dose of the constituent ingredients alone, (c) a similar or greater therapeutic effect but with a decrease in adverse events or side effects relative to that observed by administration of the constituent ingredients alone (i.e., improved therapeutic window) or (d) a similar or greater therapeutic effect at a smaller dose of one or both of the constituent ingredients or a combination thereof. As such, the described combination therapy can also be referred to as, for example, synergistic combinations, synergistic compositions or combinations having synergistically effective amounts.

Unless otherwise defined, all technical and scientific terms used herein have the same meaning as commonly understood by a person of ordinary skill in the art to which the present invention belongs. The terminology used in the disclosure is for describing particular embodiments only and is not intended to limit the scope of the invention.

Where a range of values is provided, it is understood that each intervening value, to the tenth of the unit of the lower limit unless the context clearly dictates otherwise (e.g., as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges and is also encompassed within the invention, subject to any specifically excluded limit in the stated range. Where the stated range includes one or both limits, ranges excluding both limits are also included within the scope of the invention.

### 1. DEFINITIONS OF GENERAL TERMS AND EXPRESSIONS

The following terms are used to describe the present invention. In instances where a term is not specifically defined herein, that term is given the meaning that a person having ordinary skill in the art would understand it to have considering the context to its use in describing the present invention.

2-deoxy-D-glucose: As used herein, the terms “2-deoxy-D-glucose” and “2DDG” refer to a synthetic glucose analog that acts as a non-metabolizable inhibitor of glycolysis, the primary energy pathway for many cells (C₆H₁₂O₅, CAS [154-17-6]).

6-gingerol: As used herein, the term “6-gingerol” refers to a beta-hydroxy ketone that is 5-hydroxydecan-3-one substituted by a 4-hydroxy-3-methoxyphenyl moiety at position 1. It is believed to inhibit adipogenesis (C₁₇H₂₆O₄, CAS [23513-14-6]).

A: As used herein, the articles “a” and the related article “an” refer to one or to more than one (i.e., to at least one) of the grammatical object of the article unless the context clearly indicates otherwise. By way of example, “an element” means one element or more than one element.

About: As used herein, the term “about” should be understood to mean reasonably close when referring to a value or composition that may be within an acceptable error range for said particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the terms can mean up to an order of magnitude or up to 5-fold of a value.

Additional active agent: As used herein, the terms “additional active agent” or “additionally active agent” refer to a compound which is used in combination with the glycolysis inhibitor/modulators and prenyltransferase inhibitors of the disclosure (“primary agents”) to assist the primary agents in effecting their intended preventive, inhibitory or treatment functions. In some embodiments, the additional active agent for use herein includes compounds which have a pharmacological activity similar to the primary agents. In some embodiments, the additional active agent is an anticancer agent (e.g., anti-glioblastoma agent), anti-excitotoxic/calcium modulator, anti-inflammatory/microglial modulator, mitochondrial protectant, neuroprotective/antioxidant agent, neurotrophic/synaptic support agent, palliative agent, other neuroprotective/metabolic modulating medication or a combination thereof.

And/or: As used herein, the term “and/or” should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

At least one: As used herein the term “at least one”, in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); and so on.

Brain tumors: As used herein, the term “brain tumor” refers to an abnormal growth or mass of cells in or around the brain. Brain tumors are classified as malignant (i.e., cancerous) or benign (i.e., non-cancerous). Brain tumors are further classified as primary brain tumors, if they develop in the brain, and secondary or metastatic brain cancers, if they spread to the brain from other parts of the body. According to the present invention, “brain cancer” includes, but is not limited to, astrocytoma, brain stem glioma, chordoma, cranial base tumor craniopharyngioma, diffuse intrinsic pontine glioma, ependymoma, glioblastoma (e.g., glioblastoma multiforme), glioma, hemangioblastoma, lymphoma, medulloblastoma, meningioma, neuronal and glioneuronal tumors, oligodendroglioma, pineal tumors, pituitary adenoma, rhabdoid tumors, vestibular secondary brain tumors due to metastasis, and schwannoma or a combination thereof.

Chemotherapy: As used herein, the term “chemotherapy” refers to the use of low-molecular-mass agents to destroy rapidly dividing cancer cells, employing a variety of mechanisms including DNA interaction and targeted therapeutic approaches. Chemotherapy may be administered by mouth, injection, infusion or applied on the skin, depending on the type and stage of the cancer being treated. It may be provided singly or jointly with other treatments, such as surgery, conventional drugs, gene therapy, immunotherapy, radiotherapy or a combination thereof.

Co-administered combination: As used herein the term “co-administered combination”, and its related terms “co-administration” and “co-administering” or “combination therapy” refer to both concurrent administration (i.e., administration of two or more therapeutic agents at the same time or substantially the same time) and time varied administration (i.e., administration of one or more therapeutic agents at a time different from that of the administration of an additional therapeutic agent or agents), as long as the therapeutic agents are present in the patient to some extent, preferably at effective amounts, at the same time. In some aspects or embodiments, one or more of the described DCA and POH compounds described herein, are co-administered. In some aspects or embodiments, the DCA and POH are also co-administered in combination with at least one additional active agent, especially an anticancer agent. In some aspects or embodiments, the co-administration of compounds results in synergistic activity and/or therapy, including anticancer activity.

Compound: As used herein the term “compound”, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives (including prodrug forms) thereof where applicable, in context. Within its use in context, the term compound refers generally to a single compound, but also may include other compounds such as stereoisomers, regioisomers and/or optical isomers (including racemic mixtures) as well as specific enantiomers or enantiomerically enriched mixtures of disclosed compounds. The term also refers to prodrug forms of compounds described herein which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented within the context of the compound shown.

Comprising: As used herein the terms “comprising” as well as all transitional phrases such as “including,” “carrying,” “having,” “containing,” “involving,” “holding,” “composed of,” and similar are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Dichloroacetate: As used herein, the terms “dichloroacetate” and “DCA” refer to the monocarboxylic acid (CHCl₂CO₂H, CAS [13425-80-4]) with chemical structure:

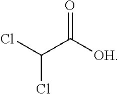

DCA is an analogue of acetic acid, in which 2 of the 3 hydrogen atoms of the methyl group have been replaced by chlorine atoms. The salts and esters of DCA are called dichloroacetates. Salts of DCA have been studied as potential drugs because they inhibit the enzyme pyruvate dehydrogenase kinase by inactivating its kinase activity thus shunting more pyruvate into mitochondria and away from conversion to lactate. Preliminary studies found that DCA can slow the growth of certain tumors in animal studies and in vitro studies. Observations in vitro and of tumors extracted from patients suggest that DCA might act against cancer cells by depolarizing abnormal mitochondria found in glioblastoma cancer cells-allowing the mitochondria to induce the apoptosis of the malignant cells.

Dose: As used herein the term “dose,” refers to the specific amount of a drug administered to a patient at a given time, which can vary based on factors such as body weight, age, and severity of the condition being treated.

In some aspects or embodiments, the term “dose” refers to the amount of (i) DCA, (ii) POH or (iii) a therapeutic combination thereof per kg of the body mass (body weight) administered to a subject over a period of time. A dose can be calculated using the accepted standard weight of a subject. Thus, for example, a dose of POH administered as part of a regimen comprising the administration of a dosage of 3 mg/kg bodyweight to a subject, assuming an ideal body weight of 60 kg, would consist of 180 mg of POH. For example, dosages can be adjusted based on the subject's age, weight, body surface, renal clearance, sex, pathological state, route of administration, concurrent administration of one or more other drugs, and a wide variety of physiologic and psychological factors using methods known in the art (Pan S, et al., Patient Prefer Adherence 2016:10:549-560; Pai M, et al., Pharmacotherapy 2012; 32:856-868, Hacker M, et al., Eds, “Pharmacology: Principles and Practice,” Academic Press, Burlington MA, USA, 2009).

Effective amount: As used herein, the term “effective amount” is used to describe a quantity of a compound, composition or component which, that when used within the context of its intended use, effects an intended result. The term “effective amount” subsumes all other effective concentration terms, which are otherwise described or used in the present application. The term “effective amount” can be used interchangeably with “effective dose,” “therapeutically effective amount” or “therapeutically effective dose.”

Enteral administration: As used herein, the term “enteral administration” and the related term “enterally” refer to the delivery of an active agent via the gastrointestinal tract. Enteral administration includes, but is not limited to, the oral, sublingual, and rectal (e.g., suppository) routes of administration.

Excipient: As used herein, the term “excipient” refers to a substance which supports the absorption of the elements of a pharmaceutical composition, stabilizes said elements, activates or assists in the preparation of the composition. Thus, examples of excipients used in enteral formulations include, but are not limited, to antioxidants (e.g., ascorbic acid, butylated hydroxyanisole), emulsifiers (e.g., soy lecithin, mono- and diglycerides), stabilizers (e.g., modified starches, carrageenan), and thickeners (e.g., mannitol, maltodextrin). Examples of excipients used in parenteral formulations include, but are not limited to, antimicrobial agents (e.g., benzalkonium chloride, metacresol, thimerosal), co-solvents (e.g., ethanol), buffers and pH adjusting factors (e.g., carbonate, citrate, phosphate solutions). Examples of excipients used in topical formulations include, but are not limited to, emollients (e.g., mineral oil, cetyl alcohol, stearic acid) and emulsifiers (e.g., cetearyl alcohol, polysorbate 80, sorbitan stearate).

Farnesyltransferase inhibitor: As used herein, the term “farnesyltransferase inhibitor” and the related term “FTase inhibitor” refer to a class of drugs that induce apoptosis and reduce cell proliferation by blocking the activity of the enzyme farnesyltransferase (FTase). FTase inhibitors prevent the attachment of a farnesyl lipid group to oncogenic Ras proteins. This farnesylation is crucial for Ras proteins to properly function and promote cell growth and survival; by inhibiting it, FTase inhibitors may inhibit tumor growth. FTase inhibitors are classified as direct or indirect. Direct FTase inhibitors include, but are not limited to, α-hydroxy farnesyl phosphonic acid, AZ-5994, BMS-214662, BMS-214662 analogs, BMS-225975, farnesylthiosalicylic acid (FTS), FTS analogs, FPT inhibitor II, FTI-277 hydrochloride, FTI-276 TFA, FTI-2153, geraniol, 6-gingerol, 8-gingerol, gliotoxin, L-744,832, lonafarnib, lonafarnib analogs, manumycin A, manumycin A analogs, PE-344, perillyl alcohol (POH), R115777, R115777 derivatives, salirasib, SCH-115191, TCN-P, TIP-1, tipifarnib, tipifarnib analogs or a combination thereof. Indirect FTase inhibitors include, but are not limited to, bisphosphonates (e.g., alendronate, ibandronate, pamidronate, risedronate, zoledronic acid), BPH-652, lapaquistat acetate, squalestatin 1 (zaragozic acid A), statins (e.g., atorvastatin, cerivastatin, fluvastatin, lovastatin, mevastatin, pitavastatin, rosuvastatin, simvastatin) or a combination thereof.

Gene therapy: As used herein, the term “gene therapy” refers to a family of techniques useful for treating or curing a disease by correcting defective genes, modifying how genes are expressed or introducing new genetic material to a subject to fight a disease. Gene therapy techniques include, but are not limited to, gene addition, gene editing (e.g., CRISPR-Cas systems), gene silencing or a combination thereof. It may be provided singly or jointly with other treatments, such as surgery, conventional drugs, chemotherapy, immunotherapy, radiotherapy or a combination thereof.

Geranylgeranyltransferase inhibitor: As used herein, the term “geranylgeranyl transferase inhibitor” and the related term “GGTase inhibitor” refer to a class of drugs that could induce apoptosis in cells by inhibiting Ki-Ras phenylation. GGTase inhibitors are classified as direct or indirect. Direct GGTase inhibitors include, but are not limited to, 3-PEHPC, GGTI-298, GGTI-2133, GGTI-P4, L-778123, NSC-697923, P61A6 or a combination thereof. Indirect GGTase inhibitors include, but are not limited to, bisphosphonates (e.g., alendronate, ibandronate, pamidronate, risedronate, zoledronic acid), BPH-652, geranylgeranyl pyrophosphate synthase inhibitors, lapaquistat acetate, squalestatin 1 (zaragozic acid A), statins (e.g., atorvastatin, cerivastatin, fluvastatin, lovastatin, mevastatin, pitavastatin, rosuvastatin, simvastatin) or a combination thereof.

Glycolysis inhibitor/modulator: As used herein, the term “glycolysis inhibitor/modulator” refers to a class of drugs that inhibits or modulates the metabolic pathway converting glucose into pyruvate, which plays a pivotal role in the altered metabolism of cancerous cells. Glycolysis inhibitors/modulators are classified as direct glycolysis inhibitors or as indirect glycolysis inhibitors/modulators. Direct glycolysis inhibitors include, but are not limited to, 2-deoxy-D-glucose, 3-bromopyruvate, 3PO, FX11, galloflavin, iodoacetate, lonidamine, oxamate, PFK-15, SF2312, shikonin, WZB117 or a combination thereof. Indirect glycolysis inhibitors/modulators include, but are not limited to, apigenin, berberine, dichloroacetate (DCA), epigallocatechin-3-gallate (EGCG), malonate, metformin, quercetin, resveratrol, vitamin C or a combination thereof.

Immunotherapy: As used herein, the term “immunotherapy” refers to the use of substances for stimulating or suppressing the immune system to fight infection and diseases such as cancer. Several types of immunotherapies are used to treat cancer. These include, but are not limited to, immune checkpoint inhibitors, T-cell transfer therapy, monoclonal antibodies, treatment vaccines, immune system modulators or a combination thereof. It may be provided singly or jointly with other treatments, such as surgery, conventional drugs, chemotherapy, gene therapy, radiotherapy or a combination thereof.

Lonafarnib: As used herein, the term “lonafarnib” refers to a synthetic tricyclic derivative of carboxamide with antineoplastic properties. Lonafarnib binds to and inhibits FTase, an enzyme involved in the post-translational modification and activation of Ras proteins (C₂₇H₃₁Br₂ClN₄O₂, CAS [193275-84-2]).

Metformin: As used herein, the term “metformin” refers to a biguanide antihyperglycemic agent and first-line pharmacotherapy used in the management of type II diabetes (C₄H₁₁N₅, CAS [657-24-9]).

Or: As used herein, the term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Parenteral administration: As used herein, the term “parenteral administration” and the related term “parenterally” refer to the delivery of an active agent by other routes of administration other than enteral and topical administration, usually by injection. Parenteral administration includes, but is not limited to, the epidural, infusion, intraarterial, intraarticular, intracapsular, intracardiac, intracranial, intradermal, intrahepatic, intramuscular, infraorbital, intraperitoneal, intraspinal, intrasternal, intrasynovial, intrathecal, intravenous, subarachnoid, subcapsular, subcutaneous, subcuticular, and transtracheal routes of administration.

Perillyl alcohol: As used herein, the terms “perillyl alcohol” and “POH” refers to the limonene monoterpenoid (C₁₀H₁₆O, CAS [536-59-4]) with chemical structure:

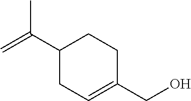

Perillyl alcohol exists as mixture of the R and S racemic enantiomers R-(+)-perillyl alcohol and S-(−)-perillyl alcohol. The active form of POH is S-(−)-perillyl alcohol.

Mammals possess enzymes (P450, liver) to convert limonene to POH. Limonene is formed from geranyl pyrophosphate in the mevalonate pathway. Conversion of limonene to POH is done via hydroxylation by enzymes that belong to the superfamily of cytochrome P450 proteins. POH can be further converted to perillaldehyde (perillyl aldehyde) and perillic acid.

POH has shown antitumor activity in laboratory and animal studies. POH also decreases production of proangiogenic growth factors VEGF and interleukin-8 (IL-8) in vitro. It has been administered nasally for the treatment/prevention of gliomas. For example, POH is being tested currently as a stand-alone therapy in nasal formulations, applied as a 20-minute inhalation, 4 times a day, lifelong.

Pharmaceutically acceptable salt: As used herein, the term “pharmaceutically acceptable salt” refers to a salt form of one or more of the compounds described herein which are presented to, e.g., increase the solubility of the compound in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are commonly employed as neutralization salts. Some common salts contemplated for the compounds described herein include, for example, sodium chloride, sodium bicarbonate, sodium citrate, sodium phosphate, sodium sulfacetamide, sodium valproate, sodium nitroprusside, potassium chloride, potassium citrate, potassium iodide, potassium permanganate, calcium carbonate, calcium gluconate, calcium chloride, calcium lactate, magnesium sulfate, magnesium hydroxide, magnesium citrate, aluminum hydroxide, aluminum chloride, zinc oxide, zinc sulfate, zinc acetate, ferrous sulfate, ferrous fumarate, ferric citrate, iron dextran, diphenhydramine hydrochloride, lidocaine hydrochloride, amitriptyline hydrochloride, metformin hydrochloride, morphine sulfate, codeine sulfate, prednisolone sodium phosphate, codeine phosphate, dexamethasone phosphate, zinc acetate, calcium acetate, sodium acetate, potassium citrate, calcium citrate, sodium lactate, lithium carbonate, calcium carbonate, sodium bicarbonate, ferrous fumarate, dimenhydrinate, chlorpheniramine maleate, hydralazine hydrochloride, bismuth subsalicylate, choline salicylate, magnesium salicylate, lithium citrate, imatinib mesylate, amlodipine besylate, sertraline hydrochloride, loratadine sulfate, and venlafaxine hydrochloride.

Pharmaceutically acceptable vehicle: As used herein, the term “pharmaceutically acceptable vehicle” refers to a substance used in a composition for diluting any of the compounds, excipients or components contained therein to a determined volume or weight. The pharmaceutically acceptable vehicle is an inert substance or a substance with an analogous action to any of the elements comprising the pharmaceutical composition of the present invention. The role of said vehicle is to allow the incorporation of other elements, to allow better dosing and administration or to provide consistency and shape to the composition. Thus, vehicles used in the preparation of enteral formulations include solids (e.g., pills, tablets, osmotic CR capsules), liquids (e.g., solutions, softgels, suspensions, emulsions, syrups, elixirs, tinctures, hydrogels), for oral administration, and ointments, suppositories, enemas, Murphy drips, and nutrient enemas, for rectal administration. Vehicles used in the preparation of parenteral pharmaceutical compositions include aqueous and non-aqueous vehicles (e.g., polyethylene glycol, propylene glycol, ethyl alcohol, peanut oil, corn oil, and soya bean oil). Vehicles used in the preparation of topical formulations include liquids (e.g., lotions, solutions), semisolids (e.g., creams, ointments, gels, pastes), and solids (e.g., powders, patches).

Prenyltransferase: As used herein, the term “prenyltransferase” refers to a family of enzymes that catalyze the posttranslational lipid modification known as protein prenylation. This reaction involves the addition of isoprenoid lipids to substrate proteins, essential for the function of many eukaryotic proteins. Prenyltransferases commonly refer to isoprenyl diphosphate syntheses (IPPSs). IPPSs include farnesyltransferase (FTase), geranylgeranyltransferase type 1 or simply geranylgeranyltransferase (GGTase), and Rab geranylgeranyltransferase (RabGGTase).

Prevention: As used herein, the terms “prevent”, “preventing” and “prevention” refer to inhibiting the inception or decreasing the occurrence or recurrence of a disease (e.g., brain tumors, GBM) in a subject or any symptoms associated with it (e.g., headaches, seizures).

Radiotherapy: As used herein, the term “radiotherapy” refers to the use of high-energy radiation, such as X-rays or protons, to destroy tumor cells while minimizing damage to the surrounding normal tissues. There are two main types: external radiotherapy, delivered by a machine outside the body, and internal radiotherapy (i.e., brachytherapy), where a radioactive source is placed inside the body. It may be provided singly or jointly with other treatments, such as surgery, conventional drugs, chemotherapy, gene therapy, immunotherapy or a combination thereof.

Subject: As used herein the terms “subject” or “patient” are employed to describe an animal, preferably a human or a domesticated animal, to whom treatment, including prophylactic treatment, with the compositions according to the present invention is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present invention, the term patient refers to a human patient unless otherwise stated or implied from the context of the use of the term.

Symptom associated with brain tumor: As used herein, the term “symptom associated with brain tumor” refers to manifestations or indications of a brain tumor perceived and complained about by a subject. These symptoms can develop suddenly or gradually over time. Symptoms associated with brain tumors include, but are not limited to, headaches, seizures, cognitive changes, speech difficulties, vision or sensory changes, muscle weakness and coordination problems, nausea and vomiting, changes in vital signs, fatigue, loss of appetite, vertigo, constipation, anxiety and depression or other clinical manifestations that may affect the quality of life of a subject.

Topical administration: As used herein, the term “topical administration” and the related term “topically” to the delivery of an active agent to a particular place on or in the body, such as the skin or a mucous membrane. Topical administration includes, but is not limited to, the aural, cutaneous, nasal (e.g., intranasal), transdermal, urethral, vaginal, and urethral routes of administration.

Treatment: As used herein, the terms “treat,” “treating” and “treatment” refer to partially or completely alleviating, ameliorating, improving, relieving, delaying the onset of, reducing the severity of, reducing the incidence of one or more symptoms or features of a disease or a combination thereof. For example, “treating” GBM can refer to, e.g., reducing the size of a GBM tumor, increasing the survival, increasing mobility, reducing pain of a subject with GBM or a combination thereof. Thus, in general, the term “treatment” refers to countering the effects caused as a result of the disease or pathological condition of interest in a subject including (i) inhibiting the disease or pathological condition by slowing or stopping the development or progression thereof, (ii) relieving the disease or pathological condition by causing said disease or pathological condition or the symptoms thereof, to regress, (iii) stabilizing the disease or pathological condition, and (iv) a combination thereof.

### 2. COMPOSITIONS

In a first aspect, the present invention relates a co-administered combination comprising a therapeutically effective amount of (a) at least one glycolysis inhibitor/modulator and (b) at least one prenyltransferase inhibitor, wherein the co-administered combination is effective for (i) preventing, inhibiting progression or treating a brain tumor or (ii) ameliorating at least one symptom associated with a brain tumor in a subject. In another aspect, the present invention refers to a pharmaceutical composition comprising a therapeutically effective amount of (a) at least one glycolysis inhibitor/modulator and (b) at least one prenyltransferase inhibitor, wherein pharmaceutical composition is effective for (i) preventing, inhibiting progression or treating a brain tumor or (ii) ameliorating at least one symptom associated with a brain tumor in a subject. The two aspects are illustrated by some embodiments which are common to both aspects. Other embodiments illustrate features that are particular to each of the aspects. For the purpose avoiding unnecessary repetition, the embodiments that are common to the aspects above are discussed together, while the embodiments that are particular to each of aspects are discussed separately. The embodiments are illustrative of present invention and, therefore, do not limit its scope.

### 2.1 In General

In some embodiments, the glycolysis inhibitor/modulator is a direct glycolysis inhibitor or an indirect glycolysis inhibitor/modulator. In some embodiments, the direct glycolysis inhibitor is selected from the group consisting of 2-deoxy-D-glucose, 3-bromopyruvate, 3PO, FX11, galloflavin, lonidamine, oxamate, PFK-15, SF2312, shikonin, and WZB117 or a combination thereof. In some embodiments, the direct glycolysis inhibitor is 2-deoxy-D-glucose. In some embodiments, the indirect glycolysis inhibitor/modulator is selected from the group consisting of apigenin, berberine, DCA, epigallocatechin-3-gallate (EGCG), metformin, quercetin, resveratrol, and vitamin C or a combination thereof. In some embodiments, the indirect glycolysis inhibitor/modulator is DCA, metformin or a combination thereof. In some embodiments, the glycolysis inhibitor/modulator is selected from the group consisting of AZD7545, β-hydroxybutyrate, leelamine (LLM), phenylbutyrate, PS10, and radicicol or a combination thereof.

In some embodiments, the prenyltransferase inhibitor is a direct farnesyltransferase inhibitor or a direct geranylgeranyltransferase inhibitor. In some embodiments, the direct farnesyltransferase inhibitor is selected from the group consisting of BMS-214662, BMS-214662 analogs, 6-gingerol, 8-gingerol, gliotoxin, lonafarnib, lonafarnib analogs, manumycin A, manumycin A analogs, POH, S-(−)-perillyl alcohol, R115777, R115777 derivatives, salirasib, tipifarnib, and tipifarnib analogs or a combination thereof. In some embodiments, the direct farnesyltransferase inhibitor is 6-gingerol, lonafarnib, POH or a combination thereof. In some embodiments, the direct geranylgeranyltransferase inhibitor is selected from the group consisting of 3-PEHPC, GGTI-298, GGTI-2133, GGTI-P4, L-778123 and NSC-697923 or a combination thereof.

In some embodiments, the prenyltransferase inhibitor is an indirect farnesyltransferase inhibitor or an indirect geranylgeranyltransferase inhibitor. In some embodiments, the indirect farnesyltransferase inhibitor or the indirect geranylgeranyltransferase inhibitor is selected from the group consisting of a zoledronic acid and simvastatin or a combination thereof.

In some embodiments, the glycolysis inhibitor/modulator is 2-deoxy-D-glucose, DCA, metformin or a combination thereof and the prenyltransferase inhibitor is 6-gingerol, lonafarnib, POH or a combination thereof. In some embodiments, the glycolysis inhibitor/modulator is DCA and the prenyltransferase inhibitor is POH. In some embodiments, the therapeutically effective amounts of 2-deoxy-D-glucose, DCA, metformin, 6-gingerol, lonafarnib, POH or a combination thereof are synergistic.

In some embodiments, the glycolysis inhibitor/modulator comprises at least one direct glycolysis inhibitor (e.g., 2DDG) and the prenyltransferase inhibitor comprises at least one direct farnesyltransferase inhibitor. In some embodiments, the glycolysis inhibitor/modulator comprises at least one direct glycolysis inhibitor (e.g., 2DDG) and the prenyltransferase inhibitor comprises at least one direct geranylgeranyltransferase inhibitor.

In some embodiments, the glycolysis inhibitor/modulator comprises at least one indirect glycolysis inhibitor/modulator (e.g., DCA, metformin) and the prenyltransferase inhibitor comprises at least one direct farnesyltransferase inhibitor (e.g., lonafarnib, POH, 6-gingerol). In some embodiments, the glycolysis inhibitor/modulator comprises at least one indirect glycolysis inhibitor/modulator (e.g., DCA, metformin) and the prenyltransferase inhibitor comprises at least one direct geranylgeranyltransferase inhibitor.

In some embodiments, the glycolysis inhibitor/modulator comprises at least one direct glycolysis inhibitor (e.g., 2DDG) and the prenyltransferase inhibitor comprises at least one indirect farnesyltransferase inhibitor or an indirect geranylgeranyltransferase inhibitor.

In some embodiments, the glycolysis inhibitor/modulator comprises at least one indirect glycolysis inhibitor/modulator (e.g., DCA, metformin) and the prenyltransferase inhibitor comprises at least one indirect farnesyltransferase inhibitor or an indirect geranylgeranyltransferase inhibitor.

In some embodiments, the glycolysis inhibitor/modulator and the prenyltransferase inhibitor combination comprises at least one (a) DCA plus lonafarnib, (b) DCA plus 6-gingerol, (c) 2DDG plus lonafarnib, (d) 2DDG plus POH, (e) DDG plus 6-gingerol, (f) metformin HCl plus lonafarnib, (g) metformin HCl plus 6-gingerol or a combination thereof. In some embodiments, the glycolysis inhibitor/modulator and the prenyltransferase inhibitor combination is selected from the group consisting of (a) DCA plus lonafarnib, (b) DCA plus 6-gingerol, (c) 2DDG plus lonafarnib, (d) 2DDG plus POH, (e) DDG plus 6-gingerol, (f) metformin HCl plus lonafarnib, and (g) metformin HCl plus 6-gingerol or a combination thereof.

In some embodiments, the brain tumor is brain cancer. In some embodiments, the brain cancer is selected from the group consisting of astrocytoma, brain stem glioma, chordoma, cranial base tumor craniopharyngioma, diffuse intrinsic pontine glioma, ependymoma, glioblastoma, glioblastoma multiforme, glioma, haemangioblastoma, lymphoma, medulloblastoma, meningioma, neuronal and glioneuronal tumors, oligodendroglioma, pineal tumors, pituitary adenoma, rhabdoid tumors vestibular secondary brain tumors due to metastasis and schwannoma or a combination thereof. In some embodiments, brain cancer is glioblastoma multiforme.

In some embodiments, the co-administered combination or the pharmaceutical comprises at least one additional active agent. In some embodiments, the additional active agent comprises an anticancer agent, anti-excitotoxic/calcium modulator, anti-inflammatory/microglial modulator, mitochondrial protectant, neuroprotective/antioxidant agent, neurotrophic/synaptic support agent, other neuroprotective/metabolic modulating medication or a combination thereof.

In some embodiments the anticancer agent comprises everolimus, niacinamide, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEGfilgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, and darbepoetin alfa or a combination thereof.

In some embodiments, the anticancer agent is an anti-glioblastoma agent. In some embodiments, the anti-glioblastoma agent comprises actinidia argot, actinomycin-D, AD-RTC-hIL-12 (Ziopharm), afatinib, antibody-drug conjugates targeting B7-H3, EphA2, GD2, and CSFIR inhibitors, anti-GD2 therapies, aprepitant, ASC40 tablets (Ascletis Pharmaceuticals), asunercept, auranofin, autologous dendritic cell/tumor antigen (ADCTA, SSM Cell Sciences & Technology), AV-GBM-1 (Aivita Biomedical), bevacizumab, buparlisib, cannabinoids (e.g., CBD, THC), capmatinib, captopril, CAR-T therapies, celecoxib, chloroquine, cidofovir, cilengitide, cisplatin combined with etoposide, clofazimine, clomipramine, dabrafenib and trametinib combinations, dapsone, dasatinib, DCVax-L (NorthwesternBio), depatuxizumab mafodotin, digoxin, disulfiram, diltiazem, doxycycline, DSP-7888 dosing emulsion (Sumitomo Pharma), enzastaurin hydrochloride, entrectinib, FGFR inhibitors (e.g., erdafitinib), flubendazole, fluvoxamine, G207 (i.e., an oncolytic herpes simplex virus-1), haloperidol, HDM2 inhibitors, histone methyltransferase inhibitors (e.g., DOTIL), HSP90 inhibitors, hydroxychloroquine, idasanutlin, imipramine, immunocytokines (e.g., IL-2, IL-12 fusions), indopepimut, ipilimumab, itraconazole, lacosamide, LAM561 (Laminar Pharmaceuticals), larotrectinib, lerapolturev, lomustine, lonidamine, marizomib, mebendazole, MEK inhibitors (e.g., cobimetinib, binimetinib, selumetinib, refametinib, TAK-733, ARRY-162, CI-1040), mibefradil and its analogue tetralol derivative NNC-55-0396, minocycline, niraparib, not liposomal transcrocetin (L-TC), nitroxoline, nivolumab, olaparib, omacetaxine mepesuccinate, oncolytic measles virus, oncolytic vesicular stomatitis virus variants, paclitaxel, palbociclib, paliperidone, panobinostat, paxalisib (Kazia Therapeutics), PCV (i.e., procarbazine, CCNU, vincristine), pembrolizumab, plerixafor, plicamycin, proscillaridin A, proteasome inhibitors (e.g., bortezomib), radiolabeled antibodies, regorafenib, reovirus oncolytic virus, repaglinide, ribociclib, ritonavir, sertraline, sirolimus, siroquine, SMARCB1/INII targeting approaches, steroid eye drops, STING agonists (2′3′-cGAMP, c-di-GMP, c-di-AMP, 3′3′-cGAMP, ADU-S100 (MIW815), MK-1454, E7766, SB 11285, GSK3745417, BMS-986301, CF801, IACS-8779, STINGVAX), sunitinib, talazoparib, tasadenoturev, tazemetostat, temozolomide (TMZ), temsirolimus, tepotinib, teserpaturev, thioridazine, tigecycline, tivantinib, tofacitinib, trametinib, trans sodium crocetinate, TVI-Brain-1 (TVAX Biomedical), VBI-1901 (VBI Vaccines), vorasidenib or a combination thereof. In some embodiments, the anti-glioblastoma agent comprises temozolomide, lomustine, bevacizumab or a combination thereof.

In some embodiments, the anti-excitotoxic/calcium modulator comprises agmatine, low dose ketamine, and magnesium (Mg²⁺) or a combination thereof.

In some embodiments, the anti-inflammatory/microglial modulator comprises boswellia serrata (AKBA), curcumin, omega-3 fatty acids (DHA/EPA), quercetin, and resveratrol or a combination thereof.

In some embodiments, the mitochondrial protectant comprises acetyl-L-carnitine, creatine, nicotinamide riboside (NR)/nicotinamide mononucleotide (NMN), and PQQ (pyrroloquinoline quinone) or a combination thereof.

In some embodiments, the neuroprotective/antioxidant agent comprises acetyl-L-carnitine, alpha-lipoic acid, N-acetylcysteine, astaxanthin, clozapine, coenzyme Q10, curcumin, haloperidol, insulin, 1-theanine, magnesium sulfate, melatonin, nicotinamide, omega-3 fatty acids, reduced glutathione, resveratrol, risperidone, selenium, taurine, vitamin E, and vitamin C or a combination thereof. In some embodiments, the neuroprotective/antioxidant agent is selected from the group consisting of acetyl-L-carnitine, alpha-lipoic acid, N-acetylcysteine, curcumin, melatonin, reduced glutathione, resveratrol, vitamin B3, vitamin E or a combination thereof.

In some embodiments, the neurotrophic/synaptic support agent comprises acetyl-L-carnitine, creatine, nicotinamide riboside (NR)/nicotinamide mononucleotide (NMN), and PQQ (pyrroloquinoline quinone) or a combination thereof.

In some embodiments, other the neuroprotective/metabolic modulating medication comprises antidepressants (e.g., amitriptyline, duloxetine, fluoxetine, ketamine, paroxetine, sertraline, venlafaxine), antidiabetic compounds (e.g., pioglitazone, rosiglitazone), antihypertensive compounds (e.g., captopril, carvedilol, enalapril, losartan, nebivolol), antiseizure compounds (e.g., lamotrigine, levetiracetam, topiramate, alproic acid), and N-acetyl-serotonin or a combination thereof. In some embodiments, the neuroprotective/metabolic modulating medication is selected from the group consisting of fluoxetine, losartan, and topiramate or a combination thereof.

In some embodiments, the co-administered combination or the pharmaceutical composition are formulated for enteral, parenteral or topical administration. The appropriate carrier or excipient is selected depending on the desired route of administration.

In some embodiments, POH is formulated at a dose between about 3 to about 20 mg/kg. In some embodiments, DCA is formulated at a dose between about 2 to about 20 mg/kg.

In some embodiments, the subject is human.

### 2.2 Co-Administered Combinations

In some embodiments, the formulations of the glycolysis inhibitor/modulator, the prenyltransferase inhibitor or both in the co-administered combination, further comprise a pharmaceutically acceptable carrier or excipient.

In some embodiments, the formulations of the glycolysis inhibitor/modulator, the prenyltransferase inhibitor or both in the co-administered combination comprise palliative agent. In some embodiments, the palliative agent is selected from the group consisting of analgesics (e.g., acetaminophen, NSAIDs (aspirin, celecoxib, diclofenac, ibuprofen, naproxen), opioids (e.g., codeine, fentanyl, morphine, oxycodone)), corticosteroids (e.g., dexamethasone), levothyroxine, octanoic acid, ondansetron, tramadol, vitamin B3, vitamin D or a combination thereof.

In some embodiments, the formulations of the glycolysis inhibitor/modulator and the prenyltransferase inhibitor are formulated for co-administration via the same or different routes of administration, in the co-administered combination. In some embodiments, the formulation of the glycolysis inhibitor/modulator DCA is formulated for oral or intranasal administration and the formulation of the prenyltransferase inhibitor POH is formulated for intranasal administration, in the co-administered combination.

### 2.3 Pharmaceutical Compositions

In some embodiments, the pharmaceutical composition further comprises a pharmaceutically acceptable carrier or excipient.

In some embodiments, the pharmaceutical composition comprise at least one member selected from the group consisting of codeine, dexamethasone, levothyroxine, octanoic acid, ondansetron, tramadol, vitamin B3, vitamin D or a combination thereof.

In some embodiments, the pharmaceutical composition is formulated for intranasal administration.

Pharmaceutically acceptable carriers that may be used in the formulations of the co-administered combinations and pharmaceutical compositions of the present invention include, but are not limited to, DMSO, soybean oil as a carrier, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Sterile injectable forms in the formulations of the co-administered combinations and pharmaceutical compositions of the present invention include, but are not limited to, aqueous or oleaginous solutions and suspensions. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1, 3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil, castor oil or soybean oil, especially in their polyoxyethylated versions. These oil solutions or suspensions may also contain a long-chain alcohol diluent or dispersant, such as Ph. Helv or similar alcohol.

The formulations of the co-administered combinations and pharmaceutical compositions of the present invention may be orally administered in any orally acceptable dosage form including, but not limited to, capsules, tablets, aqueous suspensions or solutions. In the case of tablets for oral use, carriers which are commonly used include lactose and corn starch.

Lubricating agents, such as magnesium stearate, are also typically added. For oral administration in a capsule form, useful diluents include lactose and dried corn starch. When aqueous suspensions are required for oral use, the active ingredient/agent is combined with emulsifying and suspending agents. If desired, certain sweetening, flavoring or coloring agents may also be added.

Alternatively, the formulations of the co-administered combinations and pharmaceutical compositions of the present invention may be administered in the form of suppositories for rectal administration. These can be prepared by mixing the active agent(s) with a suitable non-irritating excipient, which is solid at room temperature but liquid at rectal temperature and therefore will melt in the rectum to release the drug. Such materials include cocoa butter, beeswax, and polyethylene glycols.

The formulations of the co-administered combinations and pharmaceutical compositions of the present invention may also be administered topically. Suitable topical formulations are readily prepared for each of these areas or organs. Topical application for the lower intestinal tract can be effectuated in a rectal suppository formulation or in a suitable enema formulation. Topically acceptable transdermal patches may also be used.

For topical applications, the formulations of the co-administered combinations and pharmaceutical compositions of the present invention may be formulated in a suitable ointment containing the active component/agent suspended or dissolved in one or more carriers. Carriers for topical administration of the active compounds/agents as described herein include, but are not limited to, mineral oil, liquid petrolatum, DMSO, white petrolatum, propylene glycol, polyoxyethylene, polyoxypropylene compound, emulsifying wax, and water. In some embodiments, the active agents, e.g., active agents in a co-administered combination may be coated onto a stent which is to be surgically implanted into a patient to inhibit or reduce the likelihood of occlusion occurring in the stent in the patient.

Alternatively, the formulations of the co-administered combinations and pharmaceutical compositions of the present invention can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The formulations of the co-administered combinations and pharmaceutical compositions of the present invention may also be administered by nasal aerosol or inhalation. Such compositions or formulations are prepared according to techniques described herein relating to pharmaceutical formulation and may be prepared as solutions in saline, employing benzyl alcohol or other suitable preservatives, absorption promoters to enhance bioavailability, fluorocarbons, and/or other conventional solubilizing or dispersing agents. In some embodiments, the description provides formulations comprising liposomes including an effective amount of the active agent or agents as described herein, including co-administered agents, wherein the liposome formulation is configured or adapted for intranasal delivery or sublingual delivery. In some embodiments, the liposomes further comprise an additional anticancer agent as described herein.

The amount of active agent or agents in the formulations of the co-administered combinations and pharmaceutical compositions of the present invention that may be combined with the carrier materials to produce a single dosage form will vary depending upon the host and disease treated and the particular mode of administration. For example, in the formulations of the co-administered combinations and pharmaceutical compositions should be formulated to contain from between about 0.05 milligram to about 1000 milligrams or more, from between about 1 milligram to about 750 milligrams, and from between about 10 milligrams to about 500 milligrams of active ingredient, alone or in combination with at least one other active compound as described herein.

The active agent or agents in the formulations of the co-administered combinations and pharmaceutical compositions of the present invention may be included in one or more of the active agent categories used in the disclosure. For example, quercetin is described as anti-inflammatory/microglial modulator and as indirect glycolysis modulator/inhibitor, while resveratrol is described as anti-inflammatory/microglial modulator, indirect glycolysis modulator/inhibitor, and neuroprotective agent/antioxidant. Said compound multifunctionality is known in the art and would not affect the scope of the present invention.

The specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject in need of therapy using the formulations of the co-administered combinations and pharmaceutical compositions of the present invention can be treated by administering to the patient (subject) an effective amount of the active agents according to the present invention including their pharmaceutically acceptable salts, solvates or polymorphs, thereof optionally in a pharmaceutically acceptable carrier or diluent, either alone or in combination with other known active agents. These active agents described herein, including co-administered active agents, can be administered by any appropriate route, for example, orally, parenterally, intravenously, intradermally, subcutaneously or topically, including transdermally, in liquid, cream, gel or solid form or by aerosol form.

The active agent or agents, such as the co-administered active agent or agents, are included in the pharmaceutically acceptable carrier or diluent in an amount sufficient to deliver to a patient a therapeutically effective amount for the desired indication, without causing serious toxic effects in the patient treated. In some embodiments, the dose of the active agent or agent(s), e.g. co-administered agent or agents, for all of the herein-mentioned conditions is in the range from between about 10 mg/kg to about 300 mg/kg, between about 0.1 to 250 mg/kg or between about 0.5 to about 200 mg/kg body weight of the recipient/patient per day. A typical topical dosage will range from between 0.01-5% wt/wt in a suitable carrier.

The active agents described herein, e.g. the co-administered active agent or agents, are conveniently administered in any suitable unit dosage form, including but not limited to, one containing less than 1 mg, from between about 1 mg to about 3000 mg or from between about 5 to about 500 mg of active ingredient per unit dosage form.

In some embodiments, the dose of POH of the present disclosure is between 3 mg/kg and about 4 mg/kg, between about 4 mg/kg and about 5 mg/kg, between about 5 mg/kg and about 6 mg/kg, between about 6 mg/kg and about 7 mg/kg, between about 7 mg/kg and about 8 mg/kg, between about 8 mg/kg and about 9 mg/kg, between about 9 mg/kg and about 10 mg/kg, between about 10 mg/kg and about 11 mg/kg, between about 11 mg/kg and about 12 mg/kg, between about 12 mg/kg and about 13 mg/kg, between about 13 mg/kg and about 14 mg/kg, between about 14 mg/kg and about 15 mg/kg, between about 15 mg/kg and about 16 mg/kg, between about 16 mg/kg and about 17 mg/kg, between about 17 mg/kg and about 18 mg/kg, between about 18 mg/kg and about 19 mg/kg or between about 19 mg/kg and about 20 mg/kg.

In some embodiments, the dose of DCA of the present disclosure is between about 2 mg/kg and about 3 mg/kg, between about 3 mg/kg and about 4 mg/kg, between about 4 mg/kg and about 5 mg/kg, between about 5 mg/kg and about 6 mg/kg, between about 6 mg/kg and about 7 mg/kg, between about 7 mg/kg and about 8 mg/kg, between about 8 mg/kg and about 9 mg/kg, between about 9 mg/kg and about 10 mg/kg, between about 10 mg/kg and about 11 mg/kg, between about 11 mg/kg and about 12 mg/kg, between about 12 mg/kg and about 13 mg/kg, between about 13 mg/kg and about 14 mg/kg, between about 14 mg/kg and about 15 mg/kg, between about 15 mg/kg and about 16 mg/kg, between about 16 mg/kg and about 17 mg/kg, between about 17 mg/kg and about 18 mg/kg, between about 18 mg/kg and about 19 mg/kg or between about 19 mg/kg and about 20 mg/kg.

In some embodiments, the dose of POH of the present disclosure is between about 0.10 mg and about 1 mg, between about 1 mg and about 10 mg, between about 10 mg and about 20 mg, between about 20 mg and about 30 mg, between about 30 mg and about 40 mg, between about 40 mg and about 50 mg, between about 50 mg and about 60 mg, between about 60 mg and about 70 mg, between about 70 mg and about 80 mg, between about 80 mg and about 90 mg, between about 90 mg and about 100 mg, between about 100 mg and about 110 mg, between about 110 mg and about 120 mg, between about 120 mg and about 130 mg, between about 130 mg and about 140 mg, between about 140 mg and about 150 mg, between about 150 mg and about 160 mg, between about 160 mg and about 170 mg, between about 170 mg and about 180 mg, between about 180 mg and about 190 mg, between about 190 mg and about 200 mg, between about 200 mg and about 210 mg, between about 210 mg and about 220 mg, between about 220 mg and about 230 mg, between about 230 mg and about 240 mg, between about 240 mg and about 250 mg, between about 250 mg and about 260 mg, between about 260 mg and about 270 mg, between about 270 mg and about 280 mg, between about 280 mg and about 290 mg or between about 290 mg and about 300 mg. In some additional embodiments, the dose of POH of the present disclosure is between about 50 mg and about 51 mg, between about 51 mg and about 52 mg, between about 52 mg and about 53 mg, between about 53 mg and about 54 mg, between about 54 mg and about 55 mg, between about 55 mg and about 56 mg, between about 56 mg and about 57 mg, between about 57 mg and about 58 mg, between about 58 mg and about 59 mg or between about 59 mg and about 60 mg. In some further embodiments, the dose of POH of the present disclosure is about 55 mg.

In some embodiments, the dose of DCA of the present disclosure is between about 300 mg and about 310 mg, between about 310 mg and about 320 mg, between about 320 mg and about 330 mg, between about 330 mg and about 340 mg, between about 340 mg and about 350 mg, between about 350 mg and about 360 mg, between about 360 mg and about 370 mg, between about 370 mg and about 380 mg, between about 380 mg and about 390 mg or between about 390 mg and about 400 mg. In some additional embodiments, the dose of DCA of the present disclosure is between about 330 mg and about 331 mg, between about 331 mg and about 332 mg, between about 332 mg and about 333 mg, between about 333 mg and about 334 mg, between about 334 mg and about 335 mg, between about 335 mg and about 336 mg, between about 336 mg and about 337 mg, between about 337 mg and about 338 mg, between about 338 mg and about 339 mg or between about 339 mg and about 350 mg. In some further embodiments, the dose of DCA of the present disclosure is about 333 mg.

In some embodiments, the dose of POH of the present disclosure is administered at least one, at least two, at least three, at least four, at least five or at least six times per day. In some embodiments, the dose of DCA of the present disclosure is administered at least one, at least two, at least three, at least four, at least five or at least six times per day. In some embodiments, the dose or doses of POH of the present disclosure are administered at least one, at least two, at least three, at least four, at least five, at least six or at least seven times per week. In some embodiments, the dose or doses of DCA of the present disclosure are administered at least one, at least two, at least three, at least four, at least five, at least six or at least seven times per week. In some embodiments, the dose or doses of POH of the present disclosure are administered for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 weeks. In some embodiments, the dose or doses of DCA of the present disclosure are administered for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 weeks. In some embodiments, the dose or doses of POH or DCA of the present disclosure are administered for more than 52 weeks.

In some embodiments, the time lapsing between the administration of the dose of POH and the dose of DCA of the present disclosure is between about 1 minute and about 10 minutes, between about 10 minutes and about 20 minutes, between about 20 minutes and about 30 minutes, between about 30 minutes and about 40 minutes, between about 40 minutes and about 50 minutes, between about 50 minutes and about 60 minutes, between about 60 minutes and about 70 minutes, between about 70 minutes and about 80 minutes, between about 80 minutes and about 90 minutes, between about 90 minutes and about 100 minutes, between about 100 minutes and about 110 minutes, between about 110 minutes and about 120 minutes, between about 120 minutes and about 130 minutes, between about 130 minutes and about 140 minutes, between about 140 minutes and about 150 minutes, between about 150 minutes and about 160 minutes, between about 160 minutes and about 170 minutes, between about 170 minutes and about 180 minutes, between about 180 minutes and about 190 minutes, between about 190 minutes and about 200 minutes, between about 200 minutes and about 210 minutes, between about 210 minutes and about 220 minutes, between about 220 minutes and about 230 minutes or between about 230 minutes and about 240 minutes. In some additional embodiments, the dose of POH and the dose of DCA of the present disclosure are administered simultaneously. In some other embodiments, the dose of POH is administered before the dose of DCA of the present disclosure. In some other embodiments, the dose of POH is administered after the dose of DCA of the present disclosure.

The active ingredient(s)/agent(s) as described herein, including co-administered active ingredients/agents, may be administered to achieve peak plasma concentrations of the active compound of about 0.00001-30 mM, preferably about 0.1-30 μM. This may be achieved, for example, by the intravenous injection of a solution or formulations described herein, including the co-administered formulations, optionally in saline or an aqueous medium, or administered as a bolus of the active ingredient. Oral administration is also appropriate to generate effective plasma concentrations of active agent.

The concentration of active agents in the drug composition or formulation will depend on absorption, distribution, inactivation, and excretion rates of the drug, as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once or may be divided into several smaller doses to be administered at varying intervals of time.

In any aspect or embodiment described herein, the concentration of the POH active agent is broadly from about 10% to 97%, with a preferred concentration such as 95 to 97%. The concentration of the DCA active agent is broadly from about 10% to 99%, with a preferred range such as 95% to 99%.

Oral compositions will generally include an inert diluent or an edible carrier. They may be enclosed in gelatin capsules or compressed into tablets. For oral therapeutic administration, the active agent/ingredient or its prodrug derivative can be incorporated with excipients and used in the form of tablets, troches or capsules. Pharmaceutically compatible binding agents, and/or adjuvant materials can be included as part of the composition.

The tablets, pills, capsules, troches, and similar formulations may contain any of the following ingredients, or compounds of a similar nature: a binder such as microcrystalline cellulose, gum tragacanth or gelatin; an excipient such as starch or lactose, a dispersing agent such as alginic acid, Primo gel or corn starch; a lubricant such as magnesium stearate or Sterotes; a glidant such as colloidal silicon dioxide; a sweetening agent such as sucrose or saccharin; or a flavoring agent such as peppermint, methyl salicylate or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac or enteric agents.

The active agent/ingredients, e.g., the active agent/ingredient in a co-administered formulation or pharmaceutically acceptable salt thereof can be administered as a component of an elixir, suspension, syrup, wafer, chewing gum or similar. A syrup may contain, in addition to the active agent or ingredient, sucrose as a sweetening agent, certain preservatives, dyes and colorings, and flavors.

Solutions or suspensions used for parenteral, intradermal, subcutaneous or topical application can include the following components: a sterile diluent such as water for injection, saline solution, fixed oils (e.g., soybean oil), polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The parenteral preparation can be enclosed in ampoules, disposable syringes or multiple dose vials made of glass or plastic. If administered intravenously, the carrier may be physiological saline or phosphate buffered saline (PBS).

In some embodiments, the present invention refers to a co-administered combination according to the disclosure herein for use in (i) preventing, inhibiting the progression of or treating a brain tumor or (ii) ameliorating at least one symptom associated with a brain tumor in a subject in need thereof. In some embodiments, the present invention relates to a pharmaceutical composition according to the disclosure herein for use in (i) preventing, inhibiting the progression of or treating a brain tumor or (ii) ameliorating at least one symptom associated with a brain tumor in a subject in need thereof.

### 3. METHODS

### 3.1 Methods of Using Co-Administered Combinations

In another aspect, the present invention refers to a method for (i) preventing, inhibiting the progression of or treating a brain tumor or (ii) ameliorating at least one symptom associated with a brain tumor in a subject in need thereof, which comprises administering to the subject a therapeutically effective amount of a co-administered combination comprising (a) at least one glycolysis inhibitor/modulator (e.g., DCA) and (b) at least one prenyltransferase inhibitor (e.g., POH) according to the disclosure. In some embodiments, the subject is human.

In some embodiments, the formulations of the glycolysis inhibitor/modulator (e.g., DCA) and the prenyltransferase inhibitor (e.g., POH) are administered jointly. In some embodiments, the formulations of the glycolysis inhibitor/modulator (e.g., DCA) and the prenyltransferase inhibitor are administered separately. In some embodiments, the formulation of the glycolysis inhibitor/modulator (e.g., DCA) is administered separately before the administration of the formulation of prenyltransferase inhibitor (e.g., POH). In some embodiments, the formulation of the glycolysis inhibitor/modulator (e.g., DCA) is administered separately after the administration of the formulation of prenyltransferase inhibitor (e.g., POH). In some embodiments, the additional active agent is temozolomide. In some embodiments, the glycolysis inhibitor/modulator DCA and the prenyltransferase inhibitor POH are administered concomitantly with temozolomide.

In some embodiments, the formulation of the glycolysis inhibitor/modulator (e.g., DCA) and the formulation of prenyltransferase inhibitor (e.g., POH) are administered via the enteral, parenteral or topical routes of administration. In some embodiments, the formulation of the glycolysis inhibitor/modulator (e.g., DCA) and the formulation of prenyltransferase inhibitor (e.g., POH) are administered using the same route of administration. In some embodiments, the formulation of the glycolysis inhibitor/modulator (e.g., DCA) and the formulation of prenyltransferase inhibitor (e.g., POH) are administered using different routes of administration. In some embodiments, the formulation of the glycolysis inhibitor/modulator (e.g., DCA) is administered orally and the formulation of prenyltransferase inhibitor (e.g., POH) is administered intranasally. In some embodiments, the formulation of the glycolysis inhibitor/modulator (e.g., DCA) is administered intranasally and the formulation of prenyltransferase inhibitor (e.g., POH) is also administered intranasally. In some embodiments, the intranasal administration is conducted by using an atomizer, pipette, soaked pledget, syringe dropper or a combination thereof.

In some embodiments, an additional active agent as described in the disclosure herein (e.g., anticancer agent, anti-glioblastoma agent, palliative agent) is administered separately with the co-administered combination of the present invention. In some embodiments, the additional active agent is administered separately before the formulations of the co-administered combination. In some embodiments, the additional active agent is administered separately at the same time that the formulations of the co-administered combination. In some embodiments, the additional active agent is administered separately after the formulations of the co-administered combination. The order and timing of the administration of the formulations of the co-administered combination and the additional active agent may be adjusted by the skilled person according to the sex, age, and medical condition of the subject.

In some embodiments, the formulation of the glycolysis inhibitor/modulator (e.g., DCA) and the formulation of prenyltransferase inhibitor (e.g., POH) are administered as a single dose form. In some embodiments, the formulation of the glycolysis inhibitor/modulator (e.g., DCA) and the formulation of prenyltransferase inhibitor (e.g., POH) are administered in a multiple unit dosage form.

In some embodiments, the POH dose is administered at least 1, 2, 3, 4, 5 or 6 times a day. In some embodiments, the DCA dose is administered at least 1, 2, 3 or 4 times a day. In some embodiments, the POH dose and the DCA dose are administered at least 1, 2, 3, 4, 5, 6 or 7 times per week. In some embodiments, the POH dose and the DCA dose are administered for at least one or more weeks.

In some embodiments, the POH dose and the DCA dose are administered for one or more years. In some embodiments, the POH dose and the DCA dose are administered during the life of the subject.

In some embodiments, the subject is undergoing chemotherapy, immunotherapy, radiotherapy or a combination thereof. In some embodiments, the subject is administered the co-administered combination of the present invention before undergoing chemotherapy, immunotherapy, radiotherapy or a combination thereof. In some embodiments, the subject is administered the co-administered combination while undergoing chemotherapy, immunotherapy, radiotherapy or a combination thereof. In some embodiments, the subject is administered the co-administered combination after undergoing chemotherapy, immunotherapy, radiotherapy or a combination thereof.

In some embodiments, the present invention refer to the use of a co-administered combination according to the disclosure herein in the manufacture of a medicament for (i) preventing, inhibiting the progression of or treating a brain tumor or (ii) ameliorating at least one symptom associated with a brain tumor in a subject in need thereof.

### 3.2 Methods of Using Pharmaceutical Compositions

In another aspect, the present invention refers to a method for (i) preventing, inhibiting the progression of or treating a brain tumor or (ii) ameliorating at least one symptom associated with a brain tumor in a subject in need thereof, which comprises administering to the subject a therapeutically effective amount of a pharmaceutical composition comprising (a) at least one glycolysis inhibitor/modulator (e.g., DCA) and (b) at least one prenyltransferase inhibitor (e.g., POH) according to the disclosure. In some embodiments, the subject is human.

In some embodiments, the pharmaceutical composition of the present invention is administered via the enteral, parenteral or topical routes of administration. In some embodiments, the pharmaceutical composition is administered intranasally. In some embodiments, the intranasal administration is conducted by using an atomizer, pipette, soaked pledget, syringe dropper or a combination thereof. In some embodiments, the additional active agent is temozolomide. In some embodiments, the glycolysis inhibitor/modulator DCA and the prenyltransferase inhibitor POH are administered jointly or concomitantly with temozolomide.

In some embodiments, an additional active agent as described in the disclosure herein (e.g., anticancer agent, anti-glioblastoma agent, palliative agent) is administered separately with the pharmaceutical composition of the present invention. In some embodiments, the additional active agent is administered separately before the pharmaceutical composition. In some embodiments, the additional active agent is administered separately at the same time that the pharmaceutical composition. In some embodiments, the additional active agent is administered separately after the pharmaceutical composition. The order and timing of the administration of the pharmaceutical composition and the additional active agent may be adjusted by the skilled person according to the sex, age, and medical condition of the subject.

In some embodiments, the pharmaceutical composition of the present invention is administered at least 1, 2, 3, 4, 5 or 6 times a day. In some embodiments, the pharmaceutical composition is administered at least 1, 2, 3, 4, 5, 6 or 7 times per week. In some embodiments, the pharmaceutical composition is administered for at least one or more weeks.

In some embodiments, the pharmaceutical composition is administered for one or more years. In some embodiments, the pharmaceutical composition is administered during the life of the subject.

In some embodiments, the subject is undergoing chemotherapy, immunotherapy, radiotherapy or a combination thereof. In some embodiments, the subject is administered the pharmaceutical composition of the present invention before undergoing chemotherapy, immunotherapy, radiotherapy or a combination thereof. In some embodiments, the subject is administered the pharmaceutical composition while undergoing chemotherapy, immunotherapy, radiotherapy or a combination thereof. In some embodiments, the subject is administered the pharmaceutical composition after undergoing chemotherapy, immunotherapy, radiotherapy or a combination thereof.

In some embodiments, the present invention refers to the use of a pharmaceutical composition according to the disclosure herein in the manufacture of a medicament for (i) preventing, inhibiting the progression of or treating a brain tumor or (ii) ameliorating at least one symptom associated with a brain tumor in a subject in need thereof.

The active agent or agents in the formulations of the co-administered combination or the pharmaceutical composition of the present invention are included in a pharmaceutically acceptable vehicle in an amount sufficient to deliver to a patient a therapeutically effective amount for the desired indication, without causing serious toxic effects in the patient treated. In some embodiments, a dose of an active agent for all of the herein-mentioned conditions is in the range from about 10 ng/kg to 300 mg/kg, 0.1 to 200 mg/kg per day or 0.5 to about 200 mg per kilogram body weight of the recipient/patient per day. A typical topical dosage will range from 0.01-5% wt/wt in a pharmaceutically acceptable vehicle. The active agent is conveniently administered in any suitable unit dosage form, including but not limited to one containing less than 1 mg, about 1 mg to about 3000 mg or about 5 to about 500 mg of active agent per unit dosage form. An oral dosage of about 25 to 250 mg is often convenient. The active agent could be administered to achieve peak plasma concentrations of about 0.00001-30 mM or about 0.1-30 μM.

### 4. KITS

In another aspect, the present invention relates to a kit including one or more containers comprising a therapeutically effective amount of a co-administered combination or a pharmaceutical comprising (a) at least one glycolysis inhibitor/modulator (e.g., DCA) and (b) at least one prenyltransferase inhibitor (e.g., POH) according to the disclosure herein for (i) preventing, inhibiting the progression of or treating a brain tumor or (ii) ameliorating at least one symptom associated with a brain tumor in a subject in need thereof.

In some embodiments, the DCA and POH formulations of the co-administered combination are formulated for enteral, parenteral or topical administration. In some embodiments, the DCA and POH formulations of the co-administered combination are formulated for oral or intranasal administration.

In some embodiments, the pharmaceutical composition (e.g., containing DCA and POH) is formulated for enteral, parenteral or topical administration. In some embodiments, the pharmaceutical composition is formulated for intranasal administration.

In some embodiments, the kit comprises further an atomizer, pipette, soaked pledget, syringe dropper or a combination thereof for administering intranasally the co-administered combinations and pharmaceutical compositions of the present invention.

### 5. EXEMPLARY EMBODIMENTS

The following embodiments further illustrate the scope of the invention:
1. A co-administered combination comprising a therapeutically effective amount of (a) at least one glycolysis inhibitor/modulator and (b) at least one prenyltransferase inhibitor, wherein the co-administered combination is effective for (i) preventing, inhibiting progression or treating a brain tumor or (ii) ameliorating at least one symptom associated with a brain tumor in a subject.
2. The co-administered combination according to embodiment 1, wherein the glycolysis inhibitor/modulator is a direct glycolysis inhibitor or an indirect glycolysis inhibitor/modulator.
3. The co-administered combination according to any of the preceding embodiments, wherein the direct glycolysis inhibitor is selected from the group consisting of 2-deoxy-D-glucose, 3-bromopyruvate, 3PO, FX11, galloflavin, lonidamine, oxamate, PFK-15, SF2312, shikonin, and WZB117 or a combination thereof.
4. The co-administered combination according to any of the preceding embodiments, wherein the direct glycolysis inhibitor is 2-deoxy-D-glucose.
5. The co-administered combination according to any of the preceding embodiments, wherein the indirect glycolysis inhibitor/modulator is selected from the group consisting of apigenin, berberine, dichloroacetate, epigallocatechin-3-gallate (EGCG), metformin, quercetin, resveratrol, and vitamin C or a combination thereof.
6. The co-administered combination according to any of the preceding embodiments, wherein the indirect glycolysis inhibitor/modulator is dichloroacetate, metformin or a combination thereof.
7. The co-administered combination according to any of the preceding embodiments, wherein the prenyltransferase inhibitor is a direct farnesyltransferase inhibitor or a direct geranylgeranyltransferase inhibitor.
8. The co-administered combination according to any of the preceding embodiments, wherein the direct farnesyltransferase inhibitor is selected from the group consisting of BMS-214662, BMS-214662 analogs, 6-gingerol, 8-gingerol, gliotoxin, lonafarnib, lonafarnib analogs, manumycin A, manumycin A analogs, perillyl alcohol, S-(−)-perillyl alcohol, R115777, R115777 derivatives, salirasib, tipifarnib, and tipifarnib analogs or a combination thereof.
9. The co-administered combination according to any of the preceding embodiments, wherein the direct farnesyltransferase inhibitor is 6-gingerol, lonafarnib, perillyl alcohol or a combination thereof.
10. The co-administered combination according to any of the preceding embodiments, wherein the direct geranylgeranyltransferase inhibitor is selected from the group consisting of 3-PEHPC, GGTI-298, GGTI-2133, GGTI-P4, L-778123, and NSC-697923 or a combination thereof.
11. The co-administered combination according to any of the preceding embodiments, wherein the prenyltransferase inhibitor is an indirect farnesyltransferase inhibitor or an indirect geranylgeranyltransferase inhibitor.
12. The co-administered combination according to any of the preceding embodiments, wherein the indirect farnesyltransferase inhibitor or the indirect geranylgeranyltransferase inhibitor is selected from the group consisting of a zoledronic acid and simvastatin or a combination thereof.
13. The co-administered combination according to any of the preceding embodiments, wherein the glycolysis inhibitor/modulator is 2-deoxy-D-glucose, dichloroacetate, metformin or a combination thereof and the prenyltransferase inhibitor is 6-gingerol, lonafarnib, perillyl alcohol or a combination thereof.
14. The co-administered combination according to any of the preceding embodiments, wherein the glycolysis inhibitor/modulator is dichloroacetate and the prenyltransferase inhibitor is perillyl alcohol.
15. The co-administered combination according to any of the preceding embodiments, wherein the therapeutically effective amounts of 2-deoxy-D-glucose, dichloroacetate, metformin, 6-gingerol, lonafarnib, perillyl alcohol or a combination thereof are synergistic.
16. The co-administered combination according to any of the preceding embodiments, wherein the brain tumor is brain cancer.
17. The co-administered combination according to any of the preceding embodiments, wherein the brain cancer is selected from the group consisting of astrocytoma, brain stem glioma, chordoma, cranial base tumor craniopharyngioma, diffuse intrinsic pontine glioma, ependymoma, glioblastoma, glioblastoma multiforme, glioma, haemangioblastoma, lymphoma, medulloblastoma, meningioma, and glioneuronal tumors, oligodendroglioma, pineal tumors, pituitary adenoma, rhabdoid tumors vestibular secondary brain tumors due to metastasis, and schwannoma or a combination thereof.
18. The co-administered combination according to any of the preceding embodiments, wherein the brain cancer is glioblastoma multiforme.
19. The co-administered combination according to any of the preceding embodiments, wherein the glycolysis inhibitor/modulator, the prenyltransferase inhibitor or both, further comprise a pharmaceutically acceptable carrier or excipient.
20. The co-administered combination according to any of the preceding embodiments, wherein the co-administered combination comprises at least one additional active agent.
21. The co-administered combination according to any of the preceding embodiments, wherein the additional active agent comprises an anticancer agent, anti-excitotoxic/calcium modulator, anti-inflammatory/microglial modulator, mitochondrial protectant, neuroprotective/antioxidant agent, neurotrophic/synaptic support agent, other neuroprotective/metabolic modulating medication, a palliative agent or a combination thereof.
22. The co-administered combination according to any of the preceding embodiments, wherein the anticancer agent is an anti-glioblastoma agent.
23. The co-administered combination according to any of the preceding embodiments, wherein the anti-glioblastoma agent comprises temozolomide, lomustine, bevacizumab or a combination thereof.
24. The co-administered combination according to any of the preceding embodiments, wherein the palliative agent is selected from the group consisting of codeine, dexamethasone, levothyroxine, octanoic acid, ondansetron, tramadol, vitamin B3, vitamin D or a combination thereof.
25. The co-administered combination according to any of the preceding embodiments, wherein the glycolysis inhibitor/modulator and the prenyltransferase inhibitor are formulated for enteral, parenteral or topical administration.
26. The co-administered combination according to any of the preceding embodiments, wherein the glycolysis inhibitor/modulator and the prenyltransferase inhibitor are formulated for co-administration via the same or different routes of administration.
27. The co-administered combination according to any of the preceding embodiments, wherein the glycolysis inhibitor/modulator dichloroacetate is formulated for oral or intranasal administration and the prenyltransferase inhibitor perillyl alcohol is formulated for intranasal administration.
28. The co-administered combination according to any of the preceding embodiments, wherein perillyl alcohol is formulated at a dose of about 3 mg/kg to about 20 mg/kg.
29. The co-administered combination according to any of the preceding embodiments, wherein dichloroacetate is formulated at a dose of about 2 mg/kg to about 20 mg/kg.
30. The co-administered combination according to any of the preceding embodiments, wherein the subject is human.
31. A method for (i) preventing, inhibiting the progression of or treating a brain tumor or (ii) ameliorating at least one symptom associated with a brain tumor in a subject in need thereof, which comprises administering to the subject a therapeutically effective amount of a co-administered combination comprising (a) at least one glycolysis inhibitor/modulator and (b) at least one prenyltransferase inhibitor.
32. The method according to embodiment 31, wherein the co-administered combination is according to any one of embodiments 1-30.
33. The method according to any one of embodiments 31-32, wherein the perillyl alcohol dose is administered at least 1, 2, 3, 4, 5 or 6 times a day.
34. The method according to any one of embodiments 31-33, wherein the dichloroacetate dose is administered at least 1, 2, 3 or 4 times a day.
35. The method according to any one of embodiments 31-34, wherein the perillyl alcohol dose and the dichloroacetate dose are administered at least 1, 2, 3, 4, 5, 6 or 7 times per week.
36. The method according to any one of embodiments 31-35, wherein the perillyl alcohol dose and the dichloroacetate dose are administered for at least one or more weeks.
37. The method according to any one of embodiments 31-36, wherein the perillyl alcohol dose and the dichloroacetate dose are administered for one or more years.
38. The method according to any one of embodiments 31-37, wherein the perillyl alcohol dose and the dichloroacetate dose are administered during the life of the subject.
39. The method according to any one of embodiments 31-38, wherein the subject is undergoing chemotherapy, immunotherapy, radiotherapy or a combination thereof.
40. The method according to any one of embodiments 31-39, wherein the subject is human.
41. A pharmaceutical composition comprising a therapeutically effective amount of (a) at least one glycolysis inhibitor/modulator and (b) at least one prenyltransferase inhibitor wherein the pharmaceutical composition is effective for (i) preventing, inhibiting the progression of or treating a brain tumor or (ii) ameliorating at least one symptom associated with a brain tumor in a subject.
42. The pharmaceutical composition according to embodiment 41, wherein the glycolysis inhibitor/modulator is a direct glycolysis inhibitor or an indirect glycolysis inhibitor/modulator.
43. The pharmaceutical composition according to any one of embodiments 41-42, wherein the direct glycolysis inhibitor is selected from the group consisting of 2-deoxy-D-glucose, 3-bromopyruvate, 3PO, FX11, galloflavin, lonidamine, oxamate, PFK-15, SF2312, shikonin, and, WZB117 or a combination thereof.
44. The pharmaceutical composition according to any one of embodiments 41-43, wherein the direct glycolysis inhibitor is 2-deoxy-D-glucose.
45. The pharmaceutical composition according to any one of embodiments 41-44, wherein the indirect glycolysis inhibitor/modulator is selected from the group consisting of apigenin, berberine, dichloroacetate, epigallocatechin-3-gallate (EGCG), metformin, quercetin, resveratrol, and vitamin C or a combination thereof.
46. The pharmaceutical composition according to any one of embodiments 41-45, wherein the indirect glycolysis inhibitor is dichloroacetate, metformin or a combination thereof.
47. The pharmaceutical composition according to any one of embodiments 41-46, wherein the prenyltransferase inhibitor is a direct farnesyltransferase inhibitor or a direct geranylgeranyltransferase inhibitor.
48. The pharmaceutical composition according to any one of embodiments 41-47, wherein the direct farnesyltransferase inhibitor is selected from the group consisting of BMS-214662, BMS-214662 analogs, 6-gingerol, 8-gingerol, gliotoxin, lonafarnib, lonafarnib analogs, manumycin A, manumycin A analogs, perillyl alcohol, S-(−)-perillyl alcohol, R115777, R115777 derivatives, salirasib, tipifarnib, and tipifarnib analogs or a combination thereof.
49. The pharmaceutical composition according to any one of embodiments 41-48, wherein the direct farnesyltransferase inhibitor is 6-gingerol, lonafarnib, perillyl alcohol or a combination thereof.
50. The pharmaceutical composition according to any one of embodiments 41-49, wherein the direct geranylgeranyltransferase inhibitor is selected from the group consisting of 3-PEHPC, GGTI-298, GGTI-2133, GGTI-P4, L-778123, and NSC-697923 or a combination thereof.
51. The pharmaceutical composition according to any one of embodiments 41-50, wherein the prenyltransferase inhibitor is an indirect farnesyltransferase inhibitor or an indirect geranylgeranyltransferase inhibitor.
52. The pharmaceutical composition according to any one of embodiments 41-51, wherein the indirect farnesyltransferase inhibitor or the indirect geranylgeranyltransferase inhibitor is selected from the group consisting of a zoledronic acid and simvastatin or a combination thereof.
53. The pharmaceutical composition according to any one of embodiments 41-52, wherein the glycolysis inhibitor/modulator is 2-deoxy-D-glucose, dichloroacetate, metformin or a combination thereof and the prenyltransferase inhibitor is 6-gingerol, lonafarnib, perillyl alcohol or a combination thereof.
54. The pharmaceutical composition according to any one of embodiments 41-53, wherein the glycolysis inhibitor/modulator is dichloroacetate and the prenyltransferase inhibitor is perillyl alcohol.
55. The pharmaceutical composition according to any one of embodiments 41-54, wherein the therapeutically effective amounts of 2-deoxy-D-glucose, dichloroacetate, metformin, 6-gingerol, lonafarnib, perillyl alcohol or a combination thereof are synergistic.
56. The pharmaceutical composition according to any one of embodiments 41-55, wherein the brain tumor is brain cancer.
57. The pharmaceutical composition according to any one of embodiments 41-56, wherein the brain cancer is selected from the group consisting of astrocytoma, brain stem glioma, chordoma, cranial base tumor craniopharyngioma, diffuse intrinsic pontine glioma, ependymoma, glioblastoma, glioblastoma multiforme, glioma, haemangioblastoma, lymphoma, medulloblastoma, meningioma, neuronal and glioneuronal tumors, oligodendroglioma, pineal tumors, pituitary adenoma, rhabdoid tumors vestibular secondary brain tumors due to metastasis, and schwannoma or a combination thereof.
58. The pharmaceutical composition according to any one of embodiments 41-57, wherein the brain cancer is glioblastoma multiforme.
59. The pharmaceutical composition according to any one of embodiments 41-58, further comprising a pharmaceutically acceptable carrier or excipient.
60. The pharmaceutical composition according to any one of embodiments 41-59, further comprising at least one additional active agent.
61. The pharmaceutical composition according to any one of embodiments 41-60, wherein the additional active agent comprises an anticancer agent, anti-excitotoxic/calcium modulator, anti-inflammatory/microglial modulator, mitochondrial protectant, neuroprotective/antioxidant agent, neurotrophic/synaptic support agent, other neuroprotective/metabolic modulating medication, palliative agent or a combination thereof.
62. The pharmaceutical composition according to any one of embodiments 41-61, wherein the anticancer agent is an anti-glioblastoma agent.
63. The pharmaceutical composition according to any one of embodiments 41-62, wherein the anti-glioblastoma agent comprises temozolomide, lomustine, bevacizumab or a combination thereof.
64. The pharmaceutical composition according to any one of embodiments 41-63, wherein the palliative agent is selected from the group consisting of codeine, dexamethasone, levothyroxine, octanoic acid, ondansetron, tramadol, vitamin B3, vitamin D or a combination thereof.
65. The pharmaceutical composition according to any one of embodiments 41-64, wherein the pharmaceutical composition is formulated for enteral, parenteral or topical administration.
66. The pharmaceutical composition according to any one of embodiments 41-65, wherein the pharmaceutical composition is formulated for intranasal administration.
67. The pharmaceutical composition according to any one of embodiments 41-66, wherein perillyl alcohol is formulated at a dose of about 3 mg/kg to about 20 mg/kg.
68. The pharmaceutical composition according to any one of embodiments 41-67, wherein dichloroacetate is formulated at a dose of about 2 mg/kg to about 20 mg/kg.
69. The pharmaceutical composition according to any one of embodiments 41-68, wherein the subject is human.
70. A method for (i) preventing, inhibiting the progression of or treating a brain tumor or (ii) ameliorating at least one symptom associated with a brain tumor in a subject in need thereof, which comprises administering to the subject a therapeutically effective amount of a pharmaceutical composition comprising (a) at least one glycolysis inhibitor/modulator and (b) at least one prenyltransferase inhibitor.
71. The method according to embodiment 70, wherein the pharmaceutical composition is according to any one of embodiments 41-69.
72. The method according to any one of embodiments 70-71, wherein the pharmaceutical composition is administered at least 1, 2, 3, 4, 5 or 6 times a day.
73. The method according to any one of embodiments 70-72, wherein the pharmaceutical composition is administered at least 1, 2, 3, 4, 5 or 7 times per week.
74. The method according to any one of embodiments 70-73, wherein the pharmaceutical composition is administered for at least one or more weeks.
75. The method according to any one of embodiments 70-74, wherein the pharmaceutical composition is administered for at least one or more years.
76. The method according to any one of embodiments 70-75, wherein the pharmaceutical composition is administered during the life of the subject.
77. The method according to any one of embodiments 70-76, wherein the subject is undergoing chemotherapy, immunotherapy, radiotherapy or a combination thereof.
78. The method according to any one of embodiments 70-77, further comprising at administering the pharmaceutical concurrently with at least one additional active agent according to any one of embodiments 61-64.
79. The method according to any one of embodiments 70-78, wherein the subject is human.
80. A kit including one or more containers comprising a therapeutically effective amount of a co-administered combination according to any one of embodiments 1-30 or a pharmaceutical according to any one of embodiments 41-69 for (i) preventing, inhibiting the progression of or treating a brain tumor or (ii) ameliorating at least one symptom associated with a brain tumor in a subject in need thereof.
81. A co-administered combination according to any one of embodiments 1-40 for use in (i) preventing, inhibiting the progression of or treating a brain tumor or (ii) ameliorating at least one symptom associated with a brain tumor in a subject in need thereof.
82. A pharmaceutical composition according to any one of embodiments 41-79 for use in (i) preventing, inhibiting the progression of or treating a brain tumor or (ii) ameliorating at least one symptom associated with a brain tumor in a subject in need thereof.
83. Use of a co-administered combination according to any one of embodiments 1-30 in the manufacture of a medicament for (i) preventing, inhibiting the progression of or treating a brain tumor or (ii) ameliorating at least one symptom associated with a brain tumor in a subject in need thereof.
84. Use of a pharmaceutical composition according to any one of embodiments 41-69 in the manufacture of a medicament for (i) preventing, inhibiting the progression of or treating a brain tumor or (ii) ameliorating at least one symptom associated with a brain tumor in a subject in need thereof.

### Example 1

### Experiences of GBM Patients with the Combination of POH and DCA

Various patients with GBM employed the combinations of invention and the methods for using them. A description of the experiences of these patients with the combinations and methods of the invention follows:

Patient-1: female, 26 years old, 3.5 cm diameter, diagnosed with occipital tumor GBM IV, isocitrate dehydrogenase (IDH) and 06-methylguanine-DNA grade methyltransferase (MGMT) promoter wild types. Patient-1 was a no-option patient with a forecast of three to twelve months of survival. When GBM appeared, Patient-1 underwent a biopsy surgery, then radiotherapy combined with low dosage temozolomide, then temozolomide high doses, with no clinical, nor MRI positive results. She had tumor growth visible at the end of temozolomide (TMZ) treatment and suffered a second partial resection surgery. Patient-1's clinical condition was poor, suffering tonic-clonic seizures ranging from one to nearly twenty per day. Some of seizures were very severe and refractory.

Patient-1 suffered at least five different induced coma periods as the only way to stop some of the seizures. She also had a severe hemiplegia event, as well as Cushing syndrome, due to the high doses of dexamethasone applied to contain the inflammation produced by the tumor. She received up to three different antiepileptic drugs at maximum dosage at the same time, which were changed with her evolution (e.g., levetiracetam, phenytoin, valproic acid, and lacosamide). Seizures were refractory to all the treatments received. She received different sublingual or intra-anal benzodiazepines to control seizures once started, with high success. The use of benzodiazepines concomitant with seizures eliminated the need to induce coma.

Tramadol, codeine, and many other anti-pain products were used for headaches, and extreme bone pain caused by bone infarcts secondary to the excessive use of dexamethasone. Ondansetron and other antiemetics were necessary in some periods, as well as vitamin D and levothyroxine, the former because thyroid was severely affected by the radiotherapy applied.

Patient-1 initiated POH treatment after the second surgery, from month 13-15 of survival, with no other oncologic treatment, at 55 mg/dose TID (three times per day) via inhalation over a period of about 15 minutes each application.

MRI tracking indicated that the tumor stopped growing at first, for a period of about 6 months, and then slowly and continuously reduced its size. The active regions of the tumor disappeared completely after 4 years of treatment with scars remaining visible by MRI in the brain tissue.

Clinic manifestations of the tumor gradually tapered off and disappeared. Patient-1 stopped taking most of the medicines mentioned above. She continued with levetiracetam only and taking sublingual lorazepam (Trapax) when seizures appeared. Seizures were the most evident clinical manifestation that stayed for the whole period from the beginning of the disease up to the death of the Patient-1, although their intensity and frequency decreased compared to the first two years. All other symptoms such as vomiting, headache, bone pain, and paralysis disappeared completely or almost completely. The patient complained of a persistent minor difficulty in the precision movements of the fingers. Quality of life (QoL) dramatically improved and maintained for many years, with a Karnofsky index of at least 90%.

In year 4 of the disease, Patient-1 became pregnant and delivered a healthy boy after a completely normal period of pregnancy.

Patient-1 developed depression in year 7 of the disease attributed to familial issues. GBM became partially resistant to POH almost simultaneously. Tumor grew very quickly and in 3 months invaded not only the left hemisphere of the brain, but part of the right hemisphere, the corpus callosum and the cerebellum, too.

Some of the former clinical manifestations returned, plus lack of parallelism in the eyes because of cerebellum invasion, which provoked double vision and equilibrium troubles. Hemiplegia appeared strongly again.

Patient-1 was declared again as a no-option patient, with a maximum of three months of survival. An additional surgery was performed to remove part of the tumor. Recovery was extremely difficult and painful.

Patient-1 started taking DCA 333 mg twice daily (BID) in conjunction with POH 55 mg three times daily (TID), by mouth. Patient-1 also used octanoic acid, 200 mg, BID, to avoid neuropathy secondary to the DCA treatment.

Unexpectedly, Patient-1 showed steady signs of improvement and experienced a dramatic improvement in QoL from year 8 to year 10 of the disease, when the tumor advanced slowly again, and caused difficulties to swallow. She had food and liquid entering in the lungs because of this, and by the year 10 and 2 months of the disease, contracted pneumonia because of lung aspirations. At this stage, all treatments were stopped due to the deteriorating condition of the patient.

Patient-1 passed after 10 years, 4 months and 21 days from the initial diagnosis of GBM. Significantly, the typical life span for patients with Patient-1's genotype is 10.4 months, with a low QoL. Patient-1 survived over 10 years with particularly good QOL, most of the time. During the patient's final years, the combined use of DCA and POH resulted in the unexpected extension of her expected survival from 3 months to 3 years and 6 months; an almost impossible result, considering the advanced stage and degree of tumor spreading by the time POH and DCA were applied contemporarily.

Patient-2: male, 60 years old, diagnosed with GBM with wild type IDHs and MGMT. He was treated with resection surgery followed by radiotherapy and chemotherapy with TMZ.

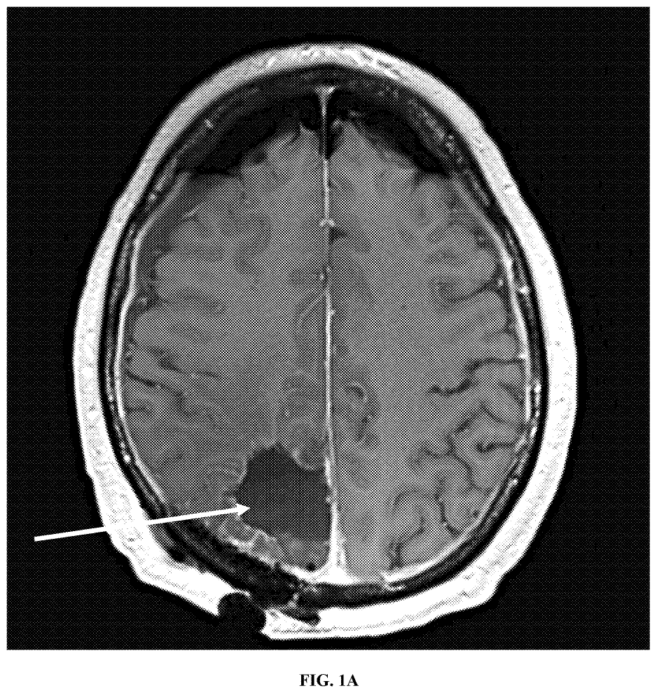

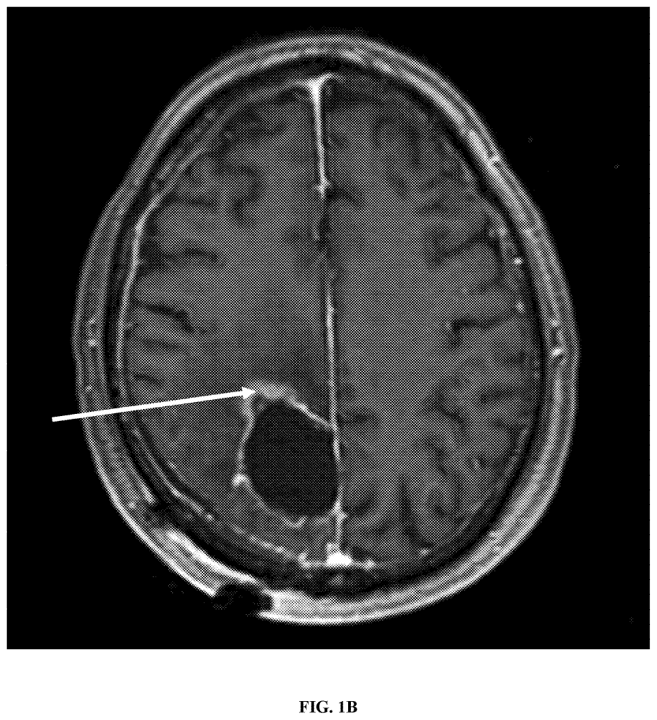

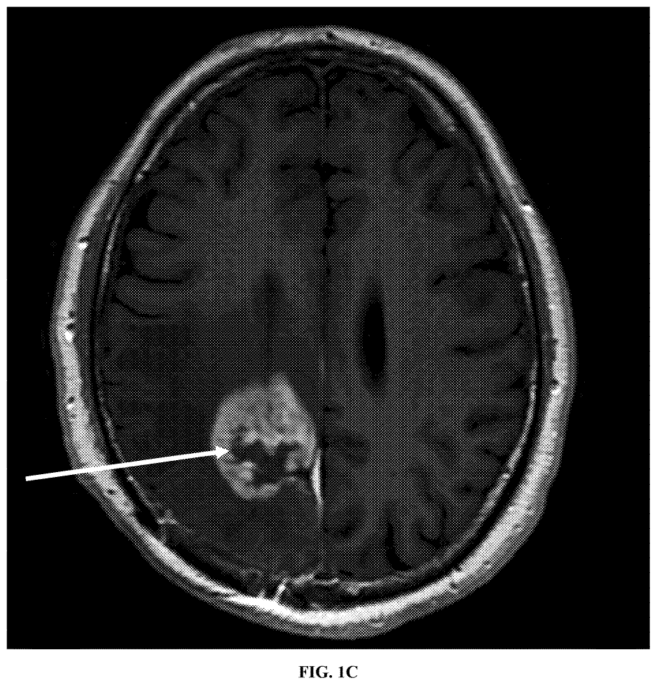

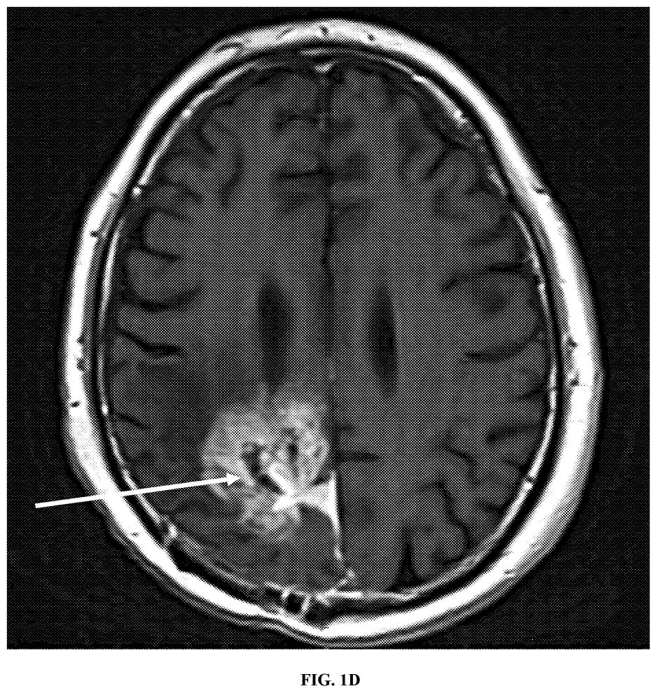

The tumor progressed by month 10 and a new resection surgery was done on month 11. MRI showed an excellent result, with no active areas of tumor visible in the image (FIG. 1A). Fifteen days later the tumor was already present on all the borders of the resection area (i.e., brighter areas around periphery resected area, white arrow; FIG. 1B).

Forty-five days after the surgical intervention, the active tumor had already invaded most of the resection area, with a diameter of 4.8 cm occupied in 30 days (FIG. 1C). Patient-2 was given a three-month life expectancy with no viable treatment options.

Fourteen days after the 4.8 cm diameter image Patient-2 started taking POH, at 55 mg/dose TID via nasally. After several weeks, the dose was increased to 144 mg/dose and the patient started taking also DCA orally BID at 333 mg/dose. Patient-2 also began taking octanoic acid, a neuroprotector, as a palliative strategy to avoid any DCA side effects.

Sixty days after the 4.8 cm image, and 46 days after starting taking POH/DCA treatment, a new MRI was performed. Unexpectedly, the tumor was only 5.2 cm diameter (i.e., patient started taking POH/DCA 2 weeks after the previous image was taken, thus, the 4 mm increase must be attributed to time when the patient did not take POH/DCA) (FIG. 1D).

Patient-2 maintained a relatively high QoL and no new clinical manifestations appeared up to the end of his life, 5.5 months after starting taking POH/DCA.

An MRI showed a small progress of the tumor. Patient-2 suffered severe depression and stopped taking any medicine, including dexamethasone, which was critical for avoiding any inflammation caused by the pressure exerted by the tumor mass in the brain. He died 3 days later, likely due to high intracranial pressure.

Patient-2 had a 3-month survival forecast when he started taking POH/DCA. With the use of POH/DCA, patient survival unexpectedly increased to 5.5 months, with no clinical progression, and maintaining a good QoL.

Patient-3:70 years old, male, diagnosed with GBM wild type IDH and MGMT, a non-surgical 3 cm diameter tumor with a ki-67 of 55% (i.e., rapid growth). Patient-3's tumor was biopsied and then radiotherapy plus. TMZ therapy started at a low dose and increased subsequently to a high dose. POH was given in parallel with TMZ. Sole clinical manifestation was a loss of consciousness for maximum 2 minutes once or twice a day. Had Karnofsky index of almost 100% during the entire process. Patient-3 had a six-month survival forecast.

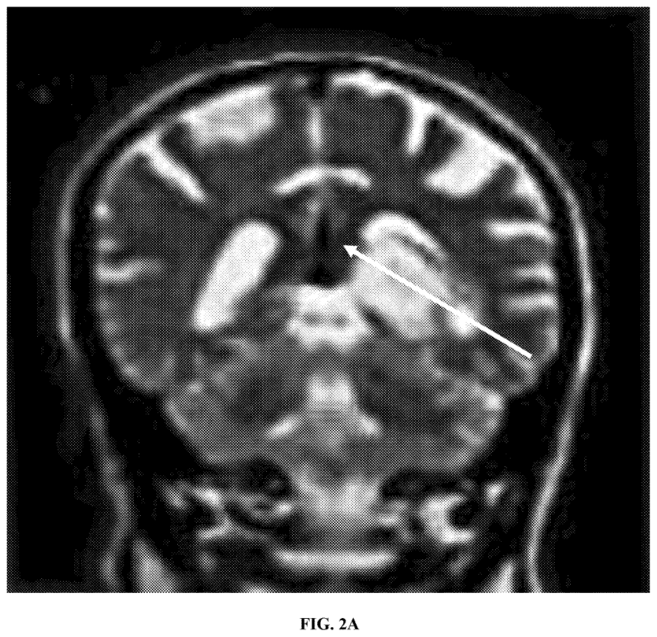

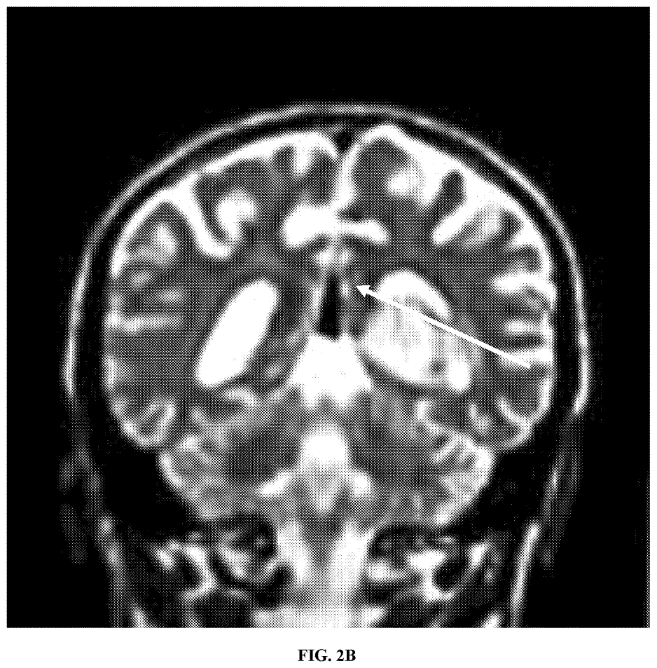

Three months later he started treatment with POH, TID via nasal administration, the dose was increased from 55 mg/dose up to 144 mg/dose in a few weeks. DCA was taken orally at the same time BID at a dose of 333 mg, along with octanoic acid as a neuroprotector. MRI image of the moment the treatment started is seen in FIG. 2A.

Fifty-five days later another MRI was performed (FIG. 2B). The POH/DCA combination showed unexpectedly excellent results, without any progression of the disease, which had been advancing very rapidly before the treatments.

A new MRI performed three months later showed, even more unexpectedly, no signs of any disease progression. The patient maintained a high QoL during this time but complained about hearing continuous noise. Then, Patient-3 stopped taking POH/DCA and died a few days later, most likely due to brain hemorrhage provoked by the internal pressure caused by the tumor.

In summary, the co-administration of POH and DCA prolonged both the survival and QOL for the glioblastoma patients, without any relevant adverse effects. Extension of life expectancy was due to prevention of tumor evolution and growth even when the treatment was started at an advanced stage.

### Example 2

### In Vitro 2-D and 3-D Assays with POH-DCA Combinations

Several studies were performed for evaluating the antitumor activity of LMP01, a novel therapeutic candidate combining POH and DCA, in aggressive GBM models, to lay the scientific foundations for its potential clinical translation for GBM management. The experimental objectives involved a series of 2D and 3D in vitro approaches assessing LMP01 direct cytostatic activity on different human GBM cell lines, in comparison to POH and DCA monotherapies, and TMZ as a reference standard-of-care treatment. The following materials were used:

POH (MW 152.23 g/mol, Sigma Aldrich, USA). Considering the maximum non-toxic dose for cells (1% DMSO), the compound was diluted using dimethyl sulfoxide (DMSO) to reach final working concentrations.

DCA (MW 150.92 g/mol, Curaltus Ltd, Vilnius, LT). The drug was first solubilized using sterile water to generate concentrated stocks and subsequent dilutions using sterile water were conducted to generate final working concentrations.

TMZ (MW 194.151 g/mol, Sigma Aldrich, USA). The compound was initially dissolved in DMSO to generate concentrated stocks. Subsequent dilutions using DMSO were conducted to attain final working concentrations, without reaching cytotoxic concentration for cells.

LN229 (ATCC CRL-2611) is an extremely aggressive human GBM cell model, widely used in biomedical research. It was isolated in 1979 from the right frontal parieto-occipital cortex of a white, 60-year-old, female patient with GBM. It has an epithelial-like morphology and it is tumorigenic in nude mice. It bears a mutation in p53 (TP53) and possible homozygous deletions in the p16 and p14ARF tumor suppressor genes.

LN18 (ATCC CRL-2610) is a wild-type human brain tumor glioblastoma cell line used in neuroscience research and has an epithelial-like morphology. It was isolated in 1976 from the right temporal lobe of a white, 65-year-old, male patient with glioblastoma.

T98G (ATCC CRL-1690) is a fibroblast-like cell line that was isolated from the brain of a glioblastoma multiforme white, 61-year-old, male patient. It is commonly used in research to study brain cancer and related molecular mechanisms.

LN229 and LN18 were grown using Dulbecco's modified Eagle's medium (DMEM) culture medium (Gibco, USA) and T98G was grown using DMEM: F12 (1:1) medium (Gibco, USA), in all cases cells were supplied with 10% fetal bovine serum (FBS) (Natocor, Córdoba, AR), 80 μg/mL gentamicin, and 2 mM glutamine, in monolayer culture, at 37 °C in a humidified atmosphere of 5% CO₂. Cells were harvested using a trypsin/EDTA solution (Thermo Fisher Scientific Inc., USA) diluted in PBS and routinely assessed for mycoplasma.

### 1. 2D Cell Model Determination of Antitumor Effect and Synergism for POH and DCA

A high-density log-phase proliferation assay was used to evaluate GBM cellular growth. Briefly, LN229, LN18, and T98G cells were plated in 96 multi-well plate format at a density of 2-3×10\hat{ }3 cells per 100 μL of complete DMEM or DMEM/F12 medium. After 24 h, cells were exposed to POH or DCA (range concentrations of 0.1-2 mM and 5-100 mM, respectively). 72 h later exponentially growing cell cultures were fixed for 10 min with methanol and then stained with 0.5% crystal violet solution for another 10 min. Following a washing step, fixed cultures were dried overnight. Quantification for the crystal violet method was performed by absorbance measurement (2=595 nm) after adding an ethanol/acetic acid 3:1 solution. Values are presented as a percentage of control. 6-12 wells per experimental group per independent replicate. The tests were performed by duplicate (independent operators). High resolution photographs were taken from different representative wells from each experimental group.

To investigate a possible synergistic effect by combining both POH and DCA, different high-density cell proliferation assays were conducted in the exponential phase for 72 h. The potential cooperative effects between drug pairs were evaluated using a combinational matrix design. For this purpose, LN229 cells were seeded at a density of 2×10\hat{ }3 cells per well, allowed to attach overnight, and then treated using optimal (IC₅₀) and suboptimal (IC₁₀ and IC₂₅) concentrations of the drugs.

Outlier identification, analysis of combination indexes, dose-response curve shifts, isobolograms, dose reduction indexes, and synergy scores, the statistical packages PRISM GraphPad, Compusyn, and SynergyFinder were used for subsequent comparative statistical analysis between experimental groups.

### 2. Tumor Cell Migration Inhibition Test

Transwell® migration assays were conducted to evaluate GBM cell chemotaxis. After overnight starvation (0% FBS), 7.5×10\hat{ }4 LN229 cells were seeded into 8 μm-pore inserts (JetBiofil, Guangzhou, CN) in serum-free DMEM and POH (0.5 mM), DCA (30 mM) or LMP01 (POH 0.5 mM plus DCA 30 mM). Lower chambers were filled with DMEM containing FBS as chemoattractant.

### 3. Tumor Cell Migration Inhibition Test

After 16 h-incubation with LMP01, cells present in the upper surface of the membranes were removed with cotton swabs and cells that migrated through the insert and attached to its lower surface were fixed, stained, and washed. Migrating cells in 4 randomly selected X200-high power field (HPF) per insert were quantified using the open source QuPath Software v0.3.0, and normalized to control (QuPath, Quantitative Pathology and Bioimage Analysis, University of Edinburgh, GB, qupath.github.io). Performed by independent operators, 12-16 X200-HPF were obtained per experimental group, per independent replicate.

### 4. 3D Cell Model Antitumor Effect

For spheroid formation, the hanging drop method was used. Briefly, 20 μL of complete medium containing 5000 LN229 cells was seeded on the top cover of a 100 mm culture plate and allowed to form a spheroid. After 72 h, the resulting cell structures were transferred to a 96-well plate coated with 50 μL of 1.5% agar to prevent cell adhesion to the plastic. Approximately 7 days after transfer, once the spheroids showed adequate sphericity and a minimum diameter of 200 μm, they were randomly separated into groups and treatment with 0.75-2 mM POH, 15-60 mM DCA or LMP01 (assessing different POH+DCA combos) started. The complete medium, with or without treatment, was renewed twice a week for 10-14 days. Spheroids were photographed twice weekly using a high-resolution optical microscope (Leica Microsystems, Wetzlar, DE) to track each individual spheroid and to calculate its volume throughout the experiment, relative to the initial day of treatment (values at day 0 were taken as 100%). Spheroid diameters were quantified using ImageJ 1.5j8 software (NIH, Maryland, USA, imagej.nih.gov and were subsequently converted to volume using the following formula: 4/3*3.14*(diameter/2)\hat{ }3.

Additionally, the impact of the monotherapies and the LMP01 combination therapy was also assessed on 3D LN18 cultures, following the same criteria for spheroid formation and subsequent analysis as explained above with the LN229 model. In contrast to LN229, which forms rapidly growing spheroids, 3D cultures of the human GBM cell line LN18 rapidly decline in size, losing cellular cohesion and integrity. Despite this limitation, the therapeutic impact of LMP01 was evaluated in an ancillary manner on the dynamics of LN18 3D culture. Combinatorial studies of LMP01 in addition to TMZ.

Once LMP01 was evaluated in both LN229 and LN18 cellular models, concomitant studies of LMP01 in addition to TMZ were conducted in 3D spheroid and chemotaxis models. For 3D spheroid growth, following the same protocol described above, once the spheroids showed adequate sphericity and diameter, they were randomly separated into groups and treatment with LMP01 (POH 0.75 mM and DCA 15 mM), TMZ 50 μM (Berte N, et al., Oncotarget 2016; 7(41): 67235-67250) and its combination, for the LN229 model, and LMP01 (POH 0.5 mM and DCA 30 mM), TMZ 50 μM and its combination, for the LN18 model, was started. The complete medium, with or without treatment, was renewed twice a week for 10-14 days and spheroids were photographed twice weekly. The subsequent analysis was conducted following the same criteria mentioned above. Additionally, Transwell® migration assays were conducted, as previously described.

After overnight starvation, 7.5×10\hat{ }4 LN229 cells were seeded into 8 μm-pore inserts in serum-free DMEM and LMP01 (POH 0.5 mM and DCA 30 mM), TMZ 200 μM and its combination. Lower chambers were filled with DMEM containing FBS as chemoattractant and after 16 h treatment cells were fixed, stained and washed. Migrating cells in 4 randomly selected X200-high power field (HPF) per insert were quantified using the open source QuPath Software v0.3.0 and normalized to control. In vitro assay determinations were conducted from at least 2 or 3 independent experiments, unless stated otherwise, with a variable number of replicates per experiment type ranging from 3-12 per experimental group.

Statistical analyses were mainly performed using GraphPad Prism 8.0V software, (GraphPad Prism Software, USA). Distribution of data and descriptive statistics were first assessed. Outliers were detected and removed (Prism calculator). For comparisons among 3 or more experimental groups, ANOVA or its nonparametric equivalent, the Kruskal-Wallis test, were utilized. Subsequently, tests such as Dunnett's test, Tukey's test, comparison of 95% confidence intervals (CI) for the mean (ANOVA) or Dunn's test (Kruskal-Wallis) were employed. For comparisons between 2 groups, either the Student t-test or the Mann-Whitney test were used, depending on the parametric or non-parametric distribution of values, respectively. For the calculation of the half inhibitory concentration (IC₅₀), nonlinear regressions were applied, based on optimal curve-fitting. Differences were considered statistically significant at a level of p<0.05; *p<0.05; **p<0.01; ***p<0.001; ****p<0.0001.

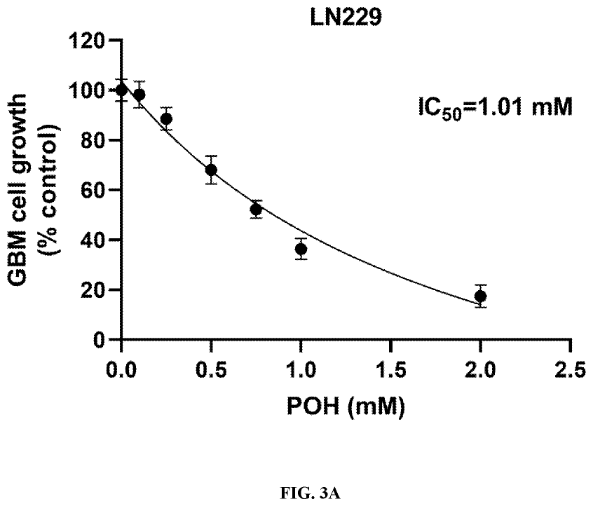

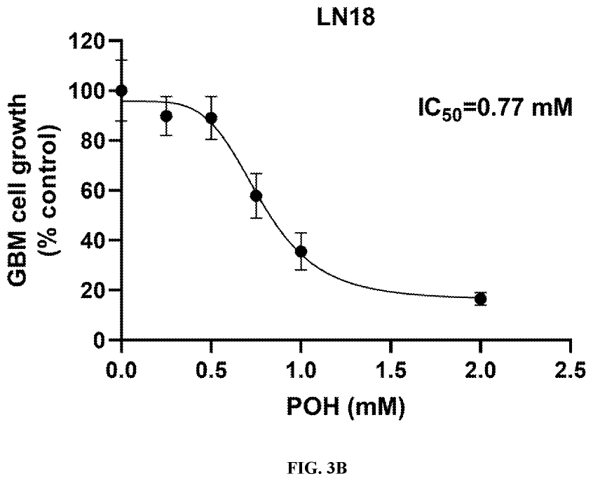

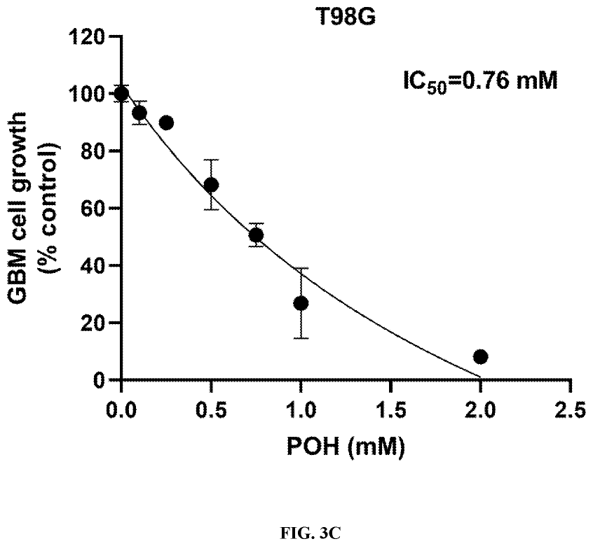

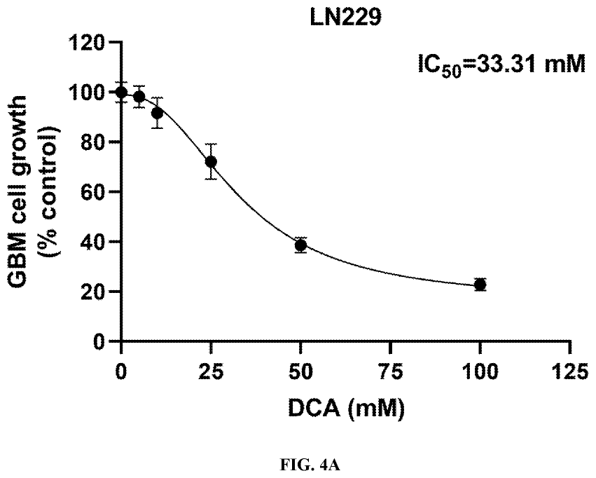

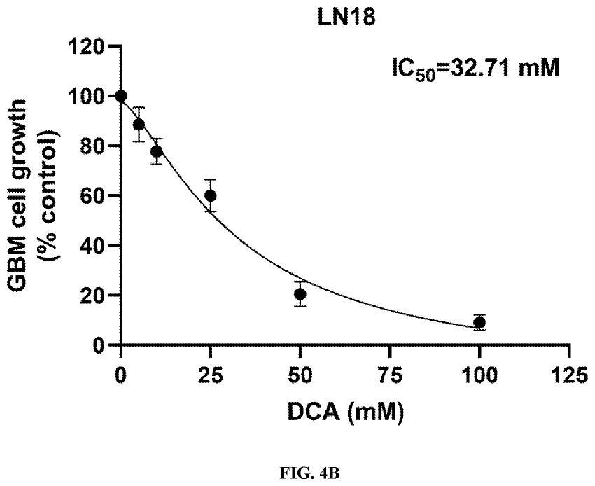

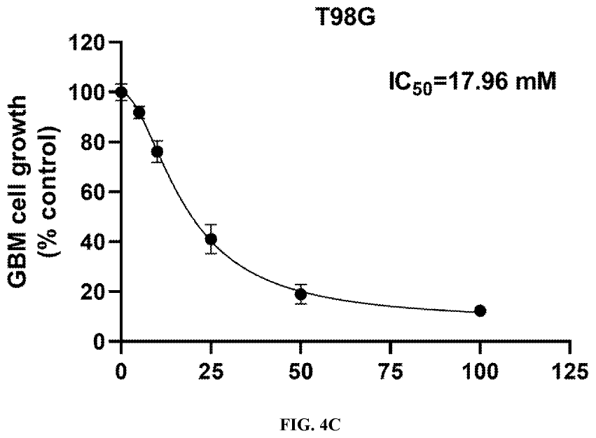

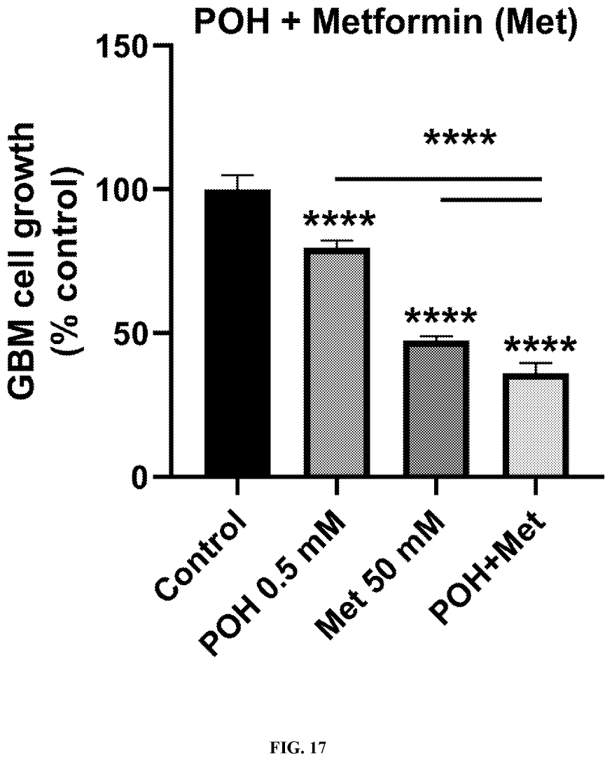

As a result of this experimental scenario, both POH and DCA displayed a direct and dose-dependent antiproliferative effect on rapidly-growing GBM cell cultures, obtaining IC₅₀ values of 1.01 (for LN229 cell line, FIG. 3A), 0.77 (for LN18 cell line, FIG. 3B) and 0.76 mM (for T98G cell line, FIG. 3C) were obtained for POH, and 33.31 (for LN229 cell line, FIG. 4A), 32.71 (for LN18 cell line, FIG. 4B) and 17.96 mM (for T98G cell line, FIG. 4C) for DCA (FIG. 4). Significant (p<0.05) cytostatic activity was observed from 0.5-0.75 mM for POH, and 12.5-25 mM for DCA concentrations onwards, depending on tumor cell line.

To confirm synergistic effects of POH and DCA cell proliferation assays on LN229 cultures were conducted using a combinational matrix design. Treatments were conducted using optimal and suboptimal concentrations of each tested compound.

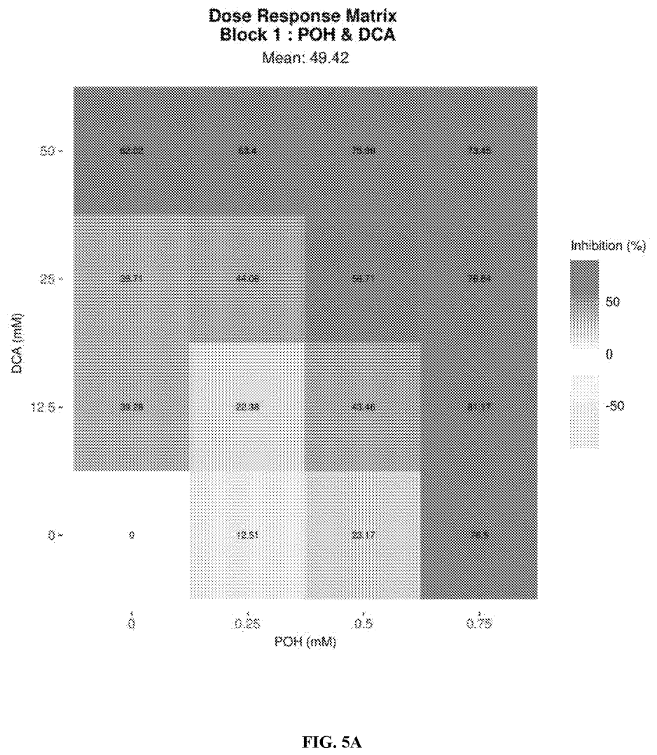

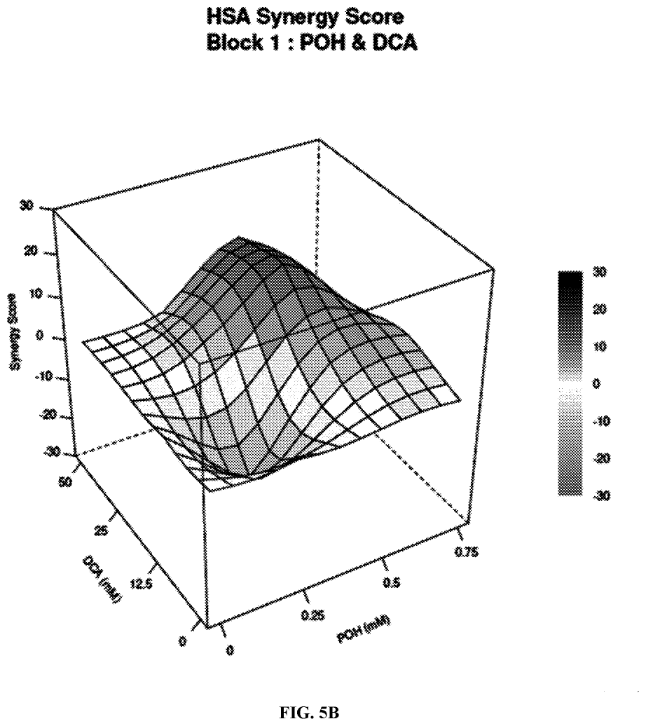

POH and DCA combination using different concentrations resulted in a marked inhibitory activity on GBM exponential growth, as evidenced in the heat map-based dose-response matrix (FIG. 5A). After integrating these results, an average synergy score of 2.88 was obtained using the HSA model (Values >0 represent synergistic interaction between drug pairs) (FIG. 5B). Furthermore, synergistic CI values <1 were obtained in 8 out of 9 POH+DCA combinations (Table 1).

TABLE 1

| Dose POH | Dose DCA | Effect | CI |
|---|---|---|---|
| 1.0E−6 | 1.0E−6 | 1.0E−6 | 1.54487 |
| 0.25 | 12.5 | 0.2238 | 1.99660 |
| 0.5 | 12.5 | 0.4346 | 0.94131 |
| 0.75 | 12.5 | 0.8117 | 0.20329 |
| 0.25 | 25.0 | 0.4406 | 0.95143 |
| 0.5 | 25.0 | 0.5671 | 0.70109 |
| 0.75 | 25.0 | 0.7684 | 0.32000 |
| 0.25 | 50.0 | 0.634 | 0.61936 |
| 0.5 | 50.0 | 0.7599 | 0.37481 |
| 0.75 | 50.0 | 0.7345 | 0.52317 |

Combinational Indexes (CI) between 0-1 represent synergy and values >1 represent antagonism (Compusyn Software, USA).

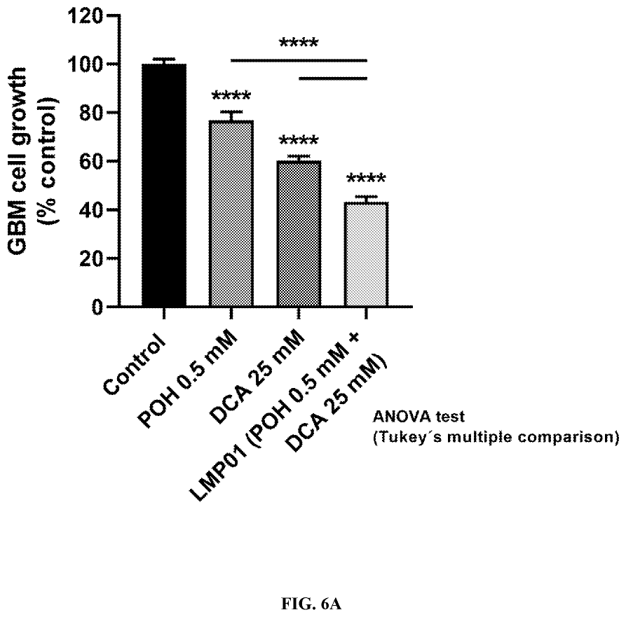

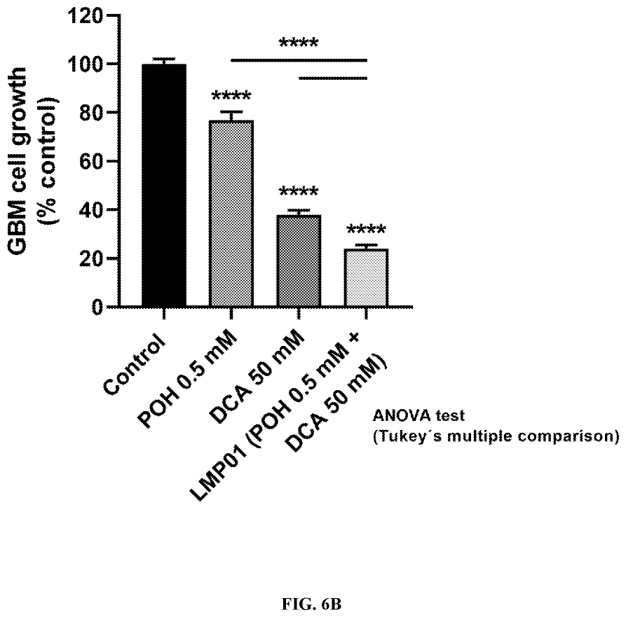

Representative drug combinations using specific concentrations within high synergistic areas (POH 0.5 mM plus DCA 25 mM, and POH 0.5 mM plus DCA 50 mM) are depicted in FIG. 6A and FIG. 6B, respectively. In these combinatorial schemes it is highly evident that dual concomitant exposure of POH in addition to DCA results in a significant anti-GBM activity, improving the therapeutic benefits of either monotherapy alone. In this regard, LMP01 potent combined antiproliferative action may stall the progression of rapidly growing recurrent GBM lesions.

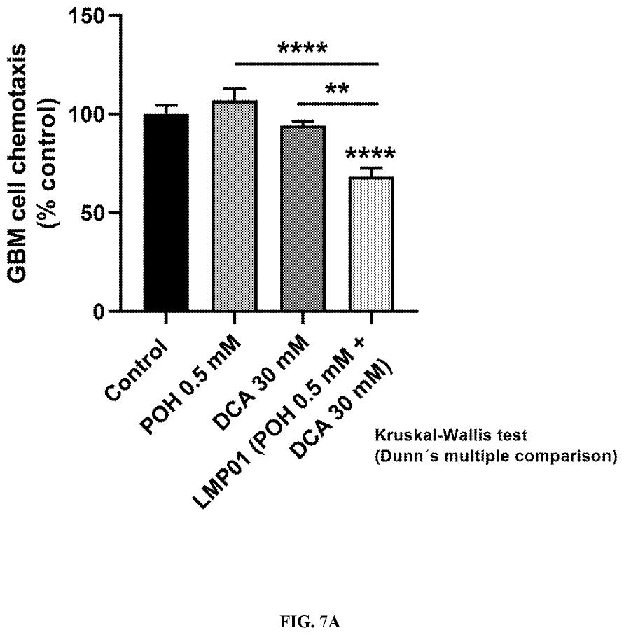

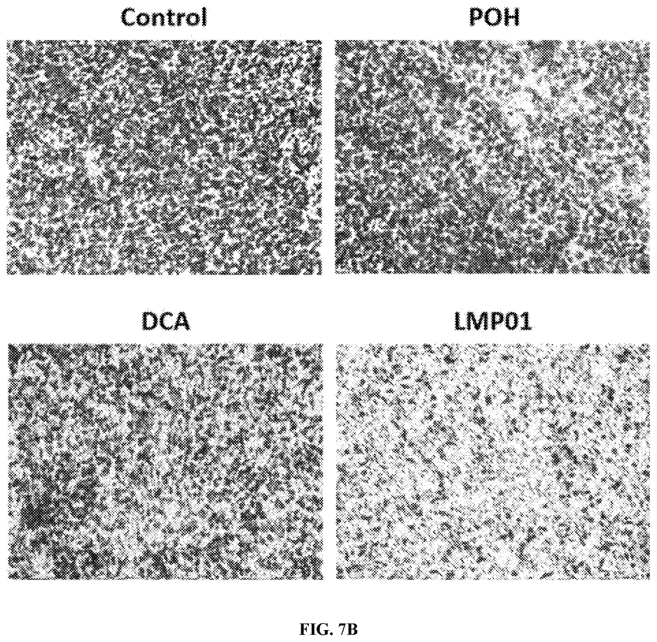

Continuing with the evaluation of the therapeutic action of LMP01, the impact of this combined therapy on the phenomenon of tumor chemotaxis of GBM cells was studied. As observed in the control group, vehicle-treated cells exhibited a highly migratory and aggressive phenotype (FIG. 7A and FIG. 7B). In this scenario, POH nor DCA as monotherapies were unable to affect GBM migration after short term exposure. On the contrary, LMP01 combination therapy (0.5 mM POH plus 30 mM DCA) displayed a significant inhibitory activity on tumor cell motility of LN229 cells, down modulating chemotactic capacity by 32% in treated cells, outperforming each monotherapy. A non-significant 6% reduction in GBM chemotaxis was observed after DCA treatment at 30 mM.

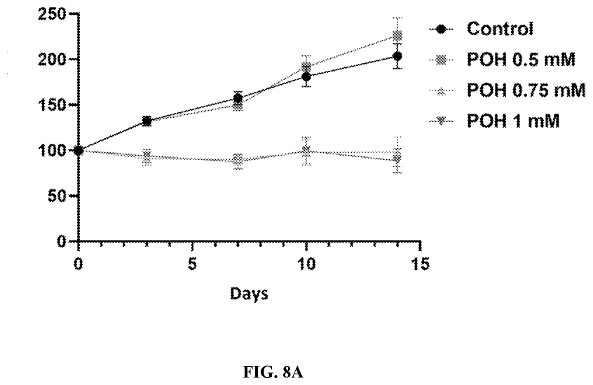

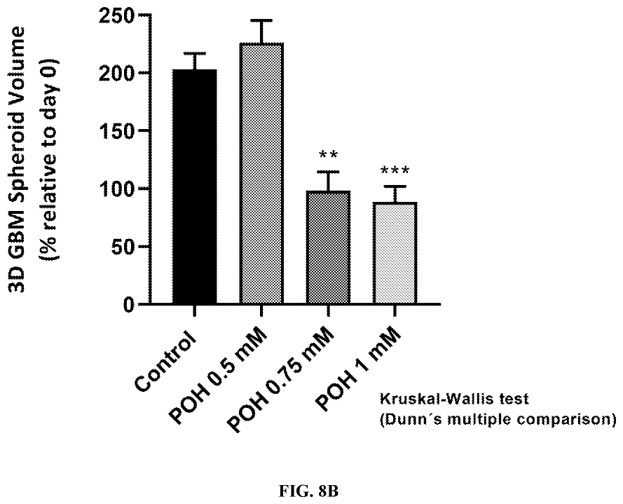

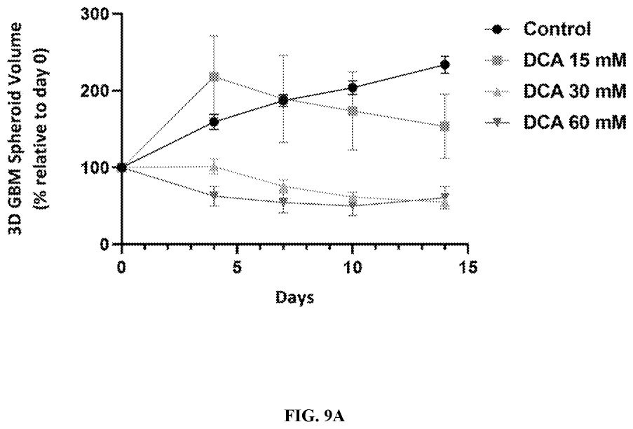

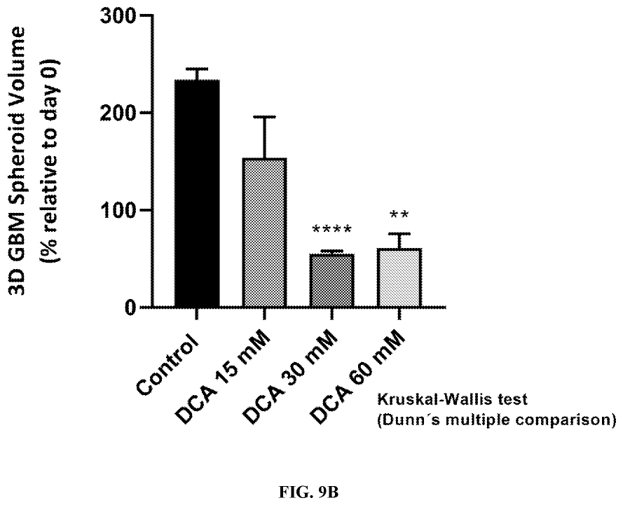

The effect of LMP01 on 3D tumor spheroid growth was also evaluated using a model that mimics more closely the in vivo tumor microenvironment compared to traditional two-dimensional cultures (Manduca N, et al., Front Immunol. 2023; 14:1175503). First, the effect of POH as monotherapy on LN229 3D cell growth was evaluated (FIG. 8A). Concentrations of 0.5, 0.75, and 1 mM POH were evaluated, revealing growth inhibition starting at 0.75 mM. The inhibitory effect reached saturation, with no enhancement upon increasing the concentration to 1 mM. In contrast, 0.5 mM POH showed no effect on 3D cell proliferation. This trend was confirmed by analyzing tumor volume at the end of the experiment (day 14), where both 0.75 mM and 1 mM POH induced a significant reduction in tumor volume compared to the control (FIG. 8B). Next, the efficacy of DCA as a monotherapy was assessed in the LN229 3D model (FIG. 9A). Tested concentrations (15, 30, and 60 mM) demonstrated that growth inhibition was reached starting at 30 mM. This observation aligned with tumor volume measurements at the endpoint (day 14), where both 30 and 60 mM DCA induced a significant reduction compared to control (FIG. 9B). Both compounds significantly impacted on 3D tumor growth, but with distinct effects: POH induced proliferative arrest (no volume increase compared to day 0), whereas DCA produced a significant volume reduction (40-45% decrease at concentrations = or > than 30 mM). These differences reflect different mechanisms of action for both drugs evaluated.

Based on the effective concentrations identified in monotherapy assays, various drug combinations were evaluated.

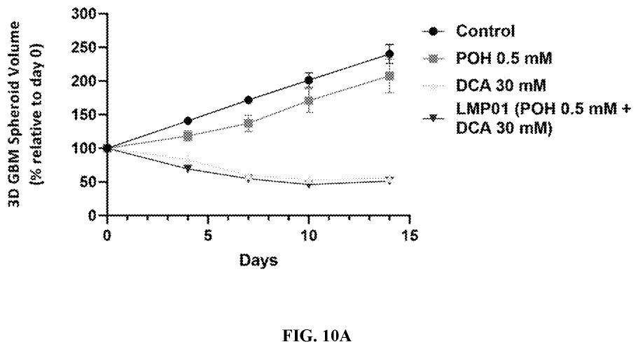

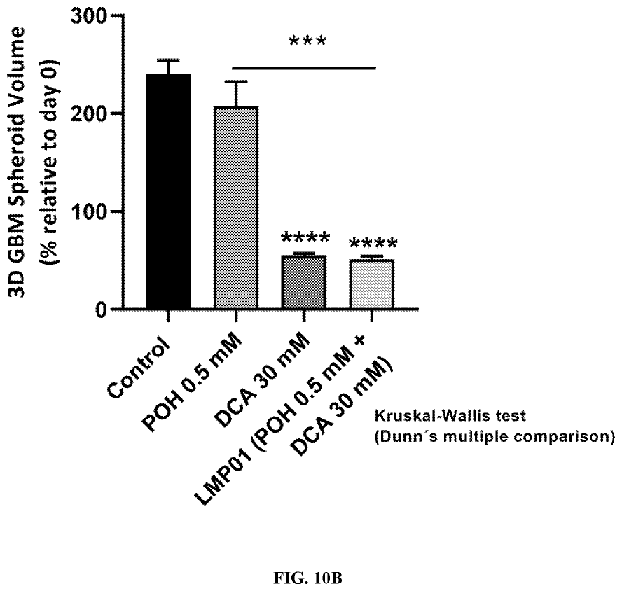

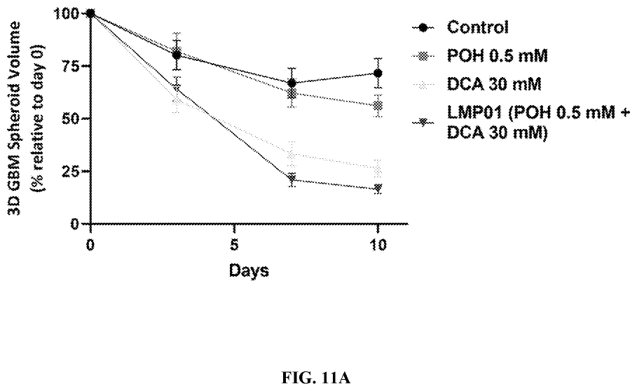

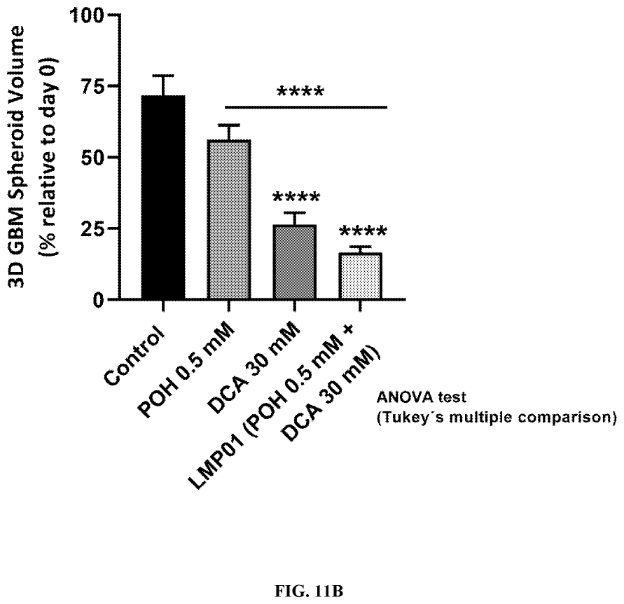

The 0.5 mM POH plus 30 mM DCA combination revealed that DCA's inhibitory effect reached saturation, significantly reducing 3D proliferation in both spheroid growth kinetics and endpoint analysis (day 14) (FIG. 10A). Under these conditions, POH did not exhibit any capacity to enhance DCA's antiproliferative effect. Independent validation using 0.5 mM POH plus 30 mM DCA in LN18 3D spheroids showed comparable results at endpoint (day 10, FIG. 10B). The combination treatment resulted in the greatest significant inhibitory effect on 3D growth compared to the control (84% growth reduction). LMP01 demonstrated superior anti-GBM activity compared with POH at the suboptimal concentration of 0.5 mM concentration (FIG. 11A and FIG. 11B).

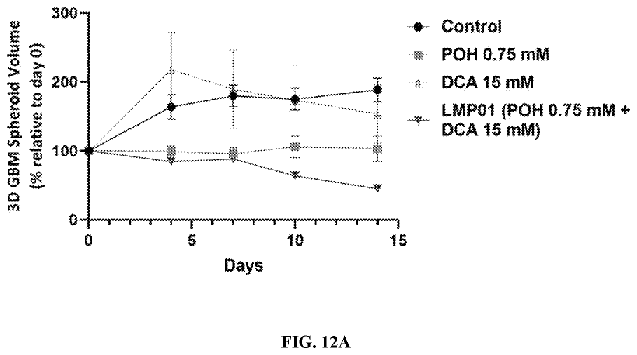

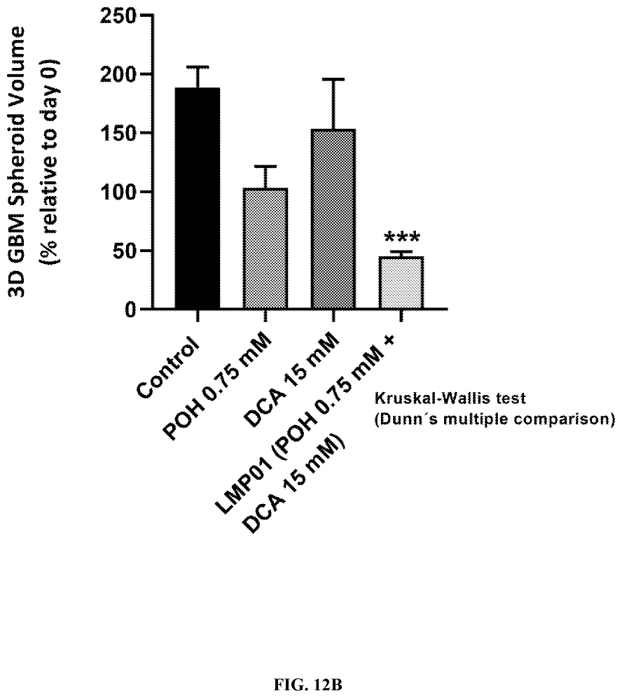

Based on these findings, a second drug combination was evaluated in the main experimental model LN229: 0.75 mM POH plus 15 mM DCA. Growth curve analysis revealed that this combination outperformed both monotherapies starting on day 7, achieving a 75% proliferation inhibition by the endpoint (day 14) compared to the control group. (FIG. 12A). Under these experimental conditions, LMP01 combining 0.75 mM POH plus 15 mM DCA, was the treatment capable of significantly impairing 3D growth on day 14 in comparison to vehicle-treated spheroids (FIG. 12B).

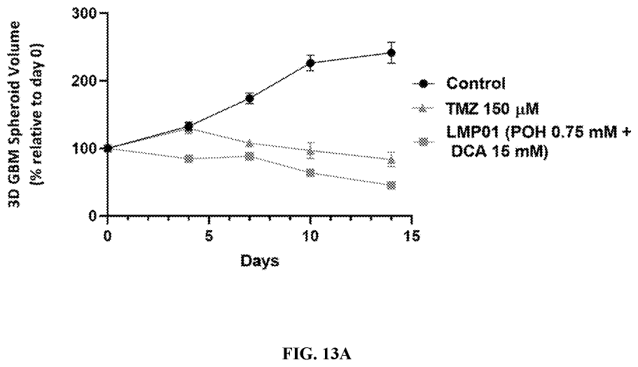

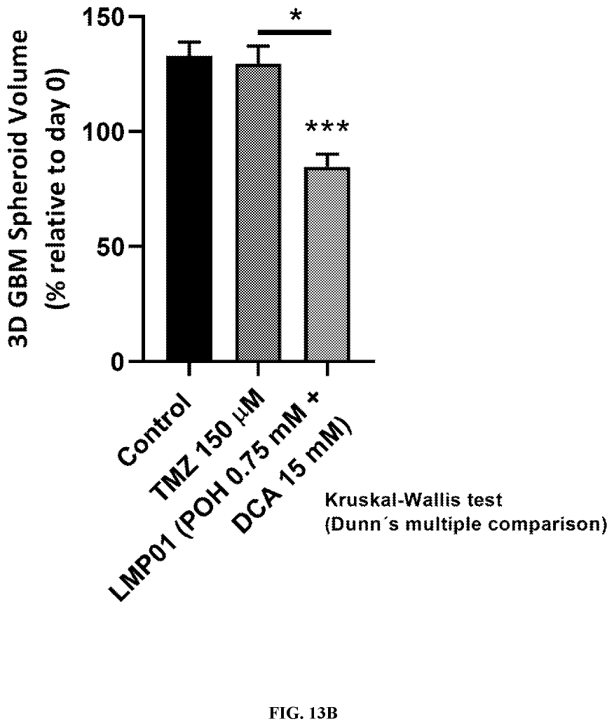

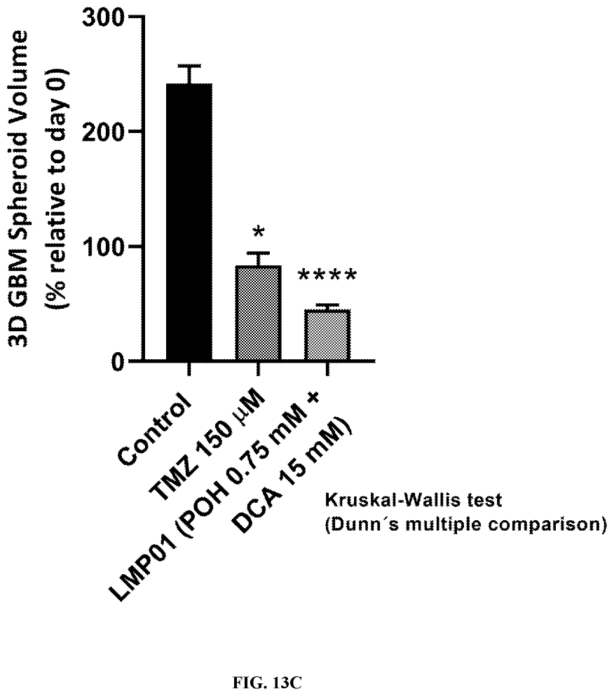

Strikingly, by endpoint at day 14, LMP01 (0.75 mM POH+15 mM DCA) therapeutic activity was comparable to that of the standard-of-care chemotherapeutic agent TMZ used at high cytotoxic concentrations (150 μM). Notably, the LMP01 combination significantly performed TMZ during the early phase of spheroid outgrowth (day 4) (FIG. 13A). Collectively, 2D and 3D findings demonstrate that the LMP01-based therapy exhibits superior antitumor efficacy in GBM models compared to either agent alone or standard TMZ treatment at high concentrations. (FIG. 13B, at day 4, and FIG. 13C, at day 14).

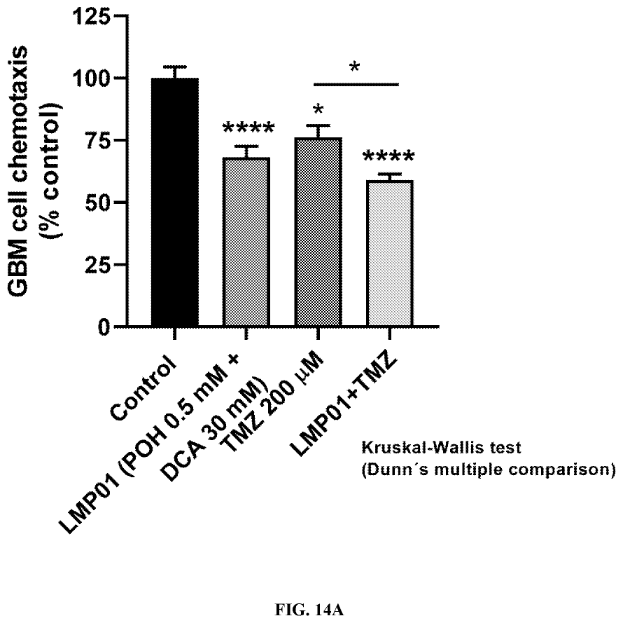

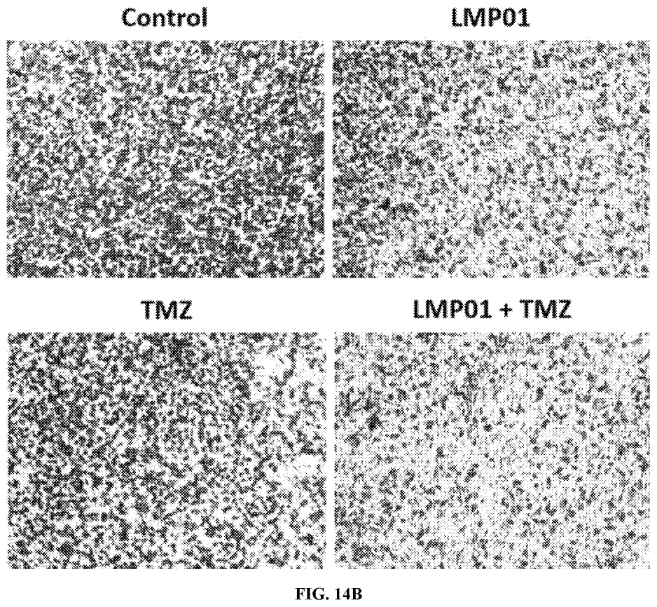

To assess potential therapeutic benefits after adding LMP01 to TMZ the combinatorial impact on GBM cell migratory capacity was first evaluated. As shown in FIG. 14A, the concomitant treatment using LMP01 in addition to high TMZ concentrations resulted in a cooperative inhibitory effect on GBM chemotaxis (41% inhibition), significantly enhancing therapeutic benefits of reference chemotherapy alone (23.8% inhibition). Representative photos from different experimental groups depicting migrated GBM cells are shown in FIG. 14B.

In summary, LMP01 has been evaluated in various experimental models of human GBM characterized by high proliferative capacity, invasiveness, and differing MGMT expression status. As previously stated, high proliferation rates in GBM are biologically significant as they correlate directly with tumor aggressiveness, growth dynamics, recurrence, and patient prognosis. Treatment with LMP01 induced a potent cytostatic effect in exponentially growing, extremely aggressive GBM cultures. Using multiple high-density proliferation assays, a 72-hour exposure to LMP01 demonstrated dose-dependent antiproliferative activity when the two rationally selected small molecules were combined at micromolar to low millimolar concentrations.

The combination therapy outperformed each monotherapy, with synergy confirmed at specific concentration ranges. Moreover, GBM is characterized by its highly infiltrative and migratory phenotype, a major contributor to its lethality and poor prognosis. Notably, LMP01 impaired GBM cell motility, as demonstrated by transwell migration assays. After only 16 hours of treatment, tumor cell chemotaxis was significantly inhibited compared to vehicle controls and individual treatments.

GBM cells surviving surgical resection and adjuvant therapies can form highly proliferating residual colonies, leading to recurrence and multifocal disease. To address this, LMP01's effect on 3D GBM growth was assessed using a long-term spheroid progression assay. After two weeks of treatment, LMP01 combination therapy significantly inhibited 3D tumor growth, reducing GBM spheroid volume by up to 75% in comparison to control. This therapeutic activity was comparable to that of the standard-of-care chemotherapeutic agent TMZ used at high cytotoxic concentrations.

### Example 3

### In Vivo Assays with POH-DCA Combinations

In this next example, the therapeutic efficacy of LMP01 was evaluated in an orthotopic glioblastoma model using LN229 cells implantation. For this purpose, LN229 human glioblastoma cells were implanted in the brain of immunocompromised mice (NLAE: NIH(S)-Fox1nu “nude” mice) and then treated with a daily dose of LMP01 using two different administration routes, compared to vehicle-treated control group. The following materials and protocols were used:

LN229 (ATCC CRL-2611) is an extremely aggressive human GBM cell model, widely used in biomedical research. It was isolated in 1979 from the right frontal parieto-occipital cortex of a white, 60-year-old, female patient with GBM. It has an epithelial-like morphology, and it is tumorigenic in nude mice. It bears a mutation in p53 (TP53) and possible homozygous deletions in the p16 and p14ARF tumor suppressor genes. LN229 cells were grown using Dulbecco's modified Eagle's medium (DMEM) culture medium (Gibco, USA) supplied with 10% fetal bovine serum (FBS) (Natocor, Córdoba, AR), 80 μg/mL gentamicin, and 2 mM glutamine, in monolayer culture, at 37 °C in a humidified atmosphere of 5% CO₂. Cells were harvested using a trypsin/EDTA solution (Thermo Fisher Scientific Inc., USA) diluted in PBS and routinely evaluated for mycoplasma.

The LN229 cells where then implanted in the brain of male nude mice, used to perform the in vivo experiment. All animals were 6-8 weeks old and weighed approximately 20 g. All the animals in these studies were produced in the animal facility of the Veterinary Faculty of La Plata University (Buenos Aires, AR). The animals were housed in an animal isolator and animal manipulation was performed in laminar-air-flow units. All animals were given ad libitum access to food and water.

All animals were anesthetized using i.p administration of ketamine/xylazine (80:20). Sedation was monitored using a gentle toe pinch withdraw reflex. The animal head was positioned and secured in a stereotactic frame (Stoelting, USA). The eyes were moistened with eye drop solution to prevent damage to the animal's eye. The scalp was disinfected with povidone/iodine and an anterior/posterior incision was made with a sterile scalpel extending from the lambda to just in-between the eyes of the animal. The exposed skull surface was then cleaned using a cotton swab soaked in a 3% hydrogen peroxide solution. A burr hole was made 0.1 mm anterior to bregma and 2.3 mm to the right of the midline. A 33G needle (10 μL Hamilton syringe) was inserted to a depth of 3 mm and withdrawn 0.4 mm to a depth of 2.6 mm. 2×10⁵ human glioblastoma LN229 cells were delivered in 2 μL at a rate of 0.5 L/min. After injection was completed, the needle was left in the brain for approximately 2 min and then slowly withdrawn over a period of 3-4 min. Then the ear bars were loosened, and the animal was removed from the stereotactic apparatus. To seal the wound, the edges of the incision were re-approximated, cleaned with sterile cotton swabs and veterinarian tissue glue was applied. Finally, the mouse was placed at 37 °C until the animal recovered consciousness. Once the mouse was alert and responsive, it was transferred to its original cage.

Tramadol and ampicillin were administered for the next 5 days after the surgery. POH (MW 152.23 g/mol, Sigma Aldrich, USA) was added to saline and then vigorously vortexed for 5 seconds before every administration. DCA (MW 150.92 g/mol, Curaltus Ltd., Vilnius, LT) was dissolved in saline and diluted in sterile water, which was provided as drinking water ad libitum. The DCA-containing water was replaced three times per week.

The combination LMP01 i.n. was prepared by adding POH to a DCA solution in saline solution. Prior to each administration, the mixture was vigorously vortexed for 5 seconds.

All groups received a daily administration (24 h±2h) of vehicle or treatment. Control group received intranasal (i.n.) administration of 12.5 μl of saline solution. LMP01 (oral+i.n.) received POH 0.3125 mg/day was administered via i.n. on average, each animal consumed ~1.5 mg/day of DCA through the drinking water. LMP01 (i.n.) received i.n. administration of POH 0.3125 mg/day and DCA 1 mg/day combined. 7 days post-surgery the animals were separated randomly into distinct groups of 4 to 5 mice each.

The treatment consisted of daily administration of the before-mentioned drugs. All mice were monitored daily, weighed twice a week and consumption of water was registered 3 times/week.

Animals were euthanized if they met any of the following criteria: weight loss (>20%), physical deterioration, cachexia, abnormal mobility, lethargy, ataxia, and/or a hunched posture. Following this procedure, animals were necropsied, and brains were formalin-fixed, paraffin-embedded, and sectioned for histological analysis. Tissue sections were mounted on glass slides and stained with hematoxylin and eosin (H&E) to assess tumor morphology, invasion patterns, and pathological features.

Statistical analyses and graphs were performed using GraphPad Prism (USA) and Statas Calculator (source: https://astatsa.com) with significance thresholds denoted as follows: **p<0.05 by χ² test for proportions; #0.05≤p<0.10 by Gehan-Wilcoxon test (Peto & Peto modification) for survival curves; *p<0.05 and &0.05≤p<0.10 by one-tailed t-tests for directional hypotheses.

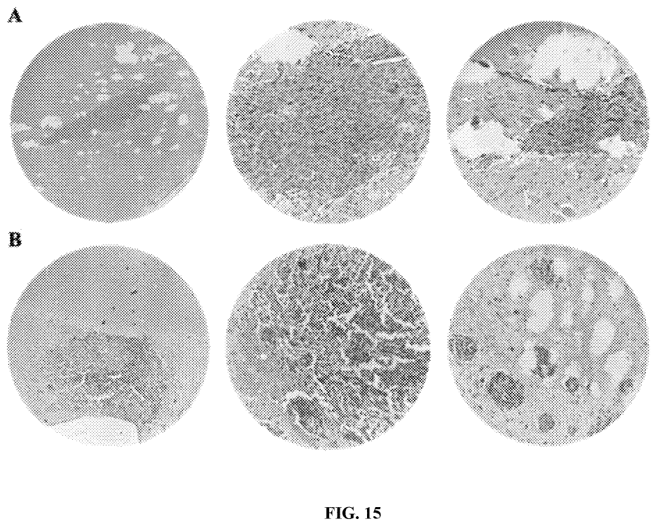

Histopathological confirmation at day 7 post-implantation (treatment day 0) revealed small, well demarcated tumors at the injection site, establishing consistent engraftment (FIG. 15A). Tumor progression was demonstrated by evaluating the first control animal meeting euthanasia criteria. This animal exhibited classical GBM histopathology-including microvascular proliferation, and frequent mitotic activity with distinct prophase and metaphase figures (FIG. 15B).

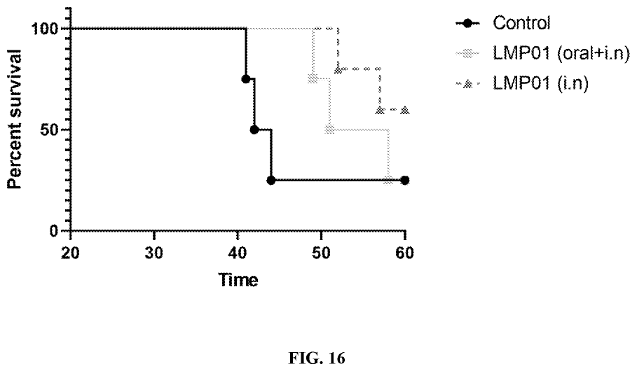

At a 60-day interim analysis, LMP01 demonstrated significant survival extension (Mean survival: 57 days for LMP01 in and 54 days for LMP01 oral+i.n.) versus vehicle controls (Mean survival 46 days) (Table 2, FIG. 16). Notably, the intranasal combination regimen showed superior efficacy over oral-intranasal co administration.

TABLE 2

| | | | 50% | | Mean | |
|---|---|---|---|---|---|---|
| | Survival at | P versus | survival | P versus | survival at | P versus |
| Group | day 30 | control | reached | control | day 60 | control |
| Control | 25% (1/4) | — | 43 days | — | 46 days | — |
| vehicle | | | | | | |
| LMP01 | 100% (5/5) | P = 0.017** | >60 days | P = 0.076^{#} | 57 days | P = 0.018 |
| i.n. | | | | | | |
| LMP01 | 75% (3/4) | NS | 52 days | NS | 54 days | P = 0.093^{&} |
| oral + i.n. | | | | | | |

**Significant (Chi square test);
^{#}Not quite significant (Gehan-Wilcoxon test with Peto & Peto modification);
*Significant (one-tailed T test);
^{&}Not quite significant (one-tailed T test)

### Example 4

### In Vitro Assays with Glycolysis Inhibitors/Modulators and Prenyltransferase Inhibitors Combinations

Various assays were conducted with a panel of compounds that are functionally analogous to DCA and POH (i.e., in terms of mechanisms of action and molecular targets) for evaluating their potential anti-GBM synergistic effects. The objectives were analyzing (a) the cooperative therapeutic profiles and (b) complementary antitumor cellular and molecular mechanisms of these compounds when administered concomitantly. The following materials were used:

### POH or POH Analogs

POH (MW 152.23 g/mol, Sigma Aldrich, USA). Considering the maximum non-toxic dose for cells (1% DMSO), the compound was diluted using dimethyl sulfoxide (DMSO) to reach final working concentrations.

Lonafarnib (MW 638.82 g/mol, Millipore Sigma, USA). Considering the maximum non-toxic dose for cells (1% DMSO), the compound was diluted using dimethyl sulfoxide (DMSO) to reach final working concentrations.

6-Gingerol (MW 294.4 g/mol, Sigma-Aldrich). Considering the maximum non-toxic dose for cells (1% DMSO), the compound was diluted using dimethyl sulfoxide (DMSO) to reach final working concentrations.

### DCA or DCA Analogs

DCA (MW 150.92 g/mol, Curaltus Ltd, Vilnius, LT). The drug was first solubilized using sterile water to generate concentrated stocks and subsequent dilutions using sterile water were conducted to generate final working concentrations.

2-Deoxy-D-glucose (2DDG, MW 164.16 g/mol, Millipore Sigma, USA). The drug was first solubilized using sterile water to generate concentrated stocks and subsequent dilutions using sterile water were carried out to generate final working concentrations.

Metformin HCl (MW 165.6 g/mol, Sigma-Aldrich, USA). The drug was first solubilized using sterile water to generate concentrated stocks and subsequent dilutions using sterile water were carried out to generate final working concentrations.

### Cell Lines

LN229 (ATCC CRL-2611) is a highly aggressive human GBM cell model, widely used in biomedical research. It was isolated in 1979 from the right frontal parieto-occipital cortex of a white, 60-year-old, female patient with GBM. It has an epithelial-like morphology, and it is tumorigenic in nude mice. It bears a mutation in p53 (TP53) and possible homozygous deletions in the p16 and p14ARF tumor suppressor genes.

LN229 cells were grown using Dulbecco's modified Eagle's medium (DMEM) culture medium (Gibco, USA). Cells were supplied with 10% fetal bovine serum (FBS) (Natocor, Córdoba, AR), 80 μg/mL gentamicin, and 2 mM glutamine, in monolayer culture, at 37 °C in a humidified atmosphere of 5% CO₂. Cells were harvested using a trypsin/EDTA solution (Thermo Fisher Scientific Inc., USA) diluted in PBS and routinely assessed for mycoplasma.

### 1. GBM Log-Phase Proliferation

A high-density log-phase proliferation assay was used to evaluate GBM cellular growth. Briefly, LN229 cells were plated in 96 multi-well plate format at a density of 2×10\hat{ }3 cells per 100 μL of complete DMEM medium. After 24 h, cells were exposed to POH- or DCA-analog compounds. 72 h later exponentially growing cell cultures were fixed for 10 min with methanol and then stained with 0.5% crystal violet solution for another 10 min.

Following a washing step, fixed cultures were dried overnight. Quantification for the crystal violet method was performed by absorbance measurement (2=595 nm) after adding an ethanol/acetic acid 3:1 solution. Values are presented as a percentage of control. The tests were performed by duplicate (independent operators), employing 6-12 wells per experimental group per independent replicate.

2. Synergy Assessment with Selected Concentrations

To assess any possible synergistic effect by combining both POH- and DCA-analogs, different high-density cell proliferation assays were carried out in the exponential phase for 72 h. The potential cooperative effects between drug pairs were evaluated using a combinational matrix design.

For this purpose, LN229 cells were seeded at a density of 2×10\hat{ }3 cells per well, allowed to attach overnight, and then treated using optimal (IC₅₀) and suboptimal (IC₁₀ and IC₂₅) concentrations of the drugs.

For subsequent comparative statistical analysis between experimental groups, outlier identification, analysis of combination indexes, dose-response curve shifts, isobolograms, dose reduction indexes, and synergy scores, the statistical packages PRISM GraphPad, Compusyn, and SynergyFinder were used.

### 3. Statistics

In vitro assay determinations were conducted from at least 2 independent experiments. Statistical analyses were mainly performed using GraphPad Prism 8.0V software, (GraphPad Prism Software, USA). Distribution of data and descriptive statistics were firstly assessed. Outliers were detected and removed (Prism calculator).

For comparisons among 3 or more experimental groups, ANOVA or its non-parametric equivalent, the Kruskal-Wallis test, were utilized, depending on the parametric or non-parametric distribution of values, respectively. Subsequently, tests such as Dunnett's test, Tukey's test, comparison of 95% confidence intervals (CI) for the mean (ANOVA), or Dunn's test (Kruskal-Wallis) were employed.

Synergy was confirmed when CI<1 or integrated HSA scores>0. For the calculation of the half inhibitory concentration (IC₅₀), nonlinear regressions were applied, based on optimal curve-fitting. Differences were considered statistically significant at a level of p<0.05; *p<0.05; **p<0.01; ***p<0.001; ****p<0.0001.

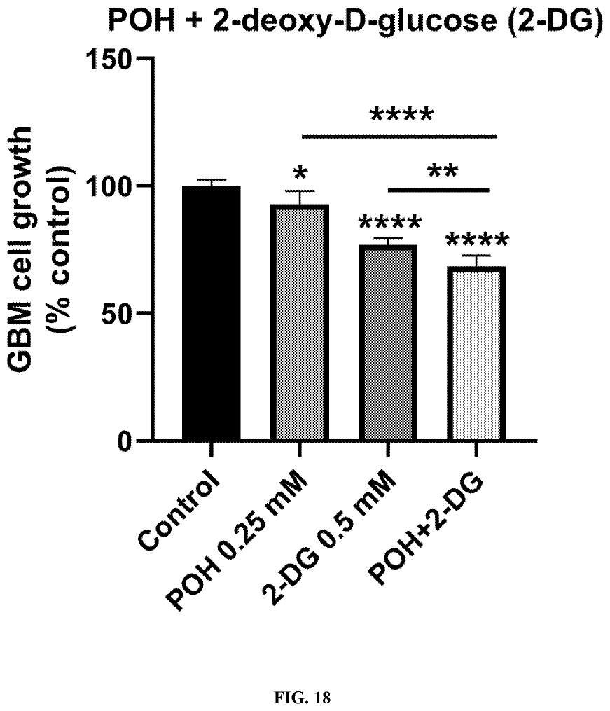

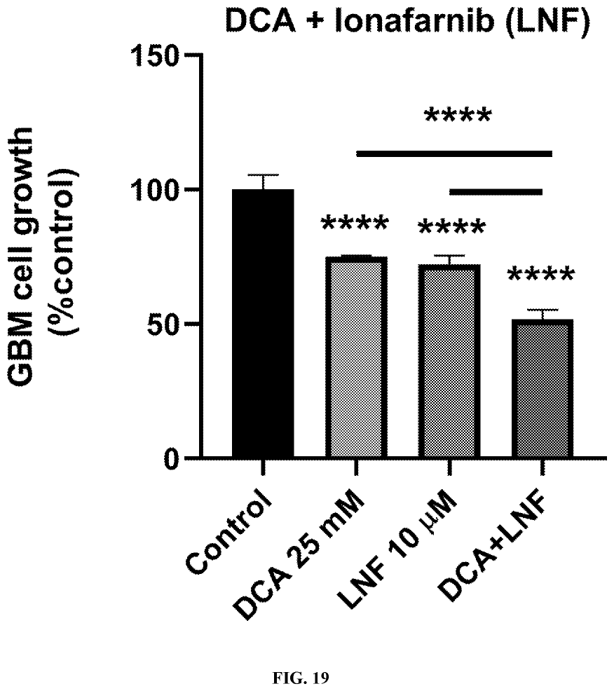

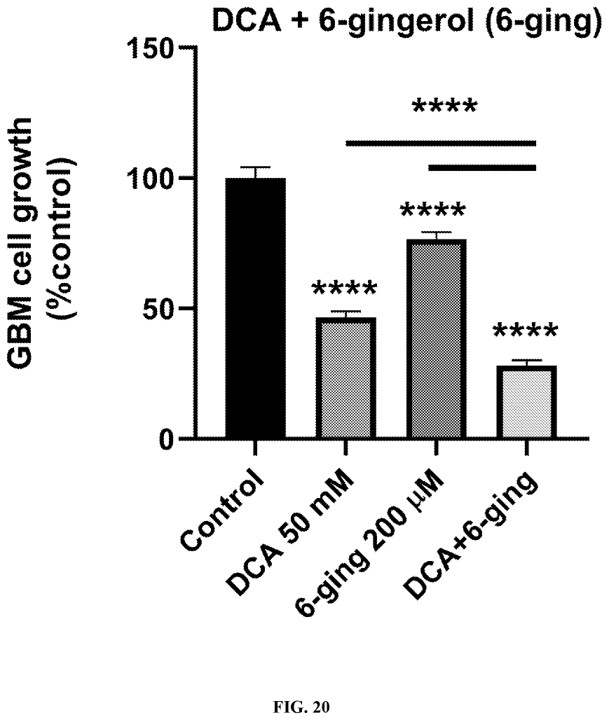

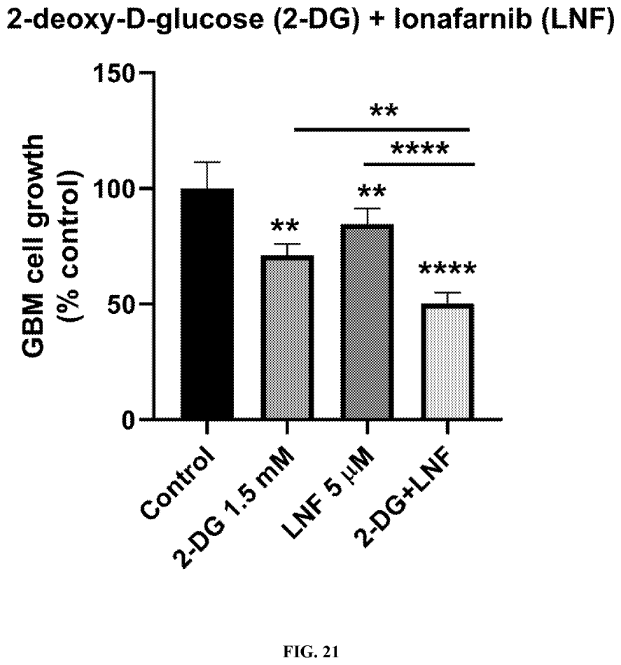

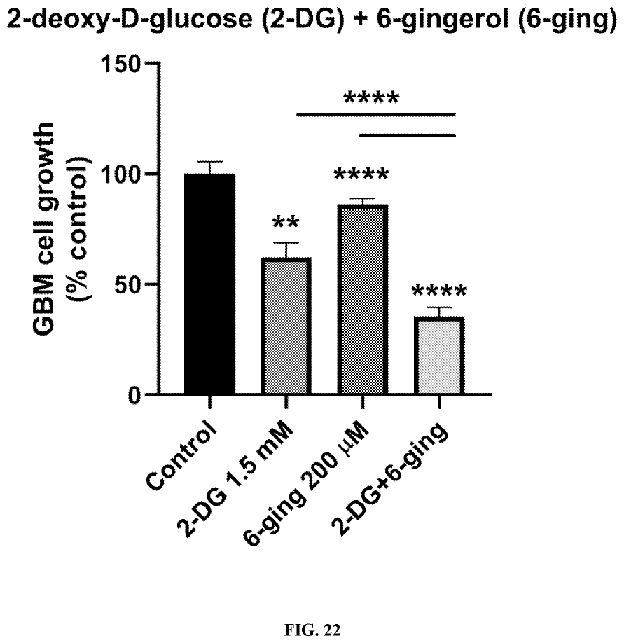

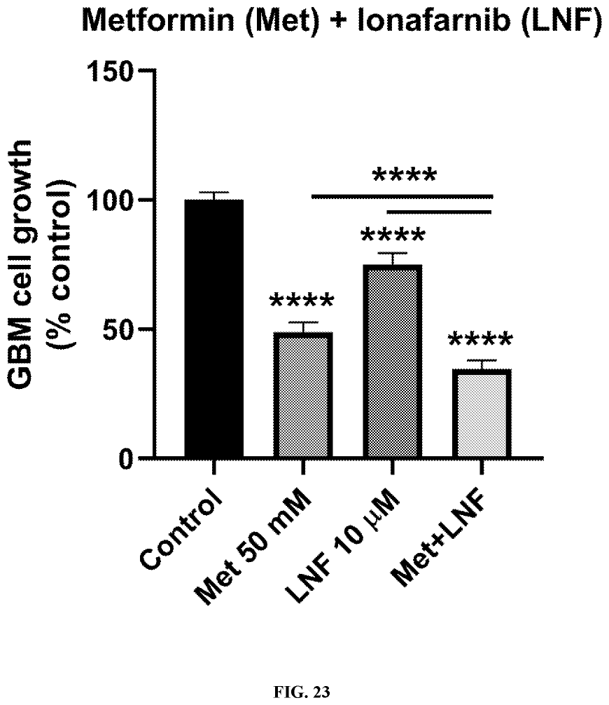

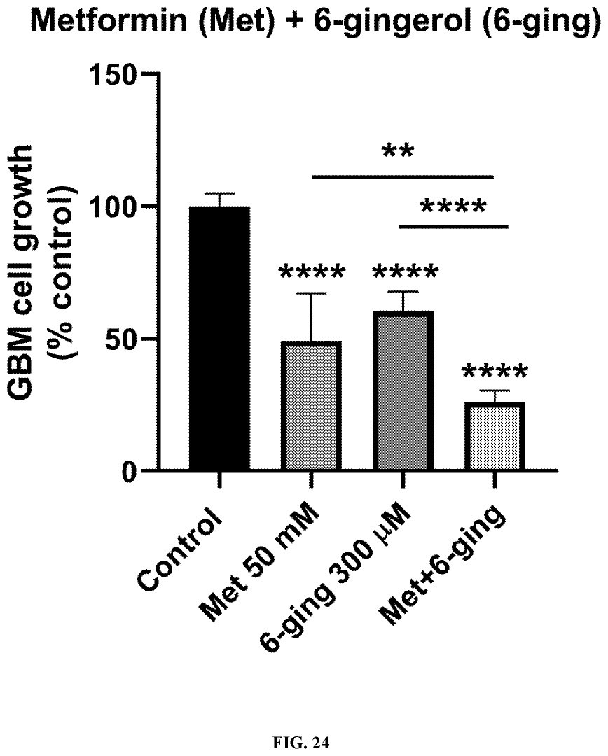

The following particular combinations glycolysis inhibitors/modulators and prenyltransferase inhibitors were tested:
(1) POH plus metformin HCL (FIG. 17, Table 3),
(2) POH plus 2DDG (FIG. 18, Table 4),
(3) DCA plus lonafarnib (FIG. 19, Table 5),
(4) DCA plus 6-gingerol (FIG. 20, Table 6),
(5) 2DDG plus lonafarnib (FIG. 21, Table 7),
(6) 2DDG plus 6-gingerol (FIG. 22, Table 8),
(7) metformin HCl plus lonafarnib (FIG. 23, Table 9), and
(8) metformin HCl plus 6-gingerol (FIG. 24, Table 10).

The results showed high synergy (scores >>>0) and combinational indexes <1 for all the combinations of active agents tested, in a dose dependent manner.

TABLE 3

| Dose POH | Dose metformin HCl | Effect | CI |
|---|---|---|---|
| 0.25 | 10.0 | 0.193 | 1.69869 |
| 0.25 | 20.0 | 0.3185 | 1.20905 |
| 0.25 | 50.0 | 0.5061 | 0.95340 |
| 0.5 | 10.0 | 0.3554 | 0.95533 |
| 0.5 | 20.0 | 0.4395 | 0.86917 |
| 0.5 | 50.0 | 0.639 | 0.58420 |
| 0.75 | 10.0 | 0.4765 | 0.73293 |
| 0.75 | 20.0 | 0.5359 | 0.70329 |
| 0.75 | 50.0 | 0.7091 | 0.46766 |

Combinational Indexes (CI) between 0-1 represent synergy and values >1 represent antagonism (Compusyn Software, USA).

TABLE 4

| Dose POH | Dose 2DDG | Effect | CI |
|---|---|---|---|
| 0.25 | 0.5 | 0.3703 | 0.58704 |
| 0.25 | 1.5 | 0.5546 | 0.63088 |
| 0.25 | 5.0 | 0.4993 | 2.40279 |
| 0.5 | 0.5 | 0.304 | 1.05759 |
| 0.5 | 1.5 | 0.5249 | 0.81224 |
| 0.5 | 5.0 | 0.6245 | 1.46780 |
| 0.75 | 0.5 | 0.4415 | 0.70578 |
| 0.75 | 1.5 | 0.6455 | 0.53854 |
| 0.75 | 5.0 | 0.6685 | 1.25076 |

Combinational Indexes (CI) between 0-1 represent synergy and values >1 represent antagonism (Compusyn Software, USA).

TABLE 5

| Dose DCA | Dose lonafarnib | Effect | CI |
|---|---|---|---|
| 12.5 | 5.0 | 0.3211 | 0.72022 |
| 25.0 | 5.0 | 0.4265 | 0.58608 |
| 50.0 | 5.0 | 0.5982 | 0.38965 |
| 12.5 | 10.0 | 0.3353 | 1.04064 |
| 25.0 | 10.0 | 0.482 | 0.62140 |
| 50.0 | 10.0 | 0.574 | 0.55628 |
| 12.5 | 20.0 | 0.4867 | 0.82821 |
| 25.0 | 20.0 | 0.5854 | 0.58611 |
| 50.0 | 20.0 | 0.5227 | 0.87385 |

Combinational Indexes (CI) between 0-1 represent synergy and values >1 represent antagonism (Compusyn Software, USA).

TABLE 6

| Dose DCA | Dose 6-gingerol | Effect | CI |
|---|---|---|---|
| 12.5 | 200.0 | 0.5626 | 0.31862 |
| 25.0 | 200.0 | 0.5609 | 0.37539 |
| 50.0 | 200.0 | 0.7183 | 0.18949 |
| 12.5 | 300.0 | 0.5162 | 0.58397 |
| 25.0 | 300.0 | 0.6013 | 0.40560 |
| 50.0 | 300.0 | 0.7265 | 0.22779 |
| 12.5 | 400.0 | 0.5936 | 0.48996 |
| 25.0 | 400.0 | 0.6259 | 0.37788 |
| 50.0 | 400.0 | 0.7615 | 0.21493 |

Combinational Indexes (CI) between 0-1 represent synergy and values >1 represent antagonism (Compusyn Software, USA).

TABLE 7

| Dose 2DDG | Dose lonafarnib | Effect | CI |
|---|---|---|---|
| 0.5 | 5.0 | 0.2282 | 1.03709 |
| 1.5 | 5.0 | 04975 | 0.49954 |
| 5.0 | 5.0 | 0.5959 | 0.86503 |
| 0.5 | 10.0 | 0.3806 | 0.69790 |
| 1.5 | 10.0 | 0.4525 | 0.78538 |
| 5.0 | 10.0 | 0.6487 | 0.74362 |
| 0.5 | 20.0 | 0.5623 | 0.50300 |
| 1.5 | 20.0 | 0.6233 | 0.51110 |
| 5.0 | 20.0 | 0.6147 | 1.03284 |

Combinational Indexes (CI) between 0-1 represent synergy and values >1 represent antagonism (Compusyn Software, USA).

TABLE 8

| Dose 2DDG | Dose 6-gingerol | Effect | CI |
|---|---|---|---|
| 0.5 | 200.0 | 0.1728 | 3.51536 |
| 1.5 | 200.0 | 0.645 | 0.34154 |
| 5.0 | 200.0 | 0.6811 | 0.77184 |
| 0.5 | 300.0 | 0.5125 | 0.47278 |
| 1.5 | 300.0 | 0.6271 | 0.42663 |
| 5.0 | 300.0 | 0.6839 | 0.79587 |
| 0.5 | 400.0 | 0.6907 | 0.20015 |
| 1.5 | 400.0 | 0.7208 | 0.27808 |
| 5.0 | 400.0 | 0.7216 | 0.67567 |

Combinational Indexes (CI) between 0-1 represent synergy and values >1 represent antagonism (Compusyn Software, USA).

TABLE 9

| Dose metformin HCl | Dose lonafarnib | Effect | CI |
|---|---|---|---|
| 10.0 | 5.0 | 0.3232 | 0.60601 |
| 20.0 | 5.0 | 0.3686 | 0.67322 |
| 50.0 | 5.0 | 0.563 | 0.46805 |
| 10.0 | 10.0 | 0.3479 | 0.82933 |
| 20.0 | 10.0 | 0.3752 | 0.91069 |
| 50.0 | 10.0 | 0.6545 | 0.35113 |
| 10.0 | 20.0 | 0.4377 | 0.89966 |
| 20.0 | 20.0 | 0.4377 | 1.03904 |
| 50.0 | 20.0 | 0.7224 | 0.31837 |

Combinational Indexes (CI) between 0-1 represent synergy and values >1 represent antagonism (Compusyn Software, USA).

TABLE 10

| Dose metformin HCl | Dose 6-gingerol | Effect | CI |
|---|---|---|---|
| 10.0 | 200.0 | 0.2808 | 1.86368 |
| 20.0 | 200.0 | 0.3686 | 1.48629 |
| 50.0 | 200.0 | 0.6355 | 0.66339 |
| 10.0 | 300.0 | 0.5789 | 0.45022 |
| 20.0 | 300.0 | 0.4823 | 0.98462 |
| 50.0 | 300.0 | 0.7386 | 0.39669 |
| 10.0 | 400.0 | 0.6406 | 0.38970 |
| 20.0 | 400.0 | 0.6084 | 0.58811 |
| 50.0 | 400.0 | 0.7289 | 0.46362 |

Combinational Indexes (CI) between 0-1 represent synergy and values >1 represent antagonism (Compusyn Software, USA).

In conclusion, in this interim analysis the evaluation of LMP01 in an orthotopic glioblastoma model demonstrated antitumor efficacy, significantly extending survival compared to vehicle controls. Importantly, the intranasal administration route exhibited superior efficacy over the oral-intranasal combination, underscoring the impact of delivery optimization on treatment success. These results not only confirm LMP01's antitumor potency but also highlight the critical role of administration route optimization in maximizing therapeutic outcomes.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

Thus, those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

### REFERENCES

Alves A, et al., Role of glioblastoma stem cells in cancer therapeutic resistance: a perspective on antineoplastic agents from natural sources and chemical derivatives. Stem Cell Res Ther. 2021 Mar. 24; 12 (1): 206.
Babaloui S, et al., Radiosensitization of glioma cells by temozolomide (TMZ): a colony formation assay. J Biomed Phys Eng. 2022 Feb. 1; 12 (1): 43-50.
Berte N, et al., Artesunate enhances the therapeutic response of glioma cells to temozolomide by inhibition of homologous recombination and senescence. Oncotarget. 2016 Oct. 11; 7 (41): 67235-67250.
Birzu C, et al., Recurrent glioblastoma: from molecular landscape to new treatment perspectives. Cancers (Basel). 2020 Dec. 26; 13 (1): 47.
Cardama G, et al., Preclinical efficacy and toxicology evaluation of RAC1 inhibitor 1A-116 in human glioblastoma models. Cancers (Basel). 2022 Sep. 30; 14 (19): 4810.
Celiku O, et al., Computational modeling demonstrates that glioblastoma cells can survive spatial environmental challenges through exploratory adaptation. Nat Commun. 2019 Dec. 13; 10 (1): 5704.
Chamberlain M. Temozolomide: therapeutic limitations in the treatment of adult high-grade gliomas. Expert Rev Neurother. 2010 October; 10 (10): 1537-44.
Da Fonseca C, et al., Recent advances in the molecular genetics of malignant gliomas disclose targets for antitumor agent perillyl alcohol. Surg Neurol. 2006; 65 Suppl 1: S1: 2-1:8; discussion S1: 8-1:9.
Da Fonseca C et al., Preliminary results from a phase I/II study of perillyl alcohol intranasal administration in adults with recurrent malignant gliomas. Surg Neurol. 2008; 70:259-67.
Da Fonseca C et al., Correlation of tumor topography and peritumoral edema of recurrent malignant gliomas with therapeutic response to intranasal administration of perillyl alcohol. Invest New Drugs. 2009 December; 27 (6): 557-64.
Da Fonseca C et al., Efficacy of monoterpene perillyl alcohol upon survival rate of patients with recurrent glioblastoma. J Cancer Res Clin Oncol. 2011 February; 137 (2): 287-93. doi: 10.1007/s00432-010-0873-0. Epub 2010 Apr. 18. PMID: 20401670.
Fedorchuk A et al., Effectiveness of sodium dichloroacetate against glioma C6 depends on administration schedule and dosage. Exp Oncol. 2016 June; 38 (2): 80-3.
Fernandes J, et al., Perillyl alcohol induces apoptosis in human glioblastoma multiforme cells. Oncol Rep. 2005 May; 13 (5): 943-7.
Feucht D, et al., Preoperative growth dynamics of untreated glioblastoma: Description of an exponential growth type, correlating factors, and association with postoperative survival. Neurooncol Adv. 2024 Apr. 3; 6 (1): vdae053.
Karmur B, et al., Blood-brain barrier disruption in Neuro-Oncology: strategies, failures, and challenges to overcome. Front Oncol 10:563840, 2020.
Manduca N, et al., 3D cancer models: One step closer to in vitro human studies. Front Immunol. 2023 Apr. 11; 14:1175503.
Michelakis E, et al., Metabolic modulation of glioblastoma with dichloroacetate. Sci Transl Med. 2010 May 12; 2 (31): 31ra34.
Mohammed S, et al., Survival and quality of life analysis in glioblastoma multiforme with adjuvant chemoradiotherapy: a retrospective study. Rep Pract Oncol Radiother. 2022 Dec. 29; 27 (6): 1026-1036.
Morales D, et al., Intranasal delivery in glioblastoma treatment: prospective molecular treatment modalities. Heliyon 8: e09517, 2022.
Oraiopoulou M, et al., The temozolomide-doxorubicin paradox in glioblastoma in vitro-in silico preclinical drug-screening. Sci Rep. 2024 Feb. 14; 14 (1): 3759.
Rezk R, et al., Spatial heterogeneity of cell-matrix adhesive forces predicts human glioblastoma migration. Neurooncol Adv. 2020 Jul. 3; 2 (1): vdaa081.
Sharma P, et al., Tumor microenvironment in glioblastoma: Current and emerging concepts. Neurooncol Adv. 2023 Feb. 23; 5 (1): vdad009.
Shi L, et al., The DRD2 antagonist haloperidol mediates autophagy-induced ferroptosis to increase temozolomide sensitivity by promoting endoplasmic reticulum stress in glioblastoma. Clin Cancer Res. 2023 Aug. 15; 29 (16): 3172-3188.
Stensjøen A et al., Growth dynamics of untreated glioblastomas in vivo. Neuro Oncol. 2015 October; 17 (10): 1402-11.
Tataranni T, et al., Dichloroacetate (DCA) and cancer: an overview towards clinical applications. Oxid Med Cell Longev. 2019 Nov. 14; 2019:8201079.
Vollmann-Zwerenz A, et al., Tumor cell invasion in glioblastoma. Int J Mol Sci. 2020 Mar. 12; 21 (6): 1932.
Zhong J, et al., Mesenchymal migration as a therapeutic target in glioblastoma. J Oncol. 2010; 2010:430142.

1. A pharmaceutical composition, comprising:
(a) dichloroacetate,
(b) perillyl alcohol, and
(c) at least one pharmaceutically acceptable carriers or excipients.

2. The composition of claim 1, wherein the perillyl alcohol is S-(−)-perillyl alcohol.

3. The composition of claim 1, further comprising at least one further active agent.

4. The composition of claim 3, wherein the at least one further active agent is one or more of temozolomide, lomustine, bevacizumab, and a benzimidazole compound, and
wherein the benzimidazole compound is selected from one or more of albendazole, cambendazole, fenbendazole, flubendazole, mebendazole, oxibendazole, oxfendazole, thiabendazole, and triclabendazole.

5. The composition of claim 4, wherein the benzimidazole compound is flubendazole, mebendazole, or a combination thereof.

6. The composition of claim 3, wherein the at least one further active agent comprises at least octanoic acid.

7. The composition of claim 1, wherein:
(i) the composition is formulated for oral administration,
(ii) the composition is formulated for intranasal administration, or
(iii) the perillyl alcohol is formulated for intranasal administration and the dichloroacetate is formulated for oral administration.

8. A method of inhibiting the progression of, or treating, a brain tumor, or ameliorating at least one symptom associated with a brain tumor, in a subject in need thereof, which comprises administering a therapeutically effective amount of the pharmaceutical composition according to claim 1,
wherein the pharmaceutical composition inhibits the progression of, or treats, the brain tumor, or ameliorates at least one symptom associated with the brain tumor in the subject.

9. The method of claim 8, wherein the subject is human.

10. The method of claim 8, wherein the dichloroacetate and the perillyl alcohol are co-administered intranasally.

11. The method of claim 10, wherein the dichloroacetate and the perillyl alcohol are co-administered in a single dose.

12. The method of claim 8, wherein the dichloroacetate and the perillyl alcohol are co-administered at least once per day.

13. The method of claim 8, wherein the brain tumor is one or more of astrocytoma, brain stem glioma, chordoma, cranial base tumor craniopharyngioma, diffuse intrinsic pontine glioma, ependymoma, glioblastoma, glioblastoma multiforme, glioma, haemangioblastoma, lymphoma, medulloblastoma, meningioma, neuronal and glioneuronal tumors, oligodendroglioma, pineal tumors, pituitary adenoma, rhabdoid tumors vestibular secondary brain tumors due to metastasis, and schwannoma.

14. The method of claim 8, wherein the brain tumor is a glioma or a glioblastoma.

15. The method of claim 14, wherein the glioblastoma is a glioblastoma with a wild type isocitrate dehydrogenase 1 enzyme gene (IDH-wild-type) and/or with a wild type O⁶-methylguanine-DNA methyltransferase (MGMT) promoter.

16. The method of claim 8, wherein the brain tumor is resistant to perillyl alcohol treatment alone.

17. The method of claim 8, wherein the brain tumor is resistant to temozolomide.

18. The method of claim 8, further comprising administering at least one additional active agent to the subject.

19. The method of claim 18, wherein the additional active agent is one or more of temozolomide, lomustine, bevacizumab, and a benzimidazole compound, and
wherein the benzimidazole compound is one or more of albendazole, cambendazole, fenbendazole, flubendazole, mebendazole, oxibendazole, oxfendazole, thiabendazole, and triclabendazole.

20. The method of claim 19, wherein the benzimidazole compound is flubendazole, mebendazole, or a combination thereof.

21. The method of claim 18, wherein the at least one additional active agent comprises at least octanoic acid.

22. A kit, comprising:
one or more containers,
wherein the one or more containers comprise a therapeutically effective amount of the pharmaceutical composition according to claim 1.

23. The kit of claim of claim 22, wherein the perillyl alcohol is S-(−)-perillyl alcohol.

24. The kit of claim 22, further comprising at least one additional active agent.

25. The kit of claim 24, wherein the additional active agent is one or more of temozolomide, lomustine, bevacizumab, and a benzimidazole compound, and
wherein the benzimidazole compound is one or more of albendazole, cambendazole, fenbendazole, flubendazole, mebendazole, oxibendazole, oxfendazole, thiabendazole, and triclabendazole.

26. The kit of claim 25, wherein the benzimidazole compound is flubendazole, mebendazole, or a combination thereof.

27. A nasal spray comprising dichloroacetate and perillyl alcohol for the treatment of glioblastoma or glioma.
